(19) United States
(12) Patent Application Publication
(10) Pub. No.: US 2026/0009083 A1
(43) Pub. Date: Jan. 8, 2026

(54) PREDICTIVE MIRNAS FOR RESPONSE TO CANCER THERAPY

(71) Applicant: HUMMINGBIRD DIAGNOSTICS GMBH, HEIDELBERG (DE)

(72) Inventors: Timothy Rajakumar, Heidelberg (DE); Bruno Steinkraus, Heidelberg (DE); Julia Jehn, Heidelberg (DE); Rastislav Horos, Heidelberg (DE); Tobias Sikosek, Heidelberg (DE)

(21) Appl. No.: 18/559,300

(22) PCT Filed: Apr. 13, 2022
(86) PCT No.: PCT/EP2022/059970

§ 371 Date: Nov. 6, 2023

(30) Foreign Application Priority Data
May 20, 2021 (EP) 21174970.0
Sep. 13, 2021 (EP) 21196432.5

(51) Int. Cl. C12Q 1/6886; A61K 45/06

(52) U.S. Cl. CPC C12Q 1/6886; A61K 45/06; C12Q 2600/106; C12Q 2600/118; C12Q 2600/158; C12Q 2600/178

(57) ABSTRACT

The present invention relates to a method of predicting a response of a patient suffering from cancer. Further, the prevent invention relates to a method of providing a survival prognosis to a patient suffering from cancer. Furthermore, the present invention relates to a method of determining whether to treat a patient suffering from cancer. In addition, the present invention relates to a method of selecting a patient suffering from cancer to benefit from immunochemotherapy or immunotherapy. Moreover, the present invention relates to a kit for carrying out these methods.

### CROSS REFERENCE TO RELATED APPLICATIONS

This application is a U.S. National Stage of PCT/EP2022/059970, International Filing Date Apr. 13, 2022, which claims priority to EP 21174970.0, filed May 20, 2021, and EP 21196432.5, filed Sep. 13, 2021, the disclosures of all of the above are incorporated herein by reference for all purposes.

### REFERENCE TO SUBMISSION OF A SEQUENCE LISTING AS A TEXT FILE

The Sequence Listing written in file Sequence-Listing.txt created on Oct. 30, 2023, 12,288 bytes, machine format IBM-PC, MS-Windows operating system, is hereby incorporated by reference in its entirety for all purposes.

Please insert the SEQUENCE LISTING containing SEQ ID NOs:1-44 at the end of the application.

The present invention relates to a method of predicting a response to cancer therapy of a patient suffering from cancer. Further, the present invention relates to a method of providing a survival prognosis to a patient suffering from cancer. Furthermore, the present invention relates to a method of determining whether to treat a patient suffering from cancer. In addition, the present invention relates to a method of selecting a patient suffering from cancer to benefit from immunochemotherapy or immunotherapy. Moreover, the present invention relates to a kit for carrying out these methods.

### BACKGROUND OF THE INVENTION

Cancer is the uncontrolled growth of abnormal cells anywhere in a body. The abnormal cells are termed cancer cells, malignant cells, or tumor cells. Cancer cells can proliferate uncontrollably and form a mass of cancer cells.

Cancer therapy includes, but is not limited to, immunotherapy, chemotherapy, radiation therapy, vaccination therapy, stem cell therapy, anti-hormonal therapy, immunosuppressive therapy, antibody therapy, and surgical therapy. Standard clinical parameters such as tumor size, grade, lymph node involvement and tumor-node-metastasis (TNM) staging may correlate with outcome and severity to stratify patients with respect to cancer therapy regimens. However, stage-matched tumors grouped by histological subtypes may respond differently to the same treatment regimen.

Immunotherapy has recently gained recognition as highly effective therapy in late-stage cancers. Drugs used in immunotherapy work by blocking the mechanisms by which tumors commonly evade detection and unleashing the immune system to fight the cancer. Alongside the remarkable benefits, immunotherapies can be associated with characteristic (and sometimes severe) side effects. For this reason, it is required to administer immunotherapies only to those patients in which the benefits are predicted to outweigh the risks. The mainstay of response prediction to immunotherapies in the PD-1 inhibitor class (Nivolumab and Pembrolizumab) is the quantification of tumor PD-L1 expression. However, still only approximately 30% of patients will achieve a positive response.

In addition, in late-stage cancers, immunotherapy (IO) alone or immunotherapy in combination with chemotherapy (i.e. immunochemotherapy (ICT)) are recommended as treatment options in major international guidelines. However, there remains uncertainty as to the ideal therapeutic choice in individual patients.

A positive response to immunotherapy is dependent both on local interactions between malignant and immune cells in the tumour microenvironment as well as more distal interactions between the tumour and the peripheral immune system. The latter has the potential to provide non-invasive therapy guidance, but no blood-based biomarkers have yet entered routine clinical use.

There is, thus, a pressing need for more accurate biomarkers for cancer therapy, in particular immunotherapy, response prediction, and survival prediction. In addition, there is a pressing need for new biomarkers which can identify which patients will benefit from chemotherapy in addition to immunotherapy.

Multiple lines of evidence suggest that information held in peripheral blood may be predictive of the immune response within the localized tumor microenvironment. Associations have been found between immunotherapy response and peripheral blood counts and peripherally expanded T-cell clones.

The present inventors identified response and survival predictive miRNAs from whole blood for cancer therapy, in particular immunotherapy. These new miRNAs allow a quick and accurate clinical response prediction and survival prognosis in cancer diseases. Said miRNAs can thus be used as companion or complementary diagnostics in cancer therapy, in particular immunotherapy. As an additional advantage, repeated liquid biopsies reduce procedural risks and are more feasible than serial tumor biopsies (as required by the methods of the prior art) as a means to perform longitudinal monitoring and investigate the dynamic processes, e.g. of tumor PD-L1 expression. In addition, these new miRNAs allow to select a patient suffering from cancer to benefit from immunochemotherapy or immunotherapy.

### SUMMARY OF THE INVENTION

In a first aspect, the present invention relates to a method of predicting a response to cancer therapy of a patient suffering from cancer comprising the step of:
determining the level of at least one miRNA in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a second aspect, the present invention relates to a method of providing a survival prognosis to a patient suffering from cancer comprising the step of:
determining the level of at least one miRNA in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a third aspect, the present invention relates to a method of predicting side effects in cancer therapy of a patient comprising the step of:
determining the level of at least one miRNA in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a fourth aspect, the present invention relates to a method of monitoring a patient suffering from cancer comprising the step of:
determining the level of at least one miRNA in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a fifth aspect, the present invention relates to a method of determining whether to treat a patient suffering from cancer comprising the steps of
(i) carrying out the method according to the first aspect, thereby identifying the patient as patient who will respond to cancer therapy, carrying out the method according to the second aspect, thereby identifying the patient as patient having a good survival prognosis, or carrying out the method according to the third aspect, thereby identifying the patient as patient who will not suffer from side effects in cancer therapy, and
(ii) assigning the patient to (said) cancer therapy.

In a sixth aspect, the present invention relates to the use of at least one miRNA for predicting a response to cancer therapy of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a seventh aspect, the present invention relates to the use of at least one miRNA for providing a survival prognosis to a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In an eight aspect, the present invention relates to the use of at least one miRNA for predicting side effects in cancer therapy of a patient, wherein the at least one miRNA has a nucleotide sequence selected from the group consisting of SEQ ID NO: 1 to SEQ ID NO: 44, a fragment thereof, and a sequence having at least 80% sequence identity thereto.

In a ninth aspect, the present invention relates to the use of at least one miRNA for monitoring a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In a tenth aspect, the present invention relates to the use of at least one miRNA for determining whether to treat a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

In an eleventh aspect, the present invention relates to a kit for predicting a response to cancer therapy of a patient suffering from cancer, for providing a survival prognosis to a patient suffering from cancer, for predicting side effects in cancer therapy of a patient, for monitoring a patient suffering from cancer, or for determining whether to treat a patient suffering from cancer, wherein said kit comprises:
means for determining the expression level of at least one miRNA having a nucleotide sequence selected from the group consisting of SEQ ID NO: 1 to SEQ ID NO: 44, a fragment thereof, and a sequence having at least 80% sequence identity thereto.

In a twelfth aspect, the present invention relates to a method of selecting a patient suffering from cancer to benefit from immunochemotherapy or immunotherapy comprising the step of:
determining the level of at least one miRNA in a blood sample of a patient suffering from cancer, wherein the at least one miRNA has a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto, and
wherein the patient has optionally a PD-L1 Tumor Proportion Score (TPS) of ≥50%.

In a thirteenth aspect, the present invention relates to a method of determining whether to treat a patient suffering from cancer with immunochemotherapy or immunotherapy comprising the steps of
(i) carrying out the method according to the twelfth aspect, thereby identifying the patient as patient who will benefit from immunochemotherapy or immunotherapy, and
(ii) assigning the patient to said therapy.

In a fourteenth aspect, the present invention relates to a kit for selecting a patient suffering from cancer to benefit from immunochemotherapy or immunotherapy, wherein said kit comprises: means for determining the level of at least one miRNA having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto in a blood sample of a patient suffering from cancer, wherein the patient has optionally a PD-L1 Tumor Proportion Score (TPS) of ≥50%.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

As used herein, the term “about” indicates a certain variation from the quantitative value it precedes. In particular, the term “about” allows a ±5% variation from the quantitative value it precedes, unless otherwise indicated or inferred. The use of the term “about” also includes the specific quantitative value itself, unless explicitly stated otherwise. For example, the expression “about 80° C.” allows a variation of 4° C., thus referring to range from 76° C. to 84° C.

The term “biomarker”, as used herein, refers to a biological molecule found in blood, other body fluids, or tissues that is an indicator of a normal or abnormal process, or of a condition or disease. A biomarker may be used to foresee how well the body responds to a treatment for a disease or condition, or may be used to associate a certain disease or condition—or outcome of disease—to a certain value of said biomarker found in e.g. a blood sample. Biomarkers are also called molecular markers and signature molecules. If the biomarker is used to predict the probable course and outcome of a disease, it may be called a prognostic biomarker. The biomarkers described herein are miRNAs. Said miRNAs are predictive and prognostic biomarkers.

The term “miRNA” (the designation “microRNA” is also possible), as used herein, refers to a single-stranded RNA molecule of at least 10 nucleotides and of not more than 45 nucleotides covalently linked together. Preferably, the polynucleotides used in the present invention are molecules of 10 to 45 nucleotides or 15 to 35 nucleotides in length, more preferably of 16 to 28 nucleotides or 18 to 23 nucleotides in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length, not including optionally labels and/or elongated sequences (e.g. biotin stretches).

The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNA is initially transcribed as a longer precursor molecule (>1000 nucleotides long) called a primary miRNA transcript (pri-miRNA). Pri-miRNAs have hairpin structures that are processed by the Drosha enzyme (as part of the microprocessor complex). After Drosha processing, the pri-miRNAs are only 60-100 nucleotides long, and are called precursor miRNAs (pre-miRNAs). At this point, the pre-miRNA is exported to the cytoplasm, where it encounters the Dicer enzyme. Dicer cuts the miRNA in two, resulting in duplexed miRNA strands. Traditionally, only one of these miRNA arms was considered important in gene regulation: the arm that is destined to be loaded into the RNA-induced silencing complex (RISC), and occurs at a higher concentration in the cell. This is often called the “guide” strand and is designated as miR. The other arm is called the “minor miRNA” or “passenger miRNA”, and is often designated as miR*. It was thought that passenger miRNAs were completely degraded, but deep sequencing studies have found that some minor miRNAs persist and in fact have a functional role in gene regulation. Due to these developments, the naming convention has shifted. Instead of the miR/miR* name scheme, a miR-5p/miR-3p nomenclature has been adopted. By the new system, the 5′ arm of the miRNA is always designated miR-5p and the 3′ arm is miR-3p. The present nomenclature is as follows: The prefix “miR” is followed by a dash and a number, the latter often indicating order of naming. For example, hsa-miR-16 was named and likely discovered prior to hsa-miR-342. A capitalized “miR-” refers to the mature forms of the miRNA (e.g. hsa-miR-16-5p and hsa-miR-16-3p), while the uncapitalized “mir-” refers to the pre-miRNA and the pri-miRNA (e.g. hsa-mir-16), and “MIR” refers to the gene that encodes them. However, as this is a recent change, literature will often refer to the original miR/miR* names. After processing, the duplexed miRNA strands are loaded onto an Argonaute (AGO) protein to form a precursor to the RISC. The complex causes the duplex to unwind, and the passenger RNA strand is discarded, leaving behind a mature RISC carrying the mature, single stranded miRNA. The miRNA remains part of the RISC as it silences the expression of its target genes. While this is the canonical pathway for miRNA biogenesis, a variety of others have been discovered. These include Drosha-independent pathways (such as the mirtron pathway, snoRNA-derived pathway, and shRNA-derived pathway) and Dicer-independent pathways (such as one that relies on AGO for cleavage, and another which is dependent on tRNaseZ).

The term “miRBase”, as used herein, refers to a well-established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download.

The miRNAs described herein have a nucleotide sequence selected from the group consisting of SEQ ID NO: 1 to SEQ ID NO: 44, a fragment thereof, and a sequence having at least 80%, preferably at least 85%, more preferably at least 90%, even more preferably at least 95%, or still even more preferably at least 99%, e.g. at least 80, 81, 82, 83, 84, 85, 86, 87, 88, 89, 90, 91, 92, 93, 94, 95, 96, 97, 98, or 99%, sequence identity thereto.

Residues in two or more polynucleotides are said to “correspond” to each other if the residues occupy an analogous position in the polynucleotide structures. It is well known in the art that analogous positions in two or more polynucleotides can be determined by aligning the polynucleotide sequences based on nucleic acid sequence or structural similarities. Such alignment tools are well known to the person skilled in the art and can be, for example, obtained on the World Wide Web, for example, ClustalW or Align using standard settings, preferably for Align EMBOSS::needle, Matrix: Blosum62, Gap Open 10.0, Gap Extend 0.5.

The term “disease”, as used herein, refers to an abnormal condition that affects the body of an individual. A disease is often construed as a medical condition associated with specific symptoms and signs. In humans, the term “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “cancer”, as used herein, refers to or describes a physiological condition in an individual that is typically characterized by unregulated cell growth. Examples of cancer include, but are not limited to, lung cancer, preferably non-small-cell lung carcinoma (NSCLC) or small-cell lung carcinoma (SCLS), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia. The term “cancer”, as used herein, also encompasses cancer metastases.

The term “lung cancer”, as used herein, refers to a disease which consists of uncontrolled cell growth in tissues of the lung. This growth may lead to metastasis, which is the invasion of adjacent tissue and infiltration beyond the lungs. The vast majority of primary lung cancers are carcinomas of the lung, derived from epithelial cells. Lung cancer is the most common cause of cancer-related death in men and women. The most common symptoms are shortness of breath, coughing (including coughing up blood), and weight loss. The lung cancer may be late-stage lung cancer, in particular lung cancer of stage IV. The main types of lung cancer are non-small cell lung carcinoma (NSCLC) and small cell lung carcinoma (SCLC). Preferably, the lung cancer is NSCLC, specifically of stage IV.

The term “tumor”, as used herein, refers to a lesion and neoplastic cell growth and proliferation, whether malignant or benign, and all pre-cancerous and cancerous cells and tissues resulting in abnormal tissue growth.

The term “therapeutic treatment/therapy”, as used herein, relates to any treatment/therapy which improves the health status and/or prolongs (increases) the lifespan of a patient. Said treatment/therapy may eliminate the disease in a patient, arrest, inhibit, or slow the development of a disease in a patient, decrease the frequency or severity of symptoms in a patient, and/or decrease the recurrence in a patient who currently has or who previously has had a disease.

As used herein, the term “cancer therapy” refers to any protocol, method, and/or agent that can be used in the prevention, management, treatment, and/or amelioration of a cancer. In particular, the term “cancer therapy”, as used herein, means accomplishing one or more of the following: (i) tumor growth inhibition and/or tumor cell death, (ii) reduction of tumor marker(s), (iii) reduction of tumor lesions and metastases, (iv) reduction of tumor burden as evidenced by imaging studies (e.g. computer tomography (CT), magnetic resonance imaging (MRI), positron emission tomography (PET), etc.), and (v) reduction of tumor burden as evidenced by clinical appraisal or self-report by the patient. Specifically, cancer therapy includes, but is not limited to, drug therapy, supportive therapy, and/or other therapy useful in the prevention, management, treatment, and/or amelioration of cancer known to one of skill in the art, such as medical personnel. More specifically, cancer therapy includes, but is not limited to, immunotherapy, chemotherapy, radiation therapy, vaccination therapy, stem cell therapy, anti-hormonal therapy, immunosuppressive therapy, antibody therapy, and/or surgical therapy. Even more specifically, the cancer therapy is immunotherapy.

The term “chemotherapy”, as used herein, relates to a type of cancer treatment that uses one or more anti-cancer drugs, also known as chemotherapeutic agents, as part of a standardized chemotherapy regimen. Chemotherapy may be given with a curative intent (which preferably involves combinations of drugs), or it may aim to prolong life or to reduce symptoms (palliative chemotherapy). Preferably, the chemotherapy is platinum doublet chemotherapy.

The term “chemotherapeutic agent”, as used herein, refers to any agent which directly or indirectly inhibits the uncontrolled growth and proliferation of cancer cells. The chemotherapeutic agent includes, but is not limited to, an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and/or an inhibitor of histone deacetylase. Preferably, the chemotherapeutic agent is a platinum doublet.

The term “radiation therapy”, as used herein, relates to a type of cancer treatment using ionizing radiation to control or kill cancerous/malignant cells. Ionizing radiation is normally delivered by a linear accelerator. Radiation therapy may be curative in several types of cancer if they are localized to one area of the body. It may also be used as part of adjuvant therapy, to prevent tumor recurrence after surgery to remove a primary malignant tumor. Radiation therapy is synergistic with chemotherapy, and can used before, during, and after chemotherapy in susceptible cancers.

The term “immunotherapy” refers to any therapy in which one or more components of a human's or animal's immune system is (are) deliberately modulated in order to directly or indirectly achieve some therapeutic benefit, including systemic and/or local effects, and preventative and/or curative effects. Immunotherapy can involve administering one or more immunotherapeutic drugs or agents, either alone or in any combination, to a human or animal subject by any route (e.g. orally, intravenously, dermally, by injection, by inhalation, etc.), whether systemically, locally or both. Immunotherapy can involve provoking, increasing, decreasing, halting, preventing, blocking or otherwise modulating the production of cytokines, and/or activating or deactivating cytokines or immune cells, and/or modulating the levels of immune cells, and/or delivering one or more therapeutic or diagnostic substances to a particular location in the body or to a particular type of cell or tissue, and/or destroying particular cells or tissue. Immunotherapy can be used to achieve local effects, systemic effects, or a combination of both. Immunotherapy can also be used to reduce or eliminate side effects that may have been caused by other anti-cancer therapies.

Immunotherapies designed to elicit or amplify an immune response are classified as activation immunotherapies, while immunotherapies that reduce or suppress an immune response are classified as suppression immunotherapies.

An antitumor immunotherapy has broad potential and can be used to treat many different types of advanced-stage cancer owing to the durable and robust responses it elicits across a diverse spectrum of malignancies.

Immunotherapy encompasses the administration of an immunotherapeutic agent.

The term “immunotherapeutic agent”, as used herein, refers to any drug, compound, or biologic that indirectly or directly restores, enhances, stimulates, or increases the body's immune response against cancer cells and/or that decreases the side effects of other anticancer therapies. Examples of common immunotherapeutic agents known in the art include, but are not limited to, checkpoint inhibitors, cytokines, cancer vaccines, antibodies such as monoclonal antibodies, and/or non-cytokine adjuvants. Alternatively, or additionally, the immunotherapeutic treatment comprises the administration of immune cells (e.g. T cells, NK, cells, dendritic cells, B cells, etc.) to the patient. Especially, said immune cells are antigen-presenting cells and/or chimeric antigen receptor T cells.

Immunotherapeutic agents can be non-specific, i.e. boost the immune system generally so that the human body becomes more effective in fighting the growth and/or spread of cancer cells, or they can be specific, i.e. targeted to the cancer cells themselves. Immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins, and/or colony-stimulating factors. Interferons (IFNs) include, for example, IFN-alpha (IFN-α), IFN-beta (IFN-β), and/or IFN-gamma (IFN-γ). Interleukins include, for example, IL-2, IL-4, IL-11, and/or IL-12. Colony-stimulating factors (CSFs) include, for example, granulocyte colony stimulating factor (G-CSF or filgrastim), granulocyte-macrophage colony stimulating factor (GM-CSF or sargramostim), and/or erythropoietin (epoetin alfa, darbopoietin).

In addition, immunotherapeutic agents can be active, i.e. stimulate the body's own immune response including humoral and cellular immune responses, or they can be passive, i.e. comprise immune system components such as antibodies, effector immune cells, antigen-presenting cells etc. that were generated external to the body. In particular, passive immunotherapy involves the use of one or more monoclonal antibodies that are specific for a particular antigen found on the surface of a cancer cell or immune cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

It is known that tumors evade immune-mediated recognition through multiple mechanisms of immune escape. On chronic tumor antigen exposure, T cells become dysfunctional/exhausted and upregulate various checkpoint inhibitory receptors (IRs) that limit T cells' survival and function. Thus, immunotherapy particularly encompasses an approach of treating cancer by stimulating T-cell function and preventing T-cell death. This approach is based on the striking finding that stimulation of T-cell function with compounds that block or activate regulatory receptors, e.g. antibodies, can be sufficient to cause the regression of tumors. Checkpoint blockade is, for example, a method by which T-cell function is stimulated with compounds that block their inhibitory receptors, whereas T-cell co-stimulation is a method that aims at activating T-cell function with compounds that target their stimulatory receptors. The immunotherapy described herein specifically encompasses the administration of a checkpoint inhibitor.

In one embodiment of the present invention, the immunotherapeutic agent is a PD-1/PD-L1 inhibitor. In one preferred embodiment of the present invention, the PD-1/PD-L1 inhibitor is selected from the group consisting of pembrolizumab, nivolumab, cemiplima, durvalumab, and atezolizumab. In this respect, it should be noted that pembrolizumab and nivolumab specifically target the PD-1 protein, while atezolizumab, durvalumab, and avelumab specifically target the PD-L1 ligand. PD-1 means programmed cell-death protein 1 and PD-L1 means programmed cell-death ligand 1. PD-1 is the receptor for the ligand PD-L1.

As used herein, the term “immune checkpoint inhibitor” refers to any compound that totally or partially reduces, inhibits, interferes with or modulates one or more immune checkpoint proteins. Inhibition includes reduction of function and full blockade. A number of immune checkpoint inhibitors are known and in analogy of these known immune checkpoint protein inhibitors, alternative immune checkpoint inhibitors may be developed in the (near) future. The immune checkpoint inhibitor includes, but is not limited to, a peptide, an antibody, a nucleic acid molecule, and/or a small molecule. Preferably, the immune checkpoint inhibitor is selected from the group consisting of an inhibitor targeting programmed cell-death protein 1 (PD-1), programmed cell-death ligand 1 (PD-L1), programmed cell-death ligand 2 (PD-L2), cytotoxic T-lymphocyte-associated Protein 4 (CTLA-4), LAG3 (Lymphocyte Activation Gene-3), TIGIT (T cell immunoreceptor with immunoglobulin and ITIM domain), CD73 (an ecto-5′-nucleotidase (NT5E)), and intrinsic checkpoint blockades. More preferably, the inhibitor targeting PD-L1 is selected from the group consisting of atezolizumab, durvalumab, and avelumab and the inhibitor targeting PD-1 is selected from the group consisting of pembrolizumab and nivolumab.

The term “prediction”, as used herein, refers to a prognosis whether a patient will respond to a cancer therapy or achieve a clinical outcome in response to a cancer therapy. In some embodiments, a positive response to a cancer therapy is predicted. In some alternative embodiments, a negative response to cancer therapy is predicted. The prediction of a response of a patient to a cancer therapy is usually carried out before treating the patient with the cancer therapy. If the predicted outcome is positive, the patient will receive the cancer therapy. If the predicted outcome is negative, the patient will receive no cancer therapy or an alternative cancer therapy. For example, if it is predicted that the patient suffering from a cancer will not respond to an immunotherapy, the patient may be treated with another therapy than immunotherapy such as radiotherapy and/or chemotherapy. However, the prediction of a response to a cancer therapy may also be carried out after the start of a cancer therapy. If the predicted outcome is positive, the cancer therapy is continued. If the predicted outcome is negative, the cancer therapy is changed or stopped.

In particular, the method of predicting a response to cancer therapy of a patient suffering from cancer, as described herein, is suitable for discriminating patients who will respond to cancer therapy from patients who will not respond to cancer therapy. Typically, the characterization of a patient as responder or non-responder can be performed by reference to a standard or training set. The standard may be the profile of subjects who are known to be responders or non-responders or alternatively may be a numerical value/reference level. Such predetermined standards may be provided in any suitable form, such as a printed list or diagram, computer software program, mathematical algorithm, or other media. When the patient is predicted as being a responder, the physician can take the decision to administer the cancer therapy, e.g. immunotherapy, to the patient. When the patient is predicted as being a non-responder, the physician can take the decision not to administer the cancer therapy but instead administer an alternative cancer therapy. In this case, any further adverse sides effects may be avoided.

The present inventors have identified new miRNA biomarkers allowing the prediction of a response to cancer therapy of a patient suffering from cancer. Said miRNAs have a nucleotide sequence selected from the group consisting of SEQ ID NO: 1 to SEQ ID NO: 44, a fragment thereof, and a sequence having at least 80% sequence identity thereto. The cancer therapy is preferably an immune therapy.

The term “response”, as used herein, refers to an alteration in a patient's condition that occurs as a result of or correlates with cancer treatment. In some embodiments, a response is or comprises a beneficial response. In some embodiments, a beneficial response may include stabilization of the condition (e.g. prevention or delay of deterioration expected or typically observed to occur in the absence of treatment), amelioration (e.g. reduction in frequency and/or intensity) of one or more symptoms of the condition, and/or improvement in the prospects for cure of the condition, etc. Particularly, the response is the response of the patient suffering from cancer to immunotherapy.

Specifically, a cancer patient or (control) subject suffering from cancer who has been treated with a cancer therapy is considered to “respond”, have a “response”, have “a positive response” or be “responsive” to the cancer therapy, if the individual shows evidence of an anti-cancer effect according to an art-accepted set of objective criteria or reasonable modification thereof, including a clinically significant benefit, such as the prevention, or reduction of severity, of symptoms, or a slowing of the progression of the cancer. It will be understood that the aforementioned terms may also be used in regard to the cancer.

A variety of different objective criteria for assessing the effect of anti-cancer treatments on cancers are known in the art. The World Health Organization (WHO) criteria (Miller, A B, et al., Cancer 1981; 47(1):207-14) and modified versions thereof, the Response Evaluation Criteria in Solid Tumors (RECIST) (Therasse P, et al., J Natl Cancer Inst 2000; 92:205-16), and revised version thereof (Eisenhauer E A, New response evaluation criteria in solid tumors: revised RECIST guideline (version 1.1). Eur J Cancer 2009; 45(2):228-47) are sets of objective criteria, based on imaging measurements of the size and number of tumor lesions and detection of new lesions, e.g. from computed tomography (CT), magnetic resonance imaging (MRI), or conventional radiographs. Dimensions of selected lesions (referred to as target lesions) are used to calculate the change in tumor burden between images from different time points. The calculated response is then categorized as complete response (CR), partial response (PR), stable disease (SD), or progressive disease (PD). CR is complete disappearance of tumor (−100%), and PD is an increase of about 20%-25% or greater (depending on the particular criteria) and/or the appearance of new lesions. PR is a significant reduction (of at least about 30%) in size of tumor lesions (without emergence of new lesions) but less than a complete response. SD is in between PR and PD.

However, in some cases, anatomic imaging alone may be not sufficient. For example, meaningful tumor responses to immunotherapy, e.g. with immune checkpoint inhibitors, may occur after a delay, in some cases following the above described WHO or RECIST criteria. Immune-related response criteria (irRC) were defined in an attempt to capture additional favorable response patterns observed with immune therapies (Wolchok, J D, et al. (2009) Guidelines for the evaluation of immune therapy activity in solid tumors: immune-related response criteria. Clin. Care Res. 15, 7412-7420.). The developers of the irRC based their criteria on the WHO Criteria but modified it: In the irRC, tumor burden is measured by combining ‘index’ lesions with new lesions. Ordinarily tumor burden would be measured simply with a limited number of ‘index’ lesions (that is, the largest identifiable lesions) at baseline, with new lesions identified at subsequent timepoints counting as ‘Progressive Disease’. In the irRC, by contrast, new lesions are simply a change in tumor burden. The irRC retained the bidirectional measurement of lesions that had originally been laid down in the WHO Criteria. In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). The thinking here is that even if tumor burden is rising, the immune system is likely to ‘kick in’ some months after first dosing and lead to an eventual decline in tumor burden for many patients. The 25% threshold allows this apparent delay to be accounted for.

The irRC are applicable to immune checkpoint inhibitors and other immunotherapeutic agents. One of ordinary skill in the art will appreciate that additional response criteria are known in the art, which take into consideration various factors such as changes in the degree of tumor arterial enhancement and/or tumor density as indicators of tumor viable tissue, with decreased arterial enhancement and decreased tumor density being indicators of reduced viable tumor tissue (e.g. due to tumor necrosis).

By contrast, a cancer patient or (control) subject suffering from cancer who has been treated with a cancer therapy is considered “not to respond”, “to lack a response”, to have “a negative response” or be “non-responsive” to the cancer therapy, if the therapy provides no clinically significant benefit, such as the prevention, or reduction of severity, of symptoms, or increases the rate of progression of the cancer.

The term “prognosis”, as used herein, means the likelihood of recovery from a disease or the prediction of the probable development or outcome of a disease, including but not limited to, predicting the survival, in particular overall survival, of the patient, the likelihood of reappearance of cancer in a patient, and the likelihood of tumor metastasis.

The term “survival prognosis”, as used herein, refers to the prediction of the likelihood/probability of death of a patient suffering from cancer. In particular, the term “providing a survival prognosis to a patient suffering from cancer”, as used herein, means determining whether the patient has a good prognosis (low probability of death) or a poor prognosis (high probability of death) with respect to cancer. The patient is preferably a patient to whom a cancer therapy will be, is, or has been administered. The survival prognosis may be defined as the predicted overall survival (OS) and/or survival at 2- or 5-years follow-up. A survival chance/probability can be expressed in a value of between 0% and 100%, where 100% is a high chance/probability of surviving an indicated time period (good prognosis) and 0% is a low probability/chance of surviving an indicated time period (poor prognosis). The indicated time period may be (at least/at most) 3 months, 6 months, 9 months, 12 months (1 year), 2 years, 3 years, 4 years, 5 years, 6 years, 7 years, 8 years, 9 years, or 10 years.

Specifically, a good survival prognosis is associated with a high chance/probability of surviving a certain time period. More specifically, a patient having a good prognosis has a chance/probability of greater than 50%, preferably of greater than 60%, more preferably of greater than 70%, even more preferably of greater than 80%, still even more preferably of greater than 90%, or most preferably of greater than 95% to survive a certain time period. The certain time period is preferably a time period of (at least) 3 months, 6 months, 9 months, 12 months (1 year), 2 years, 3 years, 4 years, 5 years, 6 years, 7 years, 8 years, 9 years, or 10 years.

Specifically, a poor survival prognosis is associated with a low chance/probability of surviving a certain time period. More specifically, the patient having a low prognosis has a chance/probability of equal to or lower than 50%, preferably lower 40%, more preferably of lower than 30%, even more preferably of lower than 20%, still even more preferably of lower than 10%, and most preferably of lower than 5% to survive a certain time period. The certain time period is preferably a time period of (at most) 3 months, 6 months, 9 months, 12 months (1 year), 2 years, 3 years, 4 years, 5 years, 6 years, 7 years, 8 years, 9 years, or 10 years.

Alternatively, it is predicted that the patient suffering from cancer has a short or long survival time. The term “short survival time”, e.g. (at most) 3 months, 6 months, 9 months, or 12 months (1 year), indicates that the patient will have a survival time that will be lower than the median (or mean) observed in the general population of (control) subjects suffering from said cancer. When the patient will have a short survival time, it is meant that the patient has a “poor prognosis”. Inversely, the term “long survival time”, e.g. (at least) 2 years, at least 5 years, at least 8 years, or at least 10 years, indicates that the patient will have a survival time that will be higher than the median (or mean) observed in the general population of (control) subjects suffering from said cancer. When the patient will have a long survival time, it is meant that the patient has a “good prognosis”. For example, a patient who will have a predicted short survival time will not be alive at a 2- or 5-years follow-up anymore, while a patient who will have a predicted long survival time will still be alive at 2- or 5-years follow up.

Those of skill in the art will recognize that an “overall survival (OS)” time is generally based on and expressed as the percentage of people who survive a certain type of cancer for an indicated time period. Cancer statistics often use an overall 2-year or 5-year survival rate. In a specific embodiment, patients having a good prognosis will survive a time period of 2-years or 5-years. Patients having a poor prognosis will not survive a time period of 2-years or 5-years.

The method of providing a survival prognosis to a patient suffering from cancer is a valuable tool in predicting whether (overall or long-term) survival of the individual, e.g. following therapy, is likely. This method can also be used clinically to make treatment decisions by choosing the most appropriate treatment modalities for any particular patient suffering from cancer. The treatment modalities may be selected from the group consisting of immunotherapy, chemotherapy, radiation therapy, vaccination therapy, stem cell therapy, anti-hormonal therapy, immunosuppressive therapy, antibody therapy, and surgical therapy.

The Kaplan-Meier analysis is the recommended statistical technique for survival analysis. It is applied by analyzing the distribution of patient survival times following their recruitment to a study. The analysis expresses these in terms of the proportion of patients still alive up to a given time following recruitment. In graphical terms, a plot of the proportion of patients surviving against time has a characteristic decline (often exponential), the steepness of the curve indicating the efficacy of the treatment being investigated. The shallower the survival curve, the more effective the treatment. Kaplan-Meier analysis can be used to test the statistical significance of differences between the survival curves associated with two different treatments.

The present inventors have identified with this kind of analysis new miRNA biomarkers allowing the provision of a survival prognosis to a patient suffering from cancer. These miRNAs have a nucleotide sequence selected from the group consisting of SEQ ID NO: 1 to SEQ ID NO: 44, a fragment thereof, and a sequence having at least 80% sequence identity thereto. The present inventors have found that said miRNAs are discriminators of cancer patients who have been treated with a cancer therapy regarding their survival time. They are prognostic indicators allowing to stratify/classify high and low risk patients.

The term “PD-L1 Tumor Proportion Score (TPS)”, as used herein, refers to the percentage of PD-L1 positive tumor cells out of all vital tumor cells. Thus, in case of determining the PD-L1 Tumor Proportion Score (TPS), all tumor cells that stain membrane-bound positive for PD-L1 are counted and divided by the total number of tumor cells (percentage value of positive tumor cells). The PD-L1 TPS may be a decision criterion in the selection of a treatment regimen. Particularly, the treatment regimen of a patient suffering from cancer having a PD-L1 TPS of ≥50% may differ from the treatment regimen of a patient suffering from cancer having a PD-L1 TPS of <1%, and/or from the treatment regimen of a patient suffering from cancer having a PD-L1 TPS of between 1 to 49%.

In late stage lung cancer such as stage IV NSCLC, for example, patients having a PD-L1 TPS of <1% are usually treated with chemotherapy and patients having a PD-L1 TPS of between 1 to 49% are usually treated with immunochemotherapy.

Currently, both immunotherapy and immunochemotherapy are recommended as treatment options for patients with late stage lung cancer such as stage IV NSCLC and PD-L1 TPS≥50%. Some of the approximately 60% of patients who will fail to respond to immunotherapy may benefit from immunochemotherapy, as the chemotherapy component can sensitise the tumour to concurrent immunotherapy. This additional therapeutic burden, however, comes at the cost of increased frequency and severity of toxicity, with grade 3-4 adverse events noted in approximately 70% of patients. The choice of either immunotherapy and immunochemotherapy remains largely based on clinical judgement. Factors that may be considered include general health status, number of metastatic sites and disease aggressiveness. However, the optimal therapy is still often unclear. The present inventors have solved this problem. They have defined a (miRisk) cut-off score which allows to identify a patient who will benefit from treatment with immunochemotherapy as opposed to immunotherapy. In this way, a patient only has to undergo immunotherapy in combination with chemotherapy and the associated high physical stress if the patient really needs it. This (miRisk) cut-off score, thus, allows treatment decisions as a complementary diagnostic.

The term “patient”, as used herein, refers to any individual suffering from cancer for whom it is desired to know whether she or he will respond to cancer therapy. It may be predicted whether the patient suffering from cancer will respond to cancer therapy or will not respond to cancer therapy. If the patient is identified as patient who will respond to cancer therapy, the patient is assigned to said cancer therapy. If the patient is identified as patient who will not respond to cancer therapy, the patient is not assigned to said cancer therapy, but may be assigned to an alternative cancer therapy.

The term “patient”, as used herein, also refers to any individual suffering from cancer for whom a survival prognosis is desired to know. It may be determined that the patient suffering from cancer has a good or poor survival prognosis.

The term “patient”, as used herein, also refers to any individual suffering from cancer for whom it is desired to know how cancer develops in the patient. This patient may be monitored, e.g. in form of a longitudinal monitoring. Thus, the course of cancer (particularly after or during treatment) may be observed in the patient.

The term “patient”, as used herein, also refers to any individual suffering from cancer for whom it is desired to know whether the patient will (likely) benefit from immunotherapy or immunochemotherapy (i.e. patient selection/stratification).

The patient may be any mammal, including both a human and another mammal, e.g. an animal such as a rabbit, mouse, rat, or monkey. Human patients are particularly preferred.

The term “(control) subject”, as used herein, refers to an individual suffering from cancer who is a known responder or non-responder of cancer therapy. The term “(control) subject”, as used herein, also refers to an individual suffering from cancer having a known good or poor survival prognosis.

The (control) subject may be any mammal, including both a human and another mammal, e.g. an animal such as a rabbit, mouse, rat, or monkey. Human control subjects are particularly preferred.

The term “blood sample”, as used herein, encompasses whole blood or a blood fraction. Preferably, the blood fraction is selected from the group consisting of a blood cell fraction, plasma, and serum. In particular the blood fraction is selected from the group consisting of a blood cell fraction and plasma or serum. For example, the blood cell fraction encompasses erythrocytes, leukocytes, and/or thrombocytes. More preferably, the blood cell fraction is a fraction of leukocytes. Specifically, leukocytes encompass myeloid cells and/or lymphocytes. More specifically, the myeloid cells are selected from the group consisting of granulocytes, monocytes, macrophages, and dendritic cells (DCs), or are a combination thereof.

It is preferred that the blood sample has a volume of between 0.01 and 20 ml, more preferably of between 0.1 and 10 ml, even more preferably of between 0.5 and 8 ml and most preferably of between 1 and 5 ml.

Said blood sample may be provided by removing blood from a patient or (control) subject, but may also be provided by using a previously isolated sample. For example, a blood sample may be taken from a patient or (control) subject by conventional blood collection techniques.

The blood sample may further be obtained from a patient or (control) subject prior to the initiation of a therapeutic treatment, during the therapeutic treatment, and/or after the therapeutic treatment. If the blood sample is obtained from at least one (control) subject, e.g. from at least 1, 2, 3, 4, 5, 6, 7, 8, 9, 10, 20, 30, 40, 50, 60, 70, 80, 90, 100, 150, 200, 250, 300, 400, 500, or 1,000 (control) subject(s), it is designated as “reference blood sample”. Preferably, the reference blood sample is from the same source than the blood sample of the patient to be tested, e.g. both are whole blood samples or blood cell fractions. It is further preferred that both are from the same species, e.g. from a human. It is also (alternatively or additionally) preferred that the measurements of the reference blood sample of the (control) subject and the blood sample of the patient to be tested are identical, e.g. both have an identical volume. It is particularly preferred that the reference blood sample and the blood sample are from (control) subjects/patients of the same sex and similar age.

The whole blood sample may be collected by means of a blood collection tube. It is, for example, collected in a PAXgene Blood RNA tube, in a Tempus Blood RNA tube, in an EDTA-tube, in a Na-citrate tube, Heparin-tube, or in a ACD-tube (Acid citrate dextrose).

The whole blood sample may also be collected by means of a bloodspot technique, e.g. using a Mitra Microsampling Device. This technique requires smaller sample volumes, typically 45-60 μl for humans or less. For example, the whole blood may be extracted from the patient via a finger prick with a needle or lancet. Thus, the whole blood sample may have the form of a blood drop. Said blood drop is then placed on an absorbent probe, e.g. a hydrophilic polymeric material such as cellulose, which is capable of absorbing the whole blood. Once sampling is complete, the blood spot is dried in air before transferring or mailing to labs for processing. Because the blood is dried, it is not considered hazardous. Thus, no special precautions need be taken in handling or shipping. Once at the analysis site, the desired components, e.g. miRNAs, are extracted from the dried blood spots into a supernatant which is then further analyzed. This technique is suitable for monitoring patients having cancer at home (on a home care/home sampling basis) or for screening purposes.

The term “myeloid cells”, as used herein, refers to a subgroup of leukocytes. This subgroup includes granulocytes, monocytes, macrophages, and dendritic cells (DCs). In other words, granulocytes, monocytes, macrophages, and dendritic cells (DCs) are collectively called myeloid cells. In one embodiment, the myeloid cells are selected from the group consisting of granulocytes, monocytes, macrophages, and dendritic cells (DCs), or are a combination thereof. Myeloid cells circulate through the blood and lymphatic system and are rapidly recruited to sites of tissue damage, including tumor associated tissue damage, and infection via various chemokine receptors. Thus, myeloid cells represent a critical arm of the immune system, largely responsible for innate defence against ani array of pathogens. Myeloid cells also play a key role in linking innate and adaptive immunity, primarily through antigen presentation and recruitment of adaptive immune cells. As mentioned above, the present inventors identified new miRNA biomarkers for survival prognosis and response prediction. Some of them specifically/exclusively occur in myeloid cells. These miRNAs have a nucleotide sequence selected from the group consisting of SEQ ID NO: 1, SEQ ID NO: 5, SEQ ID NO: 7, SEQ ID NO: 8, SEQ ID NO: 12, SEQ ID NO: 17, SEQ ID NO: 19, SEQ ID NO: 28, SEQ ID NO: 31, SEQ ID NO: 34, SEQ ID NO: 38, SEQ ID NO: 41 to SEQ ID NO: 44, a fragment thereof, and a sequence having at least 80% sequence identity thereto. Said miRNAs are designed as myeloid miRNAs herein.

In the methods described herein, the level of a miRNA is determined in a blood sample of a patient. The term “level”, as used herein, refers to an amount (measured for example in grams, mole, or ion counts) or concentration (e.g. absolute or relative concentration, e.g. reads per million (RPM) or NGS counts) of the miRNA. The term “level”, as used herein, also comprises scaled, normalized, or scaled and normalized amounts or values (e.g. RPM). In particular, the level of the miRNA is determined by sequencing, preferably next generation sequencing (e.g. ABI SOLID, Illumina Genome Analyzer, Roche 454 GS FL, BGISEQ), nucleic acid hybridization (e.g. microarray or beads), nucleic acid amplification (e.g. PCR, RT-PCR, qRT-PCR, or high-throughput RT-PCR), polymerase extension, mass spectrometry, flow cytometry (e.g. LUMINEX), or any combination thereof. Specifically, the level of the miRNA is the expression level of said miRNA.

Those of skill in the art will appreciate that, in many embodiments described herein, the determined miRNA level is compared with an appropriate miRNA “reference level”. Specifically, the level of the miRNA is compared to a reference level of said miRNA. More specifically, the reference level of a miRNA is determined in a blood sample of (control) subjects. Even more specifically, the reference level is determined empirically by measuring a number of reference blood samples from subjects suffering from cancer known to be responders or non-responders to cancer therapy or by measuring a number of reference blood samples from subjects suffering from cancer having a known good or poor survival prognosis. Typically, as would be understood by those skilled in the art, the reference level is determined under conditions comparable to those utilized to determine or analyze the miRNA level in a blood sample of a patient.

The reference level may also be a cut-off or threshold level of the miRNA. Typically, a cut-off or threshold level can be determined experimentally, empirically, or theoretically. A cut-off or threshold level can also be arbitrarily selected based upon the existing experimental and/or clinical conditions, as would be recognized by a person of ordinary skilled in the art. The cut-off or threshold level must be determined in order to obtain the optimal sensitivity and specificity according to the function of the test and the benefit/risk balance (clinical consequences of false positive and false negative). Typically, the optimal sensitivity and specificity (and so the threshold level) can be determined using a Receiver Operating Characteristic (ROC) curve based on experimental data. For example, after determining the miRNA level in a group of a reference, one can use algorithmic analysis for the statistic treatment of the measured miRNA level in samples to be tested, and thus obtain a classification standard having significance for sample classification. The full name of ROC curve is receiver operator characteristic curve, which is also known as receiver operation characteristic curve. It is mainly used for clinical biomarker tests. The ROC curve is a comprehensive indicator that reflects the continuous variables of true positive rate (sensitivity) and false positive rate (1−specificity). It reveals the relationship between sensitivity and specificity with the image composition method. A series of different cut-off or threshold levels are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of a prediction/prognosis/diagnosis. On the ROC curve, the point closest to the far upper left of the coordinate diagram is a critical point having both high sensitivity and high specificity values. The AUC value of the ROC curve is between 1.0 and 0.5. When AUC>0.5, the predicted/prognosed/diagnosed result gets better and better as AUC approaches 1. When AUC is between 0.5 and 0.7, the accuracy is low. When AUC is between 0.7 and 0.8, the accuracy is moderate. When AUC is higher than 0.8, the accuracy is high. When AUC is higher than 0.9, the accuracy is very high.

The term “classifier”, as used herein, refers to a prediction/prognostic model which allows to distinguish between or characterize samples by classifying a given sample into a predetermined class based on certain characteristics of said sample. For example, a classification, as used herein, is capable of predicting with a relatively high sensitivity and specificity if a blood sample of a patient of unknown response prediction belongs to the class of one of two given classes; each class representing a predicted estimated response. The output may be given as a probability of belonging to either class of between 0-1. A classification, as used herein, is also capable of predicting with a relatively high sensitivity and specificity if a blood sample of a patient of unknown survival prognosis belongs to the class of one of two given classes; each class representing a predicted estimated survival. The output may be given as a probability of belonging to either class of between 0-1. Specifically, a classifier allows to distinguish responders to cancer therapy from non-responders to cancer therapy. A classifier may also allow to distinguish patients having a poor survival prognosis from patients having a good survival prognosis. In addition, a classifier may also allow to distinguish whether the patient should be assigned to and/or treated with immunotherapy or immunochemotherapy.

The term “pharmaceutically effective amount”, as used herein, refers to an amount which achieves a desired reaction or a desired effect alone or together with further doses. In case of the treatment of a particular disease, the desired reaction preferably relates to an inhibition of the course of the disease. This comprises slowing down the progress of the disease and, in particular, interrupting or reversing the progress of the disease. The desired reaction in a treatment of a disease may also be a delay of the onset or a prevention of the onset of the disease. An effective amount described herein will depend on the condition to be treated, the severeness of the disease, the individual parameters of the patient, including age, physiological condition, size, and weight, the duration of treatment, the type of an accompanying therapy (if present), the specific route of administration, and similar factors. Accordingly, the doses may depend on various of such parameters. In case that a reaction in the patient is insufficient with an initial dose, higher doses (or effectively higher doses achieved by a different, more localized route of administration) may be used. The disease described herein is cancer, preferably lung cancer.

In the context of the present invention, the term “kit of parts (in short: kit)” is understood to be any combination of at least some of the components identified herein, which are combined, coexisting spatially, to a functional unit, and which can contain further components.

### EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

The present inventors identified response and survival predictive miRNAs from whole blood for cancer therapy, in particular immunotherapy. These new miRNAs allow a quick and an accurate clinical prediction and survival prognosis in cancer diseases. Said miRNAs can, thus, be used as companion or complementary diagnostics in cancer therapy, in particular, immunotherapy. In addition, these new miRNAs allow to select a patient suffering from cancer to benefit from immunochemotherapy or immunotherapy.

Thus, in a first aspect, the present invention relates to a (an) (in vitro) method of predicting a response to cancer therapy of a patient suffering from cancer comprising the step of: determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Specifically, the level of at least one miRNA having
(i) a nucleotide sequence according to SEQ ID NO: 1 to SEQ ID NO: 44,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in a blood sample of a patient suffering from cancer.

In one preferred embodiment, the at least one miRNA has a nucleotide sequence selected from the group consisting of SEQ ID NO: 1 to SEQ ID NO: 8, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. In one another preferred embodiment, the at least one miRNA has a nucleotide sequence selected from the group consisting of SEQ ID NO: 1, SEQ ID NO: 7, SEQ ID NO: 13, SEQ ID NO: 34, SEQ ID NO: 39, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. FIG. 9 shows preferred combinations of the miRNAs having a nucleotide sequence according to SEQ ID NO: 1, SEQ ID NO: 7, SEQ ID NO: 13, SEQ ID NO: 34, and SEQ ID NO: 39. It should be noted that in cases where the level of at least two miRNAs is determined, said miRNAs are designated as miRNA set or signature. More preferably, the miRNA set or signature is a set or signature as comprised in Table 2. The combination of the miRNAs having a nucleotide sequence according to SEQ ID NO: 1, SEQ ID NO: 7, SEQ ID NO: 13, SEQ ID NO: 34, and SEQ ID NO: 39 is most preferred.

The level of the at least one miRNA is associated with or correlated to response prediction.

In one embodiment, the level of the at least one miRNA is compared to a reference level of said at least one miRNA.

In particular, the reference level is the level of the at least one miRNA determined empirically by measuring a number of reference blood samples from subjects suffering from cancer who are non-responders to therapy of said cancer. For example, the reference level is determined from at least 2, at least 10, at least 50, at least 100, at least 200, at least 300, at least 400, at least 500, at least 1000, at least 1500, at least 2000 at least 5000 reference samples from subjects suffering from cancer who are non-responders to therapy of said cancer.

In one preferred embodiment,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto below the reference level indicates that the patient will respond to said cancer therapy,
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto above the reference level indicates that the patient will respond to said cancer therapy,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or above the reference level indicates that the patient will not respond to said cancer therapy, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or below the reference level indicates that the patient will not respond to said cancer therapy.

Additionally, or alternatively, the reference level is the level of the at least one miRNA determined empirically by measuring a number of reference blood samples from subjects suffering from cancer who are responders to therapy of said cancer. For example, the reference level is determined from at least 2, at least 10, at least 50, at least 100, at least 200, at least 300, at least 400, at least 500, at least 1000, at least 1500, at least 2000 at least 5000 reference samples from subjects suffering from cancer who are responders to therapy of said cancer.

In one preferred embodiment,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto above the reference level indicates that the patient will not respond to said therapy,
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto below the reference level indicates that the patient will not respond to said therapy,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or below the reference level indicates that the patient will respond to said therapy, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or above the reference level indicates that the patient will respond to said therapy.

It is practicable to take one reference blood sample per subject for analysis. Said reference level may be an average reference level. It may be determined by measuring reference levels of subjects and calculating the “average” value (e.g. mean, median or modal value) thereof. Said reference level may also be determined by the weighted sum of the level of one or more miRNAs (preferably by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto). It is preferred that the reference blood sample is from the same source (e.g. whole blood or blood fraction) than the blood sample of the patient. It is further preferred that the reference level is of a subject of the same gender (e.g. female or male) and/or of a similar age/phase of life (e.g. adults or elderly) than the patient to be tested.

In the context of the present invention, said “reference level” is sometimes referred to as “miRisk”. In an exemplary embodiment, the “reference level determined by the weighted sum of the level of one or more RNAs” is calculated according to the following formula:

miRisk=(log(miR-2115-3p RPM+1)×1.870)+(log(miR-218-5p RPM+1)×0.907)+(log(miR-224-5p RPM+1)×0.495)+(log(miR-4676-3p RPM+1)×1.309)+(log(miR-6503-5p RPM+1)×1.159).

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

In a particular embodiment, subjects with miRisk scores above this miRisk score threshold are non-responders and subjects with miRisk scores below this miRisk score threshold are responders to therapy.

In a particular exemplary embodiment, an miRisk score threshold is about −0.139; i.e. subjects with miRisk scores above this miRisk score threshold are non-responders, while subjects with miRisk scores below this miRisk score threshold are responders to therapy.

Preferably, the level of the at least one miRNA is at least 0.6-fold or 0.7-fold, more preferably at least 0.8-fold or 0.9-fold, even more preferably at least 1.2-fold, 1.5-fold, or 2.0-fold, and still even more preferably at least 3.0-fold or 4.0-fold below/above the reference level. For example, the level of the at least one miRNA is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, or at least 4.0-fold below/above the reference level.

“Comparable”, as mentioned above, preferably means that the level varies between 0 and <20%, e.g. 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 19.9, 19.99, or 19.999%. “Comparable” in this respect specifically means that the detected level variation is within the accuracy of a measurement. The accuracy of a measurement depends on the measurement method used.

In one alternative embodiment, the level of the at least one miRNA is compared to an empirically determined (median) cut-off score, (median) cut-off level, or (median) threshold level. Specifically, the (median) cut-off score is determined by the weighted sum of one or more miRNAs (preferably by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto). More specifically, the (median) cut-off score allows to classify/stratify the patient as patient who will respond to cancer therapy or as a patient who will not respond to cancer therapy. As to the specific formula, it is referred to the above.

In one another embodiment, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy.

Preferably, the cancer therapy is immunotherapy or chemotherapy.

More preferably, the immunotherapy is accompanied with the administration of at least one immune checkpoint inhibitor. Even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

More preferably, the chemotherapy is accompanied with the administration of at least one chemotherapeutic agent. Even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy.

Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

If it is revealed that the patient will respond to the cancer therapy, the patient can be assigned to the cancer therapy and subsequently treated with said cancer therapy.

In one more preferred embodiment, the method predicts a response of a patient suffering from cancer to immunotherapy, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one even more preferred embodiment, the method predicts a response of a patient suffering from cancer to immunotherapy accompanied with the administration of at least one immune checkpoint inhibitor, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one still even more preferred embodiment, the method predicts a response of a patient suffering from lung cancer, preferably non-small-cell lung carcinoma (NSCLC), breast cancer, cervical cancer, gastric cancer, or bladder cancer to immunotherapy, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from lung cancer, preferably non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one most preferred embodiment, the method predicts a response of a patient suffering from lung cancer, particularly non-small-cell lung carcinoma (NSCLC), breast cancer, cervical cancer, gastric cancer, or bladder cancer to immunotherapy accompanied with the administration of at least one immune checkpoint inhibitor, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from lung cancer, particularly non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

The miRNA biomarkers of the present invention could be used as alternative biomarkers for the PD-L1 biomarker in immunotherapy. PD-L1 is a biomarker of response to immune-checkpoint inhibitors. The experimental data of the present invention show that the miRNA biomarkers of the present invention allow to predict a response of patients suffering from lung cancer to immunotherapy, in particular with immune checkpoint inhibitors. The miRNA biomarkers of the present invention could also complement the PD-L1 biomarker. The mainstay of response prediction to immunotherapies in the PD-1 inhibitor class is the quantification of tumor PD-L1 expression. However, still only approximately 30% of patients will achieve a positive response. The combinatory use of the miRNA biomarkers of the present invention with the PD-L1 biomarker known in the prior art may improve/increase the value of 30%.

In an alternative aspect, the present invention relates to a method of predicting a response to cancer therapy of a patient suffering from cancer comprising the step of:
determining the level of at least one miRNA, wherein the at least miRNA specifically occurs in myeloid cells.

Myeloid cells are a subgroup of leukocytes. Myeloid cells encompass granulocytes, monocytes, macrophages, and dendritic cells (DCs). Specifically, the myeloid cells are selected from the group consisting of granulocytes, monocytes, macrophages, and dendritic cells (DCs), or are a combination/mixture thereof.

Preferably, the level of at least one miRNA (e.g. 1, 2, 3, 4, 5, 6, 7, 8, 9, 10, 11, 12, 13, 14, or 15 miRNA(s)) is determined in a blood sample of a patient suffering from cancer, wherein the at least one miRNA has a nucleotide sequence selected from the group consisting of SEQ ID NO: 1, SEQ ID NO: 5, SEQ ID NO: 7, SEQ ID NO: 8, SEQ ID NO: 12, SEQ ID NO: 17, SEQ ID NO: 19, SEQ ID NO: 28, SEQ ID NO: 31, SEQ ID NO: 34, SEQ ID NO: 38, SEQ ID NO: 41 to SEQ ID NO: 44, a fragment thereof, and a sequence having at least 80%, more preferably at least 85%, even more preferably at least 90%, and still even more preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

As to the other preferred embodiments, it is referred to the first aspect of the present invention.

In a second aspect, the present invention relates to a (an) (in vitro) method of providing a survival prognosis to a patient suffering from cancer comprising the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80% sequence identity thereto.

Specifically, the level of at least one miRNA having
(i) a nucleotide sequence according to SEQ ID NO: 1 to SEQ ID NO: 44,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in a blood sample of a patient suffering from cancer.

In one preferred embodiment, the at least one miRNA has a nucleotide sequence selected from the group consisting of SEQ ID NO: 1 to SEQ ID NO: 8, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. In one another preferred embodiment, the at least one miRNA has a nucleotide sequence selected from the group consisting of SEQ ID NO: 1, SEQ ID NO: 7, SEQ ID NO: 13, SEQ ID NO: 34, SEQ ID NO: 39, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. FIG. 9 shows preferred combinations of the miRNAs having a nucleotide sequence according to SEQ ID NO: 1, SEQ ID NO: 7, SEQ ID NO: 13, SEQ ID NO: 34, and SEQ ID NO: 39. It should be noted that in cases where the level of at least two miRNAs is determined, said miRNAs are designated as miRNA set or signature. More preferably, the miRNA set or signature is a set or signature as comprised in Table 2. The combination of the miRNAs having a nucleotide sequence according to SEQ ID NO: 1, SEQ ID NO: 7, SEQ ID NO: 13, SEQ ID NO: 34, and SEQ ID NO: 39 is most preferred.

The level of the at least one miRNA is associated with or correlated to a survival prognosis.

In one embodiment, the level of the at least one miRNA is compared to a reference level of said at least one miRNA.

In particular, the reference level is the level of the at least one miRNA determined empirically by measuring a number of reference blood samples from subjects suffering from cancer having a known poor survival prognosis. For example, the reference level is determined from at least 2, at least 10, at least 50, at least 100, at least 200, at least 300, at least 400, at least 500, at least 1000, at least 1500, at least 2000 at least 5000 reference samples from subjects suffering from cancer having a known poor survival prognosis.

In one preferred embodiment
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto below the reference level indicates that the patient has a good survival prognosis,
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto above the reference level indicates that the patient has a good survival prognosis,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or above the reference level indicates that the patient has a poor survival prognosis, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or below the reference level indicates that the patient has a poor survival prognosis.

Additionally, or alternatively, the reference level is the level of the at least one miRNA determined empirically by measuring a number of reference blood samples from subjects suffering from cancer having a known good survival prognosis. For example, the reference level is determined from at least 2, at least 10, at least 50, at least 100, at least 200, at least 300, at least 400, at least 500, at least 1000, at least 1500, at least 2000 at least 5000 reference samples from subjects suffering from cancer having a known good survival prognosis.

In one preferred embodiment
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80% sequence, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, identity thereto above the reference level indicates that the patient has a poor survival prognosis,
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto below the reference level indicates that the patient has a poor survival prognosis,
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or below the reference level indicates that the patient has a good survival prognosis, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto comparable with or above the reference level indicates that the patient has a good survival prognosis.

It is practicable to take one reference blood sample per subject for analysis. Said reference level may be an average reference level. It may be determined by measuring reference levels of subjects and calculating the “average” value (e.g. mean, median or modal value) thereof. Said reference level may also be determined by the weighted sum of the level of one or more miRNAs (preferably by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto). It is preferred that the reference blood sample is from the same source (e.g. whole blood or blood fraction) than the blood sample of the patient. It is further preferred that the reference level is of a subject of the same gender (e.g. female or male) and/or of a similar age/phase of life (e.g. adults or elderly) than the patient to be tested.

In the context of the present invention, said “reference level” is sometimes referred to as “miRisk”. In an exemplary embodiment, the “reference level determined by the weighted sum of the level of one or more RNAs” is calculated according to the following formula:

$$miRisk = ( log ( miR - 2115 - 3 p RPM + 1 ) \times 1.87 ) + ( log ( miR - 218 - 5 p RPM + 1 ) \times 0.907 ) + ( log ( miR - 224 - 5 p RPM + 1 ) \times 0.495 ) + ( log ( miR - 4676 - 3 p RPM + 1 ) \times 1.309 ) + ( log ( miR - 6503 - 5 p RPM + 1 ) \times 1.159 ) .$$

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

In a particular embodiment, subjects with miRisk scores above this miRisk score threshold have a poor survival prognosis and subjects with miRisk scores below this miRisk score threshold have a good survival prognosis.

In a particular exemplary embodiment, the miRisk score is about −0.139; i.e. subjects with miRisk scores above this miRisk score threshold have a poor survival prognosis, while subjects with miRisk scores below this miRisk score threshold have a good survival prognosis.

Preferably, the level of the at least one miRNA is at least 0.6-fold or 0.7-fold, more preferably at least 0.8-fold or 0.9-fold, even more preferably at least 1.2-fold, 1.5-fold, or 2.0-fold, and still even more preferably at least 3.0-fold or 4.0-fold below/above the reference level. For example, the level of the at least one miRNA is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, or at least 4.0-fold below/above the reference level.

“Comparable”, as mentioned above, preferably means that the level varies between 0 and <20%, e.g. 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 19.9, 19.99, or 19.999%. “Comparable” in this respect specifically means that the detected level variation is within the accuracy of a measurement. The accuracy of a measurement depends on the measurement method used.

In one alternative embodiment, the level of the at least one miRNA is compared to an empirically determined (median) cut-off score, (median) cut-off level, or (median) threshold level. Specifically, the (median) cut-off score is determined by the weighted sum of one or more miRNAs (preferably by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto). More specifically, the (median) cut-off score allows to classify the patient as having a good or poor survival prognosis. As to the specific formula, it is referred to the above.

Specifically,
the good survival prognosis is associated with a high chance/probability of surviving a certain time period, or
the poor survival prognosis is associated with a low chance/probability of surviving a certain time period.

More specifically,
the patient having a good prognosis has a chance/probability of greater than 50%, preferably of greater than 60%, more preferably of greater than 70%, even more preferably of greater than 80%, still even more preferably of greater than 90%, or most preferably of greater than 95% to survive a certain time period, or
the patient having a low prognosis has a chance/probability of equal to or lower than 50%, preferably lower 40%, more preferably of lower than 30%, even more preferably of lower than 20%, still even more preferably of lower than 10%, and most preferably of lower than 5% to survive a certain time period.

Even more specifically, the certain time period is a time period of (at least/at most) 3 months, 6 months, 9 months, 12 months (1 year), 2 years, 3 years, 4 years, 5 years, 6 years, 7 years, 8 years, 9 years, or 10 years.

For example, the patient having a good prognosis has a chance/probability of greater than 50%, preferably of greater than 60%, more preferably of greater than 70%, even more preferably of greater than 80%, still even more preferably of greater than 90%, or most preferably of greater than 95% to survive a time period of (at least) 3 months, 6 months, 9 months, 12 months (1 year), 2 years, 3 years, 4 years, 5 years, 6 years, 7 years, 8 years, 9 years, or 10 years, or the patient having a low prognosis has a chance/probability of equal to or lower than 50%, preferably lower 40%, more preferably of lower than 30%, even more preferably of lower than 20%, still even more preferably of lower than 10%, and most preferably of lower than 5% to survive a time period of (at most) 3 months, 6 months, 9 months, 12 months (1 year), 2 years, 3 years, 4 years, 5 years, 6 years, 7 years, 8 years, 9 years, or 10 years.

The survival prognosis may also be defined as the predicted survival at 2- or 5-years follow-up. For example, a patient having a good survival prognosis will still be alive at a 2- or 5-years follow-up, while a patient having a poor survival prognosis will not be alive at a 2- or 5-years follow up anymore.

In one another embodiment, the patient is a patient to whom a cancer therapy will be, is, has been administered. Preferably, the patient is a treatment naïve cancer patient. If it is revealed that the patient has a good survival prognosis, the patient can be assigned to the cancer therapy and subsequently treated with said cancer therapy. Preferably, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy. More preferably, the cancer therapy is immunotherapy or chemotherapy.

Even more preferably, the immunotherapy is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

Even more preferably, the chemotherapy is accompanied with the administration of at least one chemotherapeutic agent. Still even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy.

Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

In one more preferred embodiment, the method provides a survival prognosis to a patient suffering from cancer, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
wherein the patient is a patient to whom an immunotherapy will be, is, or has been administered.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one even more preferred embodiment, the method provides a survival prognosis to a patient suffering from cancer, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
wherein the patient is a patient to whom an immunotherapy accompanied with the administration of at least one immune checkpoint inhibitor will be, is, or has been administered.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one still even more preferred embodiment, the method provides a survival prognosis to a patient suffering from lung cancer, preferably non-small-cell lung carcinoma (NSCLC), breast cancer, cervical cancer, gastric cancer, or bladder cancer, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from lung cancer, preferably non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
wherein the patient is a patient to whom an immunotherapy will be, is, or has been administered.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one most preferred embodiment, the method provides a survival prognosis to a patient suffering from lung cancer, preferably non-small-cell lung carcinoma (NSCLC), breast cancer, cervical cancer, gastric cancer, or bladder cancer, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from lung cancer, preferably non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
wherein the patient is a patient to whom an immunotherapy accompanied with the administration of at least one immune checkpoint inhibitor will be, is, or has been administered.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

The miRNA biomarkers of the present invention could be used as alternative biomarkers for the PD-L1 biomarker in immunotherapy. The experimental data of the present invention show that the miRNA biomarkers of the present invention allow to make a survival prognosis of patients suffering from lung cancer who received an immunotherapy, in particular with immune checkpoint inhibitors. The miRNA biomarkers of the present invention could also complement the PD-L1 biomarker.

In an alternative aspect, the present invention relates to a method of providing a survival prognosis to a patient suffering from cancer comprising the step of:
determining the level of at least one miRNA, wherein the at least miRNA specifically/exclusively occurs in myeloid cells.

Myeloid cells are a subgroup of leukocytes. Myeloid cells encompass granulocytes, monocytes, macrophages, and dendritic cells (DCs). Specifically, the myeloid cells are selected from the group consisting of granulocytes, monocytes, macrophages, and dendritic cells (DCs), or are a combination/mixture thereof.

Preferably, the level of at least one miRNA (e.g. 1, 2, 3, 4, 5, 6, 7, 8, 9, 10, 11, 12, 13, 14, or 15 miRNA(s)) is determined in a blood sample of a patient suffering from cancer, wherein the at least one miRNA has a nucleotide sequence selected from the group consisting of SEQ ID NO: 1, SEQ ID NO: 5, SEQ ID NO: 7, SEQ ID NO: 8, SEQ ID NO: 12, SEQ ID NO: 17, SEQ ID NO: 19, SEQ ID NO: 28, SEQ ID NO: 31, SEQ ID NO: 34, SEQ ID NO: 38, SEQ ID NO: 41 to SEQ ID NO: 44, a fragment thereof, and a sequence having at least 80%, more preferably at least 85%, even more preferably at least 90%, and still even more preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

As to the other preferred embodiments, it is referred to the second aspect of the present invention.

In a third aspect, the present invention relates to a (an) (in vitro) method of predicting side effects in cancer therapy of a patient comprising the step of:
determining the level of at least one miRNA in a blood sample of a patient suffering from cancer, wherein the at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Specifically, the level of at least one miRNA having
(i) a nucleotide sequence according to SEQ ID NO: 1 to SEQ ID NO: 44,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iv) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in a blood sample of a patient suffering from cancer.

In one preferred embodiment, the at least one miRNA has a nucleotide sequence selected from the group consisting of SEQ ID NO: 1 to SEQ ID NO: 8, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. In one another preferred embodiment, the at least one miRNA has a nucleotide sequence selected from the group consisting of SEQ ID NO: 1, SEQ ID NO: 7, SEQ ID NO: 13, SEQ ID NO: 34, SEQ ID NO: 39, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. FIG. 9 shows preferred combinations of the miRNAs having a nucleotide sequence according to SEQ ID NO: 1, SEQ ID NO: 7, SEQ ID NO: 13, SEQ ID NO: 34, and SEQ ID NO: 39. It should be noted that in cases where the level of at least two miRNAs is determined, said miRNAs are designated as miRNA set or signature. More preferably, the miRNA set or signature is a set or signature as comprised in Table 2. The combination of the miRNAs having a nucleotide sequence according to SEQ ID NO: 1, SEQ ID NO: 7, SEQ ID NO: 13, SEQ ID NO: 34, and SEQ ID NO: 39 is most preferred.
The level of the at least one miRNA is associated with the probability that side effects occur during cancer therapy.

In one embodiment, the level of the at least one miRNA is compared to an empirically determined (median) cut-off score, (median) cut-off level, or (median) threshold level. Specifically, the (median) cut-off score is determined by the weighted sum of the level of one or more miRNAs (preferably by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto). More specifically, the (median) cut-off score allows to classify the patient as a patient who will not suffer from side effects or as a patient who will suffer from side effects.

In the context of the present invention, said “reference level” or “cut-off score” is sometimes referred to as “miRisk”.

In an exemplary embodiment, the “the weighted sum of the level of one or more RNAs” is calculated according to the following formula:

$$miRisk = ( log ( miR - 2115 - 3 p RPM + 1 ) \times 1.87 ) + ( log ( miR - 218 - 5 p RPM + 1 ) \times 0.907 ) + ( log ( miR - 224 - 5 p RPM + 1 ) \times 0.495 ) + ( log ( miR - 4676 - 3 p RPM + 1 ) \times 1.309 ) + ( log ( miR - 6503 - 5 p RPM + 1 ) \times 1.159 ) .$$

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

In a particular embodiment, this miRisk score threshold allows to classify the patient as a patient who will not suffer from side effects or as a patient who will suffer from side effects.

In a particular exemplary embodiment, the miRisk score threshold is about −0.139; i.e. this miRisk score threshold allows to classify the patient as a patient who will not suffer from side effects or as a patient who will suffer from side effects.

Particularly, the side effects are immune-related adverse events. More particularly, the side effects, specifically immune-related adverse events, are selected from the group consisting of endocrinopathy, dermatitis, colitis, polymyaglia rheumatica (PMR), hypophisitis, myositis, thyroiditis, pneumonitis, polyarthritis, hepatitis, serositis, hypothyroidism, arthritis, synovialitides, and psoriasis.

In one another embodiment, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy.

Preferably, the cancer therapy is immunotherapy or chemotherapy.

More preferably, the immunotherapy is accompanied with the administration of at least one immune checkpoint inhibitor. Even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

More preferably, the chemotherapy is accompanied with the administration of at least one chemotherapeutic agent. Even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

The patient is a patient suffering from cancer. In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy. Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

In one more preferred embodiment, the method predicts side effects in immunotherapy of a patient, said method comprises the step of:
determining the level of at least one miRNA in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one even more preferred embodiment, the method predicts side effects in immunotherapy accompanied with the administration of at least one immune checkpoint inhibitor of a patient, said method comprises the step of:
determining the level of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) in a blood sample of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one still even more preferred embodiment, the method predicts side effects in immunotherapy of a patient, said method comprises the step of:
determining the level of at least one miRNA in a blood sample of a patient suffering from lung cancer, preferably non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one most preferred embodiment, the method predicts side effects in immunotherapy accompanied with the administration of at least one immune checkpoint inhibitor of a patient, said method comprises the step of:
determining the level of at least one miRNA in a blood sample of a patient suffering from lung cancer, preferably non-small-cell lung carcinoma (NSCLC), breast cancer, cervical cancer, gastric cancer, or bladder cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

The above-mentioned side effects are particularly immune-related adverse events. More particularly, the side effects, specifically immune-related adverse events, are selected from the group consisting of endocrinopathy, dermatitis, colitis, polymyaglia rheumatica (PMR), hypophisitis, myositis, thyroiditis, pneumonitis, polyarthritis, hepatitis, serositis, hypothyroidism, arthritis, synovialitides, and psoriasis.

In an alternative aspect, the present invention relates to a method of predicting side effects in cancer therapy of a patient comprising the step of:
determining the level of at least one miRNA, wherein the at least miRNA specifically/exclusively occurs in myeloid cells.

Myeloid cells are a subgroup of leukocytes. Myeloid cells encompass granulocytes, monocytes, macrophages, and dendritic cells (DCs). Specifically, the myeloid cells are selected from the group consisting of granulocytes, monocytes, macrophages, and dendritic cells (DCs), or are a combination/mixture thereof.

Preferably, the level of at least one miRNA (e.g. 1, 2, 3, 4, 5, 6, 7, 8, 9, 10, 11, 12, 13, 14, or 15 miRNA(s)) is determined in a blood sample of a patient suffering from cancer, wherein the at least one miRNA has a nucleotide sequence selected from the group consisting of SEQ ID NO: 1, SEQ ID NO: 5, SEQ ID NO: 7, SEQ ID NO: 8, SEQ ID NO: 12, SEQ ID NO: 17, SEQ ID NO: 19, SEQ ID NO: 28, SEQ ID NO: 31, SEQ ID NO: 34, SEQ ID NO: 38, SEQ ID NO: 41 to SEQ ID NO: 44, a fragment thereof, and a sequence having at least 80%, more preferably at least 85%, even more preferably at least 90%, and still even more preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

As to the other preferred embodiments, it is referred to the third aspect of the present invention.

In a fourth aspect, the present invention relates to a (an) (in vitro) method of monitoring a patient suffering from cancer comprising the step of:
determining the level of at least one miRNA in a blood sample of a patient suffering from cancer, wherein the at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

This method allows to determine the course of cancer in a patient.

Specifically, the level of at least one miRNA having
(i) a nucleotide sequence according to SEQ ID NO: 1 to SEQ ID NO: 44,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(v) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in a blood sample of a patient suffering from cancer.

In one preferred embodiment, the at least one miRNA has a nucleotide sequence selected from the group consisting of SEQ ID NO: 1 to SEQ ID NO: 8, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. In one another preferred embodiment, the at least one miRNA has a nucleotide sequence selected from the group consisting of SEQ ID NO: 1, SEQ ID NO: 7, SEQ ID NO: 13, SEQ ID NO: 34, SEQ ID NO: 39, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. FIG. 9 shows preferred combinations of the miRNAs having a nucleotide sequence according to SEQ ID NO: 1, SEQ ID NO: 7, SEQ ID NO: 13, SEQ ID NO: 34, and SEQ ID NO: 39. It should be noted that in cases where the level of at least two miRNAs is determined, said miRNAs are designated as miRNA set or signature. More preferably, the miRNA set or signature is a set or signature as comprised in Table 2. The combination of the miRNAs having a nucleotide sequence according to SEQ ID NO: 1, SEQ ID NO: 7, SEQ ID NO: 13, SEQ ID NO: 34, and SEQ ID NO: 39 is most preferred.

In one embodiment, the level of the at least one miRNA is compared to a reference level of said at least one miRNA.

This comparison allows to determine the course of cancer in the patient. It may be determined that the cancer worsens in the patient, that the cancer does not worsen/is stable in the patient, or that the cancer improves in the patient.

The reference level may be any level which allows to determine the course of cancer in the patient. It may be obtained from (control) subjects (i.e. subjects different from the patient to be tested such as healthy subjects and/or subjects having cancer) or from the same patient. In the latter case, the individual may be retested for cancer, e.g. in the form of a longitudinal monitoring. It may be determined that the individual is still affected by a cancer disease or not affected by a cancer disease anymore.

In the context of the present invention, said “reference level” is sometimes referred to as “miRisk”.

In an exemplary embodiment, the reference level can be calculated according to the following formula:

$$miRisk = ( log ( miR ‐ 2115 ‐ 3 p RPM + 1 ) \times 1.87 ) + ( log ( miR ‐ 218 ‐ 5 p RPM + 1 ) \times 0.907 ) + ( log ( miR ‐ 224 ‐ 5 p \\ RPM + 1 ) \times 0.495 ) + ( log ( miR ‐ 4676 ‐ 3 p RPM + 1 ) \times 1.309 ) + ( log ( miR ‐ 6503 ‐ 5 p RPM + 1 ) \times 1.159 ) .$$

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

In one particular embodiment, this miRisk score threshold may allow to determine whether the cancer worsens in the patient, whether the cancer does not worsen/is stable in the patient, or whether the cancer improves in the patient.

In a particular exemplary embodiment, the miRisk score threshold is about −0.139. This miRisk score threshold may allow to determine whether the cancer worsens in the patient, whether the cancer does not worsen/is stable in the patient, or whether the cancer improves in the patient.

In particular, the reference level is the level of the at least one miRNA determined empirically by measuring a number of reference blood samples from subjects suffering from cancer. For example, the reference level is determined from at least 2, at least 10, at least 50, at least 100, at least 200, at least 300, at least 400, at least 500, at least 1000, at least 1500, at least 2000 at least 5000 reference samples from subjects suffering from cancer.

In one another embodiment, said determining comprises determining the level of the at least one miRNA in a blood sample of a patient suffering from cancer at a first point in time and in at least one further blood sample (of the same patient) at a later point in time and comparing said levels determined at the different time points.

This comparison allows to determine the course of cancer in the patient. It may be determined that the cancer worsens in the patient, that the cancer does not worsen/is stable in the patient, or that the cancer improves in the patient.

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, or at least 2 months. For example, the patient may be routinely checked, e.g. every day or one times per week. The patient may be (re)tested at 2, 3, 4, 5, 6 7, 8, 9, or 10 time points (first point in time and further point(s) in time).

In addition to the determination of the course of cancer in the patient, the cancer therapy of the patient can be monitored. Thus, in one preferred embodiment, the patient is a patient who receives, has received, or had received a therapy of said cancer.

For example, the patient may receive a therapy during the complete determination/monitoring process (e.g. the administration of a drug or agent) or may receive a therapy before, at, or after a first point in time (e.g. the administration of a drug or agent) and may be retested at a later point in time. In particular, said first point in time may be before the initiation of a therapy and said later point in time may be during the therapy and/or after the therapy. If the therapy encompasses the administration of a drug or agent and the patient responds to said therapy, the drug or agent administration may be continued, the dose of the drug or agent may be reduced, or the drug or agent administration may be stopped. If the therapy encompasses the administration of a drug or agent and the patient does not respond to said therapy, the dose of the drug or agent may be increased, the drug or agent may be changed, or the therapy mode may be changed, e.g. immunotherapy to chemotherapy. The drug or agent may be an immune checkpoint inhibitor or a chemotherapeutic agent.

Preferably, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy. More preferably, the cancer therapy is immunotherapy or chemotherapy.

Even more preferably, the immunotherapy is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

Even more preferably, the chemotherapy is accompanied with the administration of at least one chemotherapeutic agent. Still even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy.

Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

In one more preferred embodiment, the method is for monitoring a patient suffering from cancer, said method comprises the steps of
(i) determining the level of at least one miRNA in a blood sample of a patient suffering from cancer at a first point in time and in at least one further blood sample (of the same patient) at a later point in time, and
(ii) comparing said levels determined at the different time points,
wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and
wherein the patient receives, has received, or had received an immunotherapy of said cancer.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one even more preferred embodiment, the method is for monitoring a patient suffering from cancer, said method comprises the steps of:
(i) determining the level of at least one miRNA in a blood sample of a patient suffering from cancer at a first point in time and in at least one further blood sample (of the same patient) at a later point in time, and
(ii) comparing said levels determined at the different time points,
wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and
wherein the patient receives, has received, or had received an immunotherapy of said cancer accompanied with the administration of at least one immune checkpoint inhibitor.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one still even more preferred embodiment, the method is for monitoring a patient suffering from lung cancer, preferably non-small-cell lung carcinoma (NSCLC), breast cancer, cervical cancer, gastric cancer, or bladder cancer, said method comprises the steps of
(i) determining the level of at least one miRNA in a blood sample of a patient suffering from lung cancer, preferably non-small-cell lung carcinoma (NSCLC), breast cancer, cervical cancer, gastric cancer, or bladder cancer at a first point in time and in at least one further blood sample (of the same patient) at a later point in time, and
(ii) comparing said levels determined at the different time points,
wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and
wherein the patient receives, has received, or had received an immunotherapy of said cancer.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one most preferred embodiment, the method is for monitoring a patient suffering from lung cancer, preferably non-small-cell lung carcinoma (NSCLC), breast cancer, cervical cancer, gastric cancer, or bladder cancer, said method comprises the steps of:
(i) determining the level of at least one miRNA in a blood sample of a patient suffering from lung cancer, preferably non-small-cell lung carcinoma (NSCLC), breast cancer, cervical cancer, gastric cancer, or bladder cancer at a first point in time and in at least one further blood sample (of the same patient) at a later point in time, and
(ii) comparing said levels determined at the different time points,
wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and
wherein the patient receives, has received, or had received an immunotherapy accompanied with the administration of at least one immune checkpoint inhibitor of said cancer.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

The above method, thus, allows the serially assessment of the disease status for evidence of an effect of a cancer therapy.

In an alternative aspect, the present invention relates to a method of monitoring a patient suffering from cancer comprising the step of:
determining the level of at least one miRNA, wherein the at least miRNA specifically/exclusively occurs in myeloid cells.

Myeloid cells are a subgroup of leukocytes. Myeloid cells encompass granulocytes, monocytes, macrophages, and dendritic cells (DCs). Specifically, the myeloid cells are selected from the group consisting of granulocytes, monocytes, macrophages, and dendritic cells (DCs), or are a combination thereof.

Preferably, the level of at least one miRNA (e.g. 1, 2, 3, 4, 5, 6, 7, 8, 9, 10, 11, 12, 13, 14, or 15 miRNA(s)) is determined in a blood sample of a patient suffering from cancer, wherein the at least one miRNA has a nucleotide sequence selected from the group consisting of SEQ ID NO: 1, SEQ ID NO: 5, SEQ ID NO: 7, SEQ ID NO: 8, SEQ ID NO: 12, SEQ ID NO: 17, SEQ ID NO: 19, SEQ ID NO: 28, SEQ ID NO: 31, SEQ ID NO: 34, SEQ ID NO: 38, SEQ ID NO: 41 to SEQ ID NO: 44, a fragment thereof, and a sequence having at least 80%, more preferably at least 85%, even more preferably at least 90%, and still even more preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

As to the other preferred embodiments, it is referred to the fourth aspect of the present invention.

In the methods of the first to fourth aspect, the blood sample is preferably whole blood or a blood fraction. In particular the blood fraction is selected from the group consisting of a blood cell fraction and plasma or serum. More preferably, the blood fraction is selected from the group consisting of a blood cell fraction, plasma, and serum. The blood cell fraction may comprise erythrocytes, leukocytes, and/or thrombocytes. Even more preferably, the blood cell fraction is a fraction of leukocytes. Specifically, the leukocytes are myeloid cells and/or lymphocytes. More specifically, the myeloid cells are selected from the group consisting of granulocytes, monocytes, macrophages, and dendritic cells (DCs), or are a combination thereof.

In the methods of the first to fourth aspect, the level is preferably determined by sequencing, more preferably next generation sequencing, nucleic acid hybridization, nucleic acid amplification, polymerase extension, mass spectrometry or any combination thereof. Specifically,
the level is the expression level.

In a fifth aspect, the present invention relates to a (an) (in vitro) method of determining whether to treat a patient suffering from cancer comprising the steps of.
(i) carrying out the method according to the first aspect, thereby identifying the patient as patient who will respond to cancer therapy, carrying out the method according to the second aspect, thereby identifying the patient as patient having a good survival prognosis, or carrying out the method according to the third aspect, thereby identifying the patient as patient who will not suffer from side effects in cancer therapy, and
(ii) assigning the patient to (said) cancer therapy.

In one embodiment, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy.

Preferably, the cancer therapy is immunotherapy or chemotherapy.

More preferably, the immunotherapy is accompanied with the administration of at least one immune checkpoint inhibitor. Even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

More preferably, the chemotherapy is accompanied with the administration of at least one chemotherapeutic agent. Even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy.

Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

After having assigned the patient to the cancer therapy, the patient is finally treated with said cancer therapy.

In one more preferred embodiment, the method is for determining whether to treat a patient suffering from cancer, said method comprises the steps of.
(i) carrying out the method according to the first aspect, thereby identifying the patient as patient who will respond to immunotherapy, carrying out the method according to the second aspect, thereby identifying the patient as patient having a good survival prognosis, or carrying out the method according to the third aspect, thereby identifying the patient as patient who will not suffer from side effects in immunotherapy, and
(ii) assigning the patient to (said) immunotherapy.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one even more preferred embodiment, the method is for determining whether to treat a patient suffering from cancer, said method comprises the steps of:
(i) carrying out the method according to the first aspect, thereby identifying the patient as patient who will respond to immunotherapy accompanied with the administration of at least one immune checkpoint inhibitor, carrying out the method according to the second aspect, thereby identifying the patient as patient having a good survival prognosis, or carrying out the method according to the third aspect, thereby identifying the patient as patient who will not suffer from side effects in immunotherapy accompanied with the administration of at least one immune checkpoint inhibitor, and
(ii) assigning the patient to (said) immunotherapy.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one still even more preferred embodiment, the method is for determining whether to treat a patient suffering from lung cancer, preferably non-small-cell lung carcinoma (NSCLC), breast cancer, cervical cancer, gastric cancer, or bladder cancer, said method comprises the steps of
(i) carrying out the method according to the first aspect, thereby identifying the patient as patient who will respond to immunotherapy, carrying out the method according to the second aspect, thereby identifying the patient as patient having a good survival prognosis, or carrying out the method according to the third aspect, thereby identifying the patient as patient who will not suffer from side effects in immunotherapy, and
(ii) assigning the patient to (said) immunotherapy.

Preferably, the immunotherapy is accompanied with the administration of at least one immune checkpoint inhibitor. More preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In one most preferred embodiment, the method is for determining whether to treat a patient suffering from lung cancer, preferably non-small-cell lung carcinoma (NSCLC), breast cancer, cervical cancer, gastric cancer, or bladder cancer, said method comprises the steps of
(i) carrying out the method according to the first aspect, thereby identifying the patient as patient who will respond to immunotherapy accompanied with the administration of at least one immune checkpoint inhibitor, or carrying out the method according to the third aspect, thereby identifying a patient as patient who will not suffer from side effects in immunotherapy accompanied with the administration of at least one immune checkpoint inhibitor, and
(ii) assigning the patient to said immunotherapy.

Preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

In a sixth aspect, the present invention relates to the (in vitro) use of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) or polynucleotide allowing the detection of said at least one miRNA for predicting a response to cancer therapy of a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In a seventh aspect, the present invention relates to the (in vitro) use of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) or polynucleotide allowing the detection of said at least one miRNA for providing a survival prognosis to a patient suffering from cancer, wherein the at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In an eighth aspect, the present invention relates to the (in vitro) use of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) or polynucleotide allowing the detection of said at least one miRNA for predicting side effects in cancer therapy of a patient, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In a night aspect, the present invention relates to the (in vitro) use of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) or polynucleotide allowing the detection of said at least one miRNA for monitoring a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In a tenth aspect, the present invention relates to the (in vitro) use of at least one miRNA (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 miRNA(s)) or polynucleotide allowing the detection of said at least one miRNA for determining whether to treat a patient suffering from cancer, wherein the at least one miRNA has a nucleotide sequence selected from the group consisting of SEQ ID NO: 1 to SEQ ID NO: 44, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

With respect to the seventh to tenth aspect, the following applies:

In one embodiment, the cancer therapy is selected from the group consisting of immunotherapy, chemotherapy, radiation therapy, vaccination therapy, stem cell therapy, anti-hormonal therapy, immunosuppressive therapy, antibody therapy, and surgical therapy.

Preferably, the cancer therapy is immunotherapy or chemotherapy.

More preferably, the immunotherapy is accompanied with the administration of at least one immune checkpoint inhibitor. Even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway.

More preferably, the chemotherapy is accompanied with the administration of at least one chemotherapeutic agent. Even more preferably, the at least one chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

In one additional or alternative embodiment, the cancer is a cancer eligible for immunotherapy, chemotherapy, radiation therapy, vaccination therapy, stem cell therapy, anti-hormonal therapy, immunosuppressive therapy, antibody therapy, and/or surgical therapy.

Preferably, the cancer is eligible for immunotherapy, chemotherapy, or immunochemotherapy.

More preferably, the cancer eligible for immunotherapy is sensitive to immune checkpoint inhibition/immune checkpoint therapy. Even more preferably, immune checkpoint inhibition is accompanied with the administration of at least one immune checkpoint inhibitor. Still even more preferably, the at least one immune checkpoint inhibitor is selected from the group consisting of an inhibitor targeting PD-1, PD-L1, PD-L2, CTLA-4, LAG3, TIGIT, CD73 and intrinsic checkpoint blockades. Specifically, the at least one immune checkpoint inhibitor is an inhibitor targeting PD-1/PD-L1 or the PD-1/PD-L1 pathway. Alternatively, the cancer is a PD-L1 expressing cancer.

More preferably, the cancer eligible for chemotherapy is sensitive to a chemotherapeutic agent.

Even more preferably, the chemotherapeutic agent is selected from the group consisting of an alkylating agent, an antimetabolite, folinic acid, a folate antagonist, a mitotic inhibitor, an anthracycline, a topoisomerase inhibitor, an antibody, a signal transduction inhibitor, an inhibitor of angiogenesis, and an inhibitor of histone deacetylase.

Specifically, the cancer is selected from the group consisting of lung cancer, preferably non-small-cell lung carcinoma (NSCLC), breast cancer, cervical cancer, gastric cancer, bladder cancer, skin cancer, nasopharyngeal cancer, neuroendocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

More specifically, the cancer is selected from the group consisting of lung cancer, preferably non-small-cell lung carcinoma (NSCLC), breast cancer, cervical cancer, gastric cancer, and bladder cancer. Said cancer is eligible to immunotherapy.

The polynucleotide may be a primer (e.g. a primer probe) or a polynucleotide probe.

Preferably, the (level of the) at least one miRNA is detected/determined in a blood sample of the patient suffering from cancer. As to the preferred embodiments of the blood sample, it is referred to the first to fourth aspect of the present invention.

In an eleventh aspect, the present invention relates to (an (in vitro) use of) a kit for predicting a response to cancer therapy of a patient suffering from cancer, for providing a survival prognosis to a patient suffering from cancer, for predicting side effects in cancer therapy of a patient, for monitoring a patient suffering from cancer, or for determining whether to treat a patient suffering from cancer, wherein said kit comprises:
means for determining the level of at least one miRNA having a nucleotide sequence selected from the group consisting of SEQ ID NO: 1 to SEQ ID NO: 44, a fragment thereof, and a sequence having at least 80% sequence identity thereto.

Specifically, the kit comprises means for determining the level of at least one miRNA having
(i) a nucleotide sequence according to SEQ ID NO: 1 to SEQ ID NO: 44,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(vi) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)

In one preferred embodiment, the at least one miRNA has a nucleotide sequence selected from the group consisting of SEQ ID NO: 1 to SEQ ID NO: 8, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. In one another preferred embodiment, the at least one miRNA has a nucleotide sequence selected from the group consisting of SEQ ID NO: 1, SEQ ID NO: 7, SEQ ID NO: 13, SEQ ID NO: 34, SEQ ID NO: 39, a fragment thereof, and a sequence having at least 80%, more preferably at least 85%, even more preferably at least 90%, and still even more preferably at least 95% or 99%, e.g. 80, 81, 82, 83, 84, 85, 86, 87, 88, 89, 90, 91, 92, 93, 94, 95, 96, 97, 98, or 99%, sequence identity thereto. FIG. 9 shows preferred combinations of the miRNAs having a nucleotide sequence according to SEQ ID NO: 1, SEQ ID NO: 7, SEQ ID NO: 13, SEQ ID NO: 34, and SEQ ID NO: 39. It should be noted that in cases where the level of at least two miRNAs is determined, said miRNAs are designated as miRNA set or signature. More preferably, the miRNA set or signature is a set or signature as comprised in Table 2. The combination of the miRNAs having a nucleotide sequence according to SEQ ID NO: 1, SEQ ID NO: 7, SEQ ID NO: 13, SEQ ID NO: 34, and SEQ ID NO: 39 is most preferred.

More specifically, the kit comprises
means for carrying out next generation sequencing (NGS),
at least one polynucleotide (probe) for detecting the at least one miRNA,
at least one primer (e.g. a primer pair) for binding the at least one miRNA, and/or
at least one antibody capable of binding a hybrid of said at least one polynucleotide (probe) and said at least one miRNA.

Said means allow to determine the level of the at least one miRNA in a blood sample of a patient suffering from cancer and, thus, to predict a response to cancer therapy of the patient suffering from cancer, to provide a survival prognosis to the patient suffering from cancer, to predict side effects in cancer therapy of the patient, to monitor the patient suffering from cancer, or to determine whether to treat the patient suffering from cancer.

The at least one polynucleotide (probe) may be part of a microarray/biochip or may be attached to beads of a beads-based multiplex system. The at least one polynucleotide (primer, primer pair) may be part of a RT-PCR system, a PCR-system, or a next generation sequencing system.

Said means may further comprise a microarray, a RT-PCT system, a PCR-system, a flow cytometer, a Luminex system, and/or a next generation sequencing system.

Preferably, the (level of the) at least one miRNA is detected/determined in a blood sample of the patient suffering from cancer. As to the preferred embodiments of the blood sample, it is referred to the first to fourth aspect of the present invention.

Even more specifically, the kit comprises means for determining the level of PD-L1, in particular in a biological sample, such as a tissue sample, from a patient suffering from cancer.

Said means can encompass a polynucleotide (probe), a primer (e.g. a primer pair), and/or an antibody being PD-L1 specific.

The kit may further comprise
(iii) a container, and/or
(iv) a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise the at least one reference, e.g. the reference level of the level of the at least one miRNA determined herein or the cut-off score. In case that the data carrier comprises an access-code which allows the access to a database, said at least one reference, e.g. said reference level or cut-off score may be deposited in this database.

The data carrier may also comprise information or instructions on how to carry out the method according to the first to fifth aspect of the present invention.

The kit may also comprise materials desirable from a commercial and user standpoint including a buffer(s), a reagent(s) and/or a diluent(s) for determining the level mentioned above.

As to the first to eleventh aspect, the patient suffering from cancer is preferably a mammal, in particular a human.

In all aspects described above, the combination of the miRNAs having a nucleotide sequence according to SEQ ID NO: 1, SEQ ID NO: 7, SEQ ID NO: 13, SEQ ID NO: 34, and SEQ ID NO: 39 is most preferred.

Immunotherapy is revolutionizing the standard of care for numerous cancers. However, an important current limitation is the lack of reliable efficacy biomarkers. In advanced stage NSCLC expressing high levels of PD-L1 (PD-L1 Tumor Proportion Score (TPS)≥50%), for example, immunotherapy alone or immunotherapy in combination with chemotherapy are recommended as treatment options in major international guidelines. Nevertheless, there remains uncertainty as to the ideal therapeutic choice in individual patients. The present inventors have identified new miRNAs which allow to select a patient suffering from cancer to benefit from immunochemotherapy or immunotherapy.

Thus, in a twelfth aspect, the present invention relates to a (an) (in vitro) method of selecting a patient suffering from cancer to (likely) benefit from immunochemotherapy or immunotherapy comprising the step of:
determining the level of at least one miRNA (e.g. 1, 2, 3, 4, or 5 miRNA(s)) in a blood sample of a patient suffering from cancer,
wherein the at least one miRNA (e.g. 1, 2, 3, 4, or 5 miRNA(s)) has a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80%, preferably at least 85%, more preferably at least 90%, and even more preferably at least 95% or 99%, e.g. 80, 81, 82, 83, 84, 85, 86, 87, 88, 89, 90, 91, 92, 93, 94, 95, 96, 97, 98, or 99%, sequence identity thereto, and
wherein the patient has optionally a PD-L1 Tumor Proportion Score (TPS) of ≥50%.

It is preferred that the patient having a PD-L1 TPS of ≥50% has no oncogene activating mutations. Such oncogene activating mutations may be EGFR mutations, BRAF V660e mutations, ALK translocations, and/or ROS-1 translocations.

It is further (alternatively or additionally) preferred that the cancer is eligible for immunochemotherapy or immunotherapy. A cancer eligible for immunochemotherapy is sensitive to an immunotherapeutic agent and a chemotherapeutic agent. A cancer eligible for immunotherapy is sensitive to an immunotherapeutic agent.

Specifically, the cancer eligible for immunotherapy is sensitive to immune checkpoint inhibition.

More specifically, the immune checkpoint inhibition is accompanied with the administration of at least one immune checkpoint inhibitor. Even more specifically, the at least one immune checkpoint inhibitor is an inhibitor targeting PD-1/PD-L1 or the PD-1/PD-L1 pathway.

It is also (alternatively or additionally) preferred that the cancer is selected from the group consisting of lung cancer, breast cancer, cervical cancer, gastric cancer, bladder cancer, skin cancer, nasopharyngeal cancer, neuroendrocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

It is more preferred that the cancer is selected from the group consisting of lung cancer, breast cancer, cervical cancer, gastric cancer, skin cancer, and bladder cancer. Said cancer is eligible for immunochemotherapy or immunotherapy.

It is even more preferred that the lung cancer is non-small-cell lung carcinoma (NSCLC).

It is still even more preferred that the non-small-cell lung carcinoma (NSCLC) is late stage NSCLC. It is most preferred that the late stage NSCLC is NSCLC of stage IV.

In one embodiment, the level of the at least one miRNA is compared to a (miRisk) cut-off score. The (miRisk) cut-off score allows to classify/select a patient as a patient who will (likely) benefit from immunochemotherapy, or as a patient who will (likely) benefit from immunochemotherapy or immunotherapy.

Preferably,
the level of the at least one miRNA above the (miRisk) cut-off score indicates that the patient will (likely) benefit from immunochemotherapy, or
the level of the at least one miRNA below or equal to the (miRisk) cut-off score indicates that the patient will (likely) benefit from immunochemotherapy or immunotherapy.

In one alternative embodiment, the method further comprises the step of determining a (miRisk) score by summarizing the weighted levels of at least two, at least three, at least four, or five miRNAs having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80%, preferably at least 85%, more preferably at least 90%, and even more preferably at least 95% or 99%, e.g. 80, 81, 82, 83, 84, 85, 86, 87, 88, 89, 90, 91, 92, 93, 94, 95, 96, 97, 98, or 99%, sequence identity thereto.

Specifically, the (miRisk) score is determined by summarizing the weighted levels of five miRNAs having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), and SEQ ID NO: 34 (miR-6503-5p).

Preferably, the (miRisk) score is calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) ,$$

wherein RPM means reads per million.

In this respect, it should be noted that the miRNA having a nucleotide sequence according to SEQ ID NO: 1 is miR-2115-3p, the miRNA having a nucleotide sequence according to SEQ ID NO: 39 is miR-218-5p, the miRNA having a nucleotide sequence according to SEQ ID NO: 13 is miR-224-5p, the miRNA having a nucleotide sequence according to SEQ ID NO: 7 is miR-4676-3p, and the miRNA having a nucleotide sequence according to SEQ ID NO: 34 is miR-6503-5p.

More preferably, the (miRisk) score as defined above is compared to a (miRisk) cut-off score.

Even more preferably, the (miRisk) cut-off score is about −0.073.

Still even more preferably,
a (miRisk) score > about −0.073 indicates that the patient will (likely) benefit from immunochemotherapy, or
a (miRisk) score ≤ about −0.073 indicates that the patient will (likely) benefit from immunochemotherapy or immunotherapy.

The patient suffering from cancer is preferably a mammal, in particular a human.

As to the preferred embodiments of the blood sample, it is referred to the first to eleventh aspect of the present invention.

In a thirteenth aspect, the present invention relates to a (an) (in vitro) method of determining whether to treat a patient suffering from cancer with immunochemotherapy or immunotherapy comprising the steps of:
(i) carrying out the method according to the twelfth aspect, thereby identifying the patient as patient who will (likely) benefit from immunochemotherapy or immunotherapy, and
(ii) assigning the patient to said therapy.

As mentioned above, a patient having a (miRisk) score > about −0.073 will (likely) benefit from immunochemotherapy and is assigned to said therapy. Alternatively, a patient having a (miRisk) score ≤ about −0.073 will (likely) benefit from immunochemotherapy or immunotherapy and is assigned to one of said therapies. In this case, immunotherapy is usually chosen as it is of less therapeutic burden to the patient than immunotherapy in combination with chemotherapy.

Preferably, the immunotherapeutic agent is an inhibitor targeting PD-1/PD-L1. More preferably, the inhibitor targeting PD-1/PD-L1 is selected from the group consisting of pembrolizumab, nivolumab, cemiplima, durvalumab, and atezolizumab.

Preferably, the chemotherapeutic agent is a platinum doublet.

For example, the immunotherapeutic agent is selected from the group consisting of pembrolizumab, nivolumab, cemiplima, durvalumab, and atezolizumab, and/or the chemotherapeutic agent is a platinum doublet.

As to other immunotherapeutic or chemotherapeutic agents it is referred to the first to eleventh aspect of the present invention.

The patient suffering from cancer is preferably a mammal, in particular a human.

In a further aspect, the present invention relates a method of treating a patient suffering from cancer with immunochemotherapy or immunotherapy comprising the steps of
(i) carrying out the method according to the twelfth aspect, thereby identifying the patient as patient who will (likely) benefit from immunochemotherapy or immunotherapy, and
(ii) treating the patient with said therapy.

As mentioned above, a patient having a (miRisk) score > about −0.073 will (likely) benefit from immunochemotherapy and is subsequently treated with said therapy. Alternatively, a patient having a (miRisk) score ≤ about −0.073 will (likely) benefit from immunochemotherapy or immunotherapy and is treated with one of said therapies. In this case, immunotherapy is usually chosen as it is of less therapeutic burden to the patient than immunotherapy in combination with chemotherapy.

Preferably, the immunotherapeutic agent is an inhibitor targeting PD-1/PD-L1. More preferably, the inhibitor targeting PD-1/PD-L1 is selected from the group consisting of pembrolizumab, nivolumab, cemiplima, durvalumab, and atezolizumab.

Preferably, the chemotherapeutic agent is a platinum doublet.

For example, the immunotherapeutic agent is selected from the group consisting of pembrolizumab, nivolumab, cemiplima, durvalumab, and atezolizumab, and/or the chemotherapeutic agent is a platinum doublet.

As to other immunotherapeutic or chemotherapeutic agents it is referred to the first to eleventh aspect of the present invention.

The patient suffering from cancer is preferably a mammal, in particular a human.

In a fourteenth aspect, the present invention relates to (an (in vitro) use of) a kit for selecting a patient suffering from cancer to (likely) benefit from immunochemotherapy or immunotherapy, wherein said kit comprises:
means for determining the level of at least one miRNA having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80%, preferably at least 85%, more preferably at least 90%, and even more preferably at least 95% or 99%, e.g. 80, 81, 82, 83, 84, 85, 86, 87, 88, 89, 90, 91, 92, 93, 94, 95, 96, 97, 98, or 99%, sequence identity thereto in a blood sample of a patient suffering from cancer, wherein the patient has optionally a PD-L1 Tumor Proportion Score (TPS) of ≥50%.

It is preferred that the patient having a PD-L1 TPS of ≥50% has no oncogene activating mutations. Such oncogene activating mutations may be EGFR mutations, BRAF V660e mutations, ALK translocations, and/or ROS-1 translocations.

It is further (alternatively or additionally) preferred that the cancer is eligible for immunochemotherapy or immunotherapy. A cancer eligible for immunochemotherapy is sensitive to an immunotherapeutic agent and a chemotherapeutic agent. A cancer eligible for immunotherapy is sensitive to an immunotherapeutic agent.

Specifically, the cancer eligible for immunotherapy is sensitive to immune checkpoint inhibition.

More specifically, the immune checkpoint inhibition is accompanied with the administration of at least one immune checkpoint inhibitor. Even more specifically, the at least one immune checkpoint inhibitor is an inhibitor targeting PD-1/PD-L1 or the PD-1/PD-L1 pathway.

It is also (alternatively or additionally) preferred that the cancer is selected from the group consisting of lung cancer, breast cancer, cervical cancer, gastric cancer, bladder cancer, skin cancer, nasopharyngeal cancer, neuroendrocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

It is more preferred that the cancer is selected from the group consisting of lung cancer, breast cancer, cervical cancer, gastric cancer, skin cancer, and bladder cancer. Said cancer is eligible for immunochemotherapy or immunotherapy.

It is even more preferred that the lung cancer is non-small-cell lung carcinoma (NSCLC).

It is still even more preferred that the non-small-cell lung carcinoma (NSCLC) is late stage NSCLC. It is most preferred that the late stage NSCLC is NSCLC of stage IV.

As mentioned above, the kit comprises means for determining the level of at least one miRNA having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80%, preferably at least 85%, more preferably at least 90%, and even more preferably at least 95% or 99%, e.g. 80, 81, 82, 83, 84, 85, 86, 87, 88, 89, 90, 91, 92, 93, 94, 95, 96, 97, 98, or 99%, sequence identity thereto in a blood sample of a patient suffering from cancer.

Specifically, the kit comprises
means for carrying out next generation sequencing (NGS),
at least one polynucleotide (probe) for detecting the at least one miRNA,
at least one primer (e.g. a primer pair) for binding the at least one miRNA, and/or
at least one antibody capable of binding a hybrid of said at least one polynucleotide (probe) and said at least one miRNA.

Said means allow to determine the level of the at least one miRNA in a blood sample of a patient suffering from cancer and, thus, to select a patient suffering from cancer to (likely) benefit from immunochemotherapy or immunotherapy.

The at least one polynucleotide (probe) may be part of a microarray/biochip or may be attached to beads of a beads-based multiplex system. The at least one polynucleotide (primer, primer pair) may be part of a RT-PCR system, a PCR-system, or a next generation sequencing system.

Said means may further comprise a microarray, a RT-PCT system, a PCR-system, a flow cytometer, a Luminex system, and/or a next generation sequencing system.

As mentioned above, the (level of the) at least one miRNA is detected/determined in a blood sample of the patient suffering from cancer. As to the preferred embodiments of the blood sample, it is referred to the first to eleventh aspect of the present invention.

More specifically, the kit comprises means for determining the level of PD-L1, in particular in a biological sample, such as a tissue sample, from a patient suffering from cancer. Said means can encompass a polynucleotide (probe), a primer (e.g. a primer pair), and/or an antibody being PD-L1 specific.

Even more specifically, the kit is useful for conducting the method of the twelfth aspect of the present invention.

The kit may comprise instructions on how to carry out the method according to the twelfth aspect of the present invention.

The kit may further comprise
a container, and/or
a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise the (miRisk) cut-off score. In case that the data carrier comprises an access-code which allows the access to a database, said (miRisk) cut-off score may be deposited in this database.

The data carrier may also comprise information or instructions on how to carry out the method according to the twelfth aspect of the present invention.

The kit may also comprise materials desirable from a commercial and user standpoint including a buffer(s), a reagent(s) and/or a diluent(s) for determining the level mentioned above.

The patient suffering from cancer is preferably a mammal, in particular a human.

In a fifteenth aspect, the present invention relates to an immunotherapeutic agent for use in the treatment of cancer in a patient, in combination with a chemotherapeutic agent,
wherein the patient has
a PD-L1 Tumor Proportion Score (TPS) of ≥50%, and
a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

In this respect, it should be noted that the miRNA having a nucleotide sequence according to SEQ ID NO: 1 is miR-2115-3p, the miRNA having a nucleotide sequence according to SEQ ID NO: 39 is miR-218-5p, the miRNA having a nucleotide sequence according to SEQ ID NO: 13 is miR-224-5p, the miRNA having a nucleotide sequence according to SEQ ID NO: 7 is miR-4676-3p, and the miRNA having a nucleotide sequence according to SEQ ID NO: 34 is miR-6503-5p.

In particular, the (miRisk) score is determined in a blood sample of the patient. As to the preferred blood sample, it is referred to the first to eleventh aspect of the present invention.

It is preferred that the patient having a PD-L1 TPS of ≥50% has no oncogene activating mutations. Such oncogene activating mutations may be EGFR mutations, BRAF V660e mutations, ALK translocations, and/or ROS-1 translocations.

It is further (alternatively or additionally) preferred that the cancer is eligible for immunochemotherapy or immunotherapy. A cancer eligible for immunochemotherapy is sensitive to an immunotherapeutic agent and a chemotherapeutic agent. A cancer eligible for immunotherapy is sensitive to an immunotherapeutic agent.

Specifically, the cancer eligible for immunotherapy is sensitive to immune checkpoint inhibition.

More specifically, the immune checkpoint inhibition is accompanied with the administration of at least one immune checkpoint inhibitor. Even more specifically, the at least one immune checkpoint inhibitor is an inhibitor targeting PD-1/PD-L1 or the PD-1/PD-L1 pathway.

It is also (alternatively or additionally) preferred that the cancer is selected from the group consisting of lung cancer, breast cancer, cervical cancer, gastric cancer, bladder cancer, skin cancer, nasopharyngeal cancer, neuroendrocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

It is more preferred that the cancer is selected from the group consisting of lung cancer, breast cancer, cervical cancer, gastric cancer, skin cancer, and bladder cancer. Said cancer is eligible for immunochemotherapy or immunotherapy.

It is even more preferred that the lung cancer is non-small-cell lung carcinoma (NSCLC).

It is still even more preferred that the non-small-cell lung carcinoma (NSCLC) is late stage NSCLC. It is most preferred that the late stage NSCLC is NSCLC of stage IV.

Specifically, the immunotherapeutic agent is an inhibitor targeting PD-1/PD-L1. More specifically, the inhibitor targeting PD-1/PD-L1 is selected from the group consisting of pembrolizumab, nivolumab, cemiplima, durvalumab, and atezolizumab.

Specifically, the chemotherapeutic agent used in combination is a platinum doublet.

For example, the immunotherapeutic agent is selected from the group consisting of pembrolizumab, nivolumab, cemiplima, durvalumab, and atezolizumab and the chemotherapeutic agent is a platinum doublet.

As to other immunotherapeutic or chemotherapeutic agents it is referred to the first to eleventh aspect of the present invention.

The components of the combination, i.e. the immunotherapeutic agent and the chemotherapeutic agent, may be administered together or independent from each other (e.g. one after the other).

The patient suffering from cancer is preferably a mammal, in particular a human.

This aspect of the present invention can also be worded as follows: In a fifteenth aspect, the present invention relates to the use of an immunotherapeutic agent for the manufacture of a medicament for the treatment of cancer, in combination with a chemotherapeutic agent,
wherein the patient has
a PD-L1 Tumor Proportion Score (TPS) of ≥50%, and
a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

Alternatively, the present invention relates in a fifteenth aspect to a method for treating cancer in a patient comprising the steps of:
(i) determining a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 )$$

in a blood sample of a patient, and
(ii) administering (an effective amount of) an immunotherapeutic agent in combination with a chemotherapeutic agent to the patient in need thereof,
wherein the patient has a PD-L1 Tumor Proportion Score (TPS) of ≥50%.

In a sixteenth aspect, the present invention relates to a chemotherapeutic agent for use in the treatment of cancer in a patient, in combination with an immunotherapeutic agent,
wherein the patient has
a PD-L1 Tumor Proportion Score (TPS) of ≥50%, and
a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

In this respect, it should be noted that the miRNA having a nucleotide sequence according to SEQ ID NO: 1 is miR-2115-3p, the miRNA having a nucleotide sequence according to SEQ ID NO: 39 is miR-218-5p, the miRNA having a nucleotide sequence according to SEQ ID NO: 13 is miR-224-5p, the miRNA having a nucleotide sequence according to SEQ ID NO: 7 is miR-4676-3p, and the miRNA having a nucleotide sequence according to SEQ ID NO: 34 is miR-6503-5p.

In particular, the (miRisk) score is determined in a blood sample of the patient. As to the preferred blood sample, it is referred to the first to eleventh aspect of the present invention.

It is preferred that the patient having a PD-L1 TPS of ≥50% has no oncogene activating mutations. Such oncogene activating mutations may be EGFR mutations, BRAF V660e mutations, ALK translocations, and/or ROS-1 translocations.

It is further (alternatively or additionally) preferred that the cancer is eligible for immunochemotherapy or immunotherapy. A cancer eligible for immunochemotherapy is sensitive to an immunotherapeutic agent and a chemotherapeutic agent. A cancer eligible for immunotherapy is sensitive to an immunotherapeutic agent.

Specifically, the cancer eligible for immunotherapy is sensitive to immune checkpoint inhibition.

More specifically, the immune checkpoint inhibition is accompanied with the administration of at least one immune checkpoint inhibitor. Even more specifically, the at least one immune checkpoint inhibitor is an inhibitor targeting PD-1/PD-L1 or the PD-1/PD-L1 pathway.

It is also (alternatively or additionally) preferred that the cancer is selected from the group consisting of lung cancer, breast cancer, cervical cancer, gastric cancer, bladder cancer, skin cancer, nasopharyngeal cancer, neuroendrocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

It is more preferred that the cancer is selected from the group consisting of lung cancer, breast cancer, cervical cancer, gastric cancer, skin cancer, and bladder cancer. Said cancer is eligible for immunochemotherapy or immunotherapy.

It is even more preferred that the lung cancer is non-small-cell lung carcinoma (NSCLC).

It is still even more preferred that the non-small-cell lung carcinoma (NSCLC) is late stage NSCLC. It is most preferred that the late stage NSCLC is NSCLC of stage IV.

Specifically, the chemotherapeutic agent is a platinum doublet.

Specifically, the immunotherapeutic agent used in combination is an inhibitor targeting PD-1/PD-L1. More specifically, the inhibitor targeting PD-1/PD-L1 is selected from the group consisting of pembrolizumab, nivolumab, cemiplima, durvalumab, and atezolizumab.

For example, the chemotherapeutic agent is a platinum doublet and the immunotherapeutic agent is selected from the group consisting of pembrolizumab, nivolumab, cemiplima, durvalumab, and atezolizumab.

As to other chemotherapeutic or immunotherapeutic agents it is referred to the first to eleventh aspect of the present invention.

The components of the combination, i.e. the chemotherapeutic agent and the immunotherapeutic agent, may be administered together or independent from each other (e.g. one after the other).

The patient suffering from cancer is preferably a mammal, in particular a human.

This aspect of the present invention can also be worded as follows: In a sixteenth aspect, the present invention relates to the use of a chemotherapeutic agent for the manufacture of a medicament for the treatment of cancer, in combination with an immunotherapeutic agent,
wherein the patient has
a PD-L1 Tumor Proportion Score (TPS) of ≥50%, and
a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

Alternatively, the present invention relates in a sixteenth aspect to a method for treating cancer in a patient comprising the steps of
(i) determining a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 )$$

in a blood sample of a patient, and
(ii) administering (an effective amount of) a chemotherapeutic agent in combination with an immunotherapeutic agent to the patient in need thereof,
wherein the patient has a PD-L1 Tumor Proportion Score (TPS) of ≥50%.

In a seventeenth aspect, the present invention relates to an immunotherapeutic agent for use in the treatment of cancer in a patient, optionally in combination with a chemotherapeutic agent,
wherein the patient has
a PD-L1 Tumor Proportion Score (TPS) of ≥50%, and
a (miRisk) score ≤ about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

In this respect, it should be noted that the miRNA having a nucleotide sequence according to SEQ ID NO: 1 is miR-2115-3p, the miRNA having a nucleotide sequence according to SEQ ID NO: 39 is miR-218-5p, the miRNA having a nucleotide sequence according to SEQ ID NO: 13 is miR-224-5p, the miRNA having a nucleotide sequence according to SEQ ID NO: 7 is miR-4676-3p, and the miRNA having a nucleotide sequence according to SEQ ID NO: 34 is miR-6503-5p.

In particular, the (miRisk) score is determined in a blood sample of the patient. As to the preferred blood sample, it is referred to the first to eleventh aspect of the present invention.

It is preferred that the patient having a PD-L1 TPS of ≥50% has no oncogene activating mutations. Such oncogene activating mutations may be EGFR mutations, BRAF V660e mutations, ALK translocations, and/or ROS-1 translocations.

It is further (alternatively or additionally) preferred that the cancer is eligible for immunochemotherapy or immunotherapy. A cancer eligible for immunochemotherapy is sensitive to an immunotherapeutic agent and a chemotherapeutic agent. A cancer eligible for immunotherapy is sensitive to an immunotherapeutic agent.

Specifically, the cancer eligible for immunotherapy is sensitive to immune checkpoint inhibition.

More specifically, the immune checkpoint inhibition is accompanied with the administration of at least one immune checkpoint inhibitor. Even more specifically, the at least one immune checkpoint inhibitor is an inhibitor targeting PD-1/PD-L1 or the PD-1/PD-L1 pathway.

It is also (alternatively or additionally) preferred that the cancer is selected from the group consisting of lung cancer, breast cancer, cervical cancer, gastric cancer, bladder cancer, skin cancer, nasopharyngeal cancer, neuroendrocrine cancer, colon cancer, urothelial cancer, liver cancer, ovarian cancer, esophageal cancer, pancreatic cancer, kidney cancer, stomach cancer, esophageal cancer, renal cancer, head and neck cancer, brain cancer, lymphatic cancer, blood cancer, squamous cell cancer, laryngeal cancer, retina cancer, prostate cancer, uterine cancer, testicular cancer, bone cancer, lymphoma, and leukemia.

It is more preferred that the cancer is selected from the group consisting of lung cancer, breast cancer, cervical cancer, gastric cancer, skin cancer, and bladder cancer. Said cancer is eligible for immunochemotherapy or immunotherapy.

It is even more preferred that the lung cancer is non-small-cell lung carcinoma (NSCLC).

It is still even more preferred that the non-small-cell lung carcinoma (NSCLC) is late stage NSCLC. It is most preferred that the late stage NSCLC is NSCLC of stage IV.

Specifically, the immunotherapeutic agent is an inhibitor targeting PD-1/PD-L1. More specifically, the inhibitor targeting PD-1/PD-L1 is selected from the group consisting of pembrolizumab, nivolumab, cemiplima, durvalumab, and atezolizumab.

Specifically, the chemotherapeutic agent which is optionally used in combination with an immunotherapeutic agent is a platinum doublet.

For example, the immunotherapeutic agent is selected from the group consisting of pembrolizumab, nivolumab, cemiplima, durvalumab, and atezolizumab and the chemotherapeutic agent is a platinum doublet.

As to other immunotherapeutic or chemotherapeutic agents it is referred to the first to eleventh aspect of the present invention.

The components of the combination, if present, i.e. the immunotherapeutic agent and the chemotherapeutic agent, may be administered together or independent from each other (e.g. one after the other).

The patient suffering from cancer is preferably a mammal, in particular a human.

This aspect of the present invention can also be worded as follows: In a seventeenth aspect, the present invention relates to the use of an immunotherapeutic agent for the manufacture of a medicament for the treatment of cancer in a patient, optionally in combination with a chemotherapeutic agent,
wherein the patient has
a PD-L1 Tumor Proportion Score (TPS) of ≥50%, and
a (miRisk) score ≤ about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

Alternatively, the present invention relates in a seventeenth aspect to a method for treating cancer in a patient comprising the steps of:
(i) determining a (miRisk) score ≤ about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 )$$

in a blood sample of a patient, and
(ii) administering (an effective amount of) an immunotherapeutic agent, optionally in combination with a chemotherapeutic agent, to the patient in need thereof,
wherein the patient has a PD-L1 Tumor Proportion Score (TPS) of ≥50%.

It is most preferred that the cancer in the twelfth to seventeenth aspect of the present invention is lung cancer.

In this respect, the present invention relates to
1. A method of selecting a patient suffering from lung cancer to benefit from immunochemotherapy or immunotherapy comprising the step of:
   determining the level of at least one miRNA in a blood sample of a patient suffering from lung cancer,
   wherein the at least one miRNA has a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto, and
   wherein the patient has a PD-L1 Tumor Proportion Score (TPS) of ≥50%.
2. The method of item 1, wherein the patient having a PD-L1 TPS of ≥50% has no oncogene activating mutations.
3. The method of items 1 or 2, wherein the lung cancer is non-small-cell lung carcinoma (NSCLC).
4. The method of item 3, wherein the non-small-cell lung carcinoma (NSCLC) is late stage NSCLC.
5. The method of item 4, wherein the late stage NSCLC is NSCLC of stage IV.
6. The method of any one of items 1 to 5, wherein the level of the at least one miRNA is compared to a (miRisk) cut-off score.
7. The method of item 6, wherein
   the level of the at least one miRNA above the (miRisk) cut-off score indicates that the patient will benefit from immunochemotherapy, or
   the level of the at least one miRNA below or equal to the (miRisk) cut-off score indicates that the patient will benefit from immunochemotherapy or immunotherapy.
8. The method of any one of items 1 to 7, wherein the method further comprises the step of determining a (miRisk) score by summarizing the weighted levels of at least two, at least three, at least four, or five miRNAs having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto.
9. The method of item 8, wherein the (miRisk) score is determined by summarizing the weighted levels of five miRNAs having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), and SEQ ID NO: 34 (miR-6503-5p).
10. The method of items 8 or 9, wherein the (miRisk) score is calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) ,$$

wherein RPM means reads per million.
11. The method of any one of items 8 to 10, wherein the (miRisk) score is compared to a (miRisk) cut-off score.
12. The method of item 11, wherein the (miRisk) cut-off score is about −0.073.
13. The method of item 12, wherein
   a (miRisk) score > about −0.073 indicates that the patient will benefit from immunochemotherapy, or
   a (miRisk) score ≤ about −0.073 indicates that the patient will benefit from immunochemotherapy or immunotherapy.
14. A method of determining whether to treat a patient suffering from lung cancer with immunochemotherapy or immunotherapy comprising the steps of
   (i) carrying out the method of any one of items 1 to 13, thereby identifying the patient as patient who will benefit from immunochemotherapy or immunotherapy, and
   (ii) assigning the patient to said therapy.
15. A method of treating a patient suffering from lung cancer with immunochemotherapy or immunotherapy comprising the steps of
   (i) carrying out the method of any one of items 1 to 13, thereby identifying the patient as patient who will benefit from immunochemotherapy or immunotherapy, and
   (ii) treating the patient with said therapy.
16. A kit for selecting a patient suffering from lung cancer to benefit from immunochemotherapy or immunotherapy, wherein said kit comprises:
   means for determining the level of at least one miRNA having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto in a blood sample of a patient suffering from lung cancer, wherein the patient has a PD-L1 Tumor Proportion Score (TPS) of ≥50%.
17. The kit of item 16, wherein the patient having a PD-L1 TPS of ≥50% has no oncogene activating mutations.
18. The kit of items 16 or 17, wherein the lung cancer is non-small-cell lung carcinoma (NSCLC).
19. The kit of item 18, wherein the non-small-cell lung carcinoma (NSCLC) is late stage NSCLC.
20. The kit of item 19, wherein the late stage NSCLC is NSCLC of stage IV.
21. The kit of any one of items 16 to 20, wherein said kit further comprises instructions on how to carry out the method of any one of items 1 to 13.
22. The kit of any one of items 16 to 21, wherein the kit is useful for conducting the method of any one of items 1 to 13.
23. An immunotherapeutic agent for use in the treatment of lung cancer in a patient, in combination with a chemotherapeutic agent,
   wherein the patient has
   a PD-L1 Tumor Proportion Score (TPS) of ≥50%, and
   a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

24. The immunotherapeutic agent for use of item 23, wherein the patient having a PD-L1 TPS of ≥50% has no oncogene activating mutations.
25. The immunotherapeutic agent for use of items 23 or 24, wherein the lung cancer is non-small-cell lung carcinoma (NSCLC).
26. The immunotherapeutic agent for use of item 25, wherein the non-small-cell lung carcinoma (NSCLC) is late stage NSCLC.
27. The immunotherapeutic agent for use of item 26, wherein the late stage NSCLC is NSCLC of stage IV.
28. The immunotherapeutic agent for use of any one of items 23 to 27, wherein the immunotherapeutic agent is an inhibitor targeting PD-1/PD-L1.
29. The immunotherapeutic agent for use of item 28, wherein the inhibitor targeting PD-1/PD-L1 is selected from the group consisting of pembrolizumab, nivolumab, cemiplima, durvalumab, and atezolizumab.
30. A chemotherapeutic agent for use in the treatment of lung cancer in a patient, in combination with an immunotherapeutic agent,
   wherein the patient has
   a PD-L1 Tumor Proportion Score (TPS) of ≥50%, and
   a (miRisk) score > about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

31. The chemotherapeutic agent for use of item 30, wherein the patient having a PD-L1 TPS of ≥50% has no oncogene activating mutations.
32. The chemotherapeutic agent for use of items 30 or 31, wherein the lung cancer is non-small-cell lung carcinoma (NSCLC).
33. The chemotherapeutic agent for use of item 32, wherein the non-small-cell lung carcinoma (NSCLC) is late stage NSCLC.
34. The chemotherapeutic agent for use of item 33, wherein the late stage NSCLC is NSCLC of stage IV.
35. The chemotherapeutic agent for use of any one of items 30 to 34, wherein the chemotherapeutic agent is platinum doublet.
36. An immunotherapeutic agent for use in the treatment of lung cancer in a patient, optionally in combination with a chemotherapeutic agent,
   wherein the patient has
   a PD-L1 Tumor Proportion Score (TPS) of ≥50%, and
   a (miRisk) score ≤ about −0.073 calculated according to the following formula:

$$( miRisk ) score = ( ln ( SEQ ID NO : 1 ( miR ‐ 2115 ‐ 3 p ) RPM + 1 ) \times 2.190467 ) + ( ln ( SEQ ID NO : 39 ( miR ‐ 218 ‐ 5 p ) RPM + 1 ) \times 0.303095 ) + ( ln ( SEQ ID NO : 13 ( miR ‐ 224 ‐ 5 p ) RPM + 1 ) \times 0.598415 ) + ( ln ( SEQ ID NO : 7 ( miR ‐ 4676 ‐ 3 p ) RPM + 1 ) \times 1.101122 ) + ( ln ( SEQ ID NO : 34 ( miR ‐ 6503 ‐ 5 p ) RPM + 1 ) \times 0.958823 ) .$$

37. The immunotherapeutic agent for use of item 36, wherein the patient having a PD-L1 TPS of ≥50% has no oncogene activating mutations.
38. The immunotherapeutic agent for use of items 36 or 37, wherein the lung cancer is non-small-cell lung carcinoma (NSCLC).
39. The immunotherapeutic agent for use of item 38, wherein the non-small-cell lung carcinoma (NSCLC) is late stage NSCLC.
40. The immunotherapeutic agent for use of item 39, wherein the late stage NSCLC is NSCLC of stage IV.
41. The immunotherapeutic agent for use of any one of items 36 to 40, wherein the immunotherapeutic agent is an inhibitor targeting PD-1/PD-L1.
42. The immunotherapeutic agent for use of item 41, wherein the inhibitor targeting PD-1/PD-L1 is selected from the group consisting of pembrolizumab, nivolumab, cemiplima, durvalumab and atezolizumab.
43. The immunotherapeutic agent for use of any one of items 36 to 42, wherein the optional chemotherapeutic agent is platinum doublet.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIGS. 1A-1C: (A) Unbiased pipeline for univariate model selection. 1) The data is split 50:50 into train and test sets. 2) Univariate Cox proportional hazards models are fit to each feature within the test set. 3) The single best model is used to make predictions on the unseen test data. 4) The predictions are fit to a Cox proportional hazards model and whose significance is a marker of the accuracy of the predictions. This process can be repeated iteratively with random 50:50 splits to generate a distribution of model performance. (B) The distribution of model performance using the univariate pipeline as applied to correctly annotated survival data. All models to the left of the red vertical line have made statistically significant predictions on test data. (C) Four iterations of the pipeline on data with randomized survival information. Few significantly predictive models are generated.

FIGS. 2A-2D: (A) Unbiased pipeline for multivariate model selection. 1) The data is split 50:50 into train and test sets. 2) Multivariate Cox proportional hazards models are fit to each feature within the test set and all models with a significance below 0.001 are selected to create a multivariate model. 3) The multivariate model is used to make predictions on the unseen test data. 4) The predictions are fit to a Cox proportional hazards model and whose significance is a marker of the accuracy of the predictions. This process can be repeated iteratively with random 50:50 splits to generate a distribution of model performance. (B) The distribution of model performance using the multivariate pipeline as applied to correctly annotated survival data. All models to the left of the red vertical line have made statistically significant predictions on test data. A summary of the best models can be found in Table 2. (C) The best model (miR-2115-3p and miR-25-3p) was fit to a representative 50% split test data and used to classify patients as high or low risk by a cutoff of median risk score. (D) The previously fit model was used to make predictions on the unseen test data and classified into high and low risk using the previously defined risk threshold. Survival is significantly longer in the low-risk group (log-rank test p-value 0.011).

FIGS. 3A-3C: (A) miR-2115-3p shows a strikingly cell type specific expression pattern, with expression restricted to cells of a myeloid lineage. (B) PD-L1 (CD274) contains a strong predicted miR-2115-3p target site within its 3′ UTR. (C) miR-2115-3p is downregulated in responders compared to non-responders.

FIGS. 4A-4F: Overall survival (OS) of NSCLC patients stratified by miRisk score. a-c Comparison of OS between low/high miRisk score groups in the Training Cohort (n=96), Validation Immunotherapy Cohort (n=84), and the Validation Immunochemotherapy Cohort (n=139). Significant differences in OS are observed in the Training and Validation Immunotherapy Cohorts but not the Validation Chemoimmunotherapy Cohort. d-e Comparison of OS between patients stratified by PD-L1 TPS in the Training Cohort (n=96), Validation Immunotherapy Cohort (n=84), and the Validation Immunochemotherapy Cohort (n=139). The differences in OS do not reach significance in any cohort. Hazard ratios (HR) and 95% confidence intervals were calculated using a univariable Cox regression analysis; P-values were calculated using the log-rank test. All statistical analyses were two-sided.

FIGS. 5A-5B: Overall survival (OS) of low and high-risk patients stratified by Random Forest model. a-b Comparison of OS between low-risk and high-risk patients in the Training Cohort (n=96), and Validation Immunotherapy Cohort (n=84).

FIGS. 6A-6J: miRisk miRNA expression in low-risk and high-risk patients. a-e Relative expression levels of the 5 miRisk miRNAs between low-risk and high-risk patients measured by small RNA sequencing. f-j Relative expression levels of the 5 miRisk miRNAs between low-risk and high-risk patients measured by qRT-PCR. The mean expression of triplicate measurements is shown. qRT-PCR expression was normalized according to the ΔCt method (Ct_{miRNA of interest}−Ct_{mean of HK miRNAs}). All statistical tests were two-tailed unpaired t tests. Error bars denote standard deviation. RPM=reads per million. *=P-value <0.05, **=P-value <0.005, ***=P-value <0.0005, ****=P-value <0.00005, ns=not significant.

FIG. 7: Overall survival of low and high miRisk patients in an External Validation Control Cohort. 19 stage IV NSCLC patients undergoing no treatment, recruited in Grosshansdorf, Germany were stratified by low/high miRisk score. No significant difference in OS survival curves is observed.

FIG. 8: Overall survival of low and high-risk patients stratified with a multivariable Cox Proportional Hazards model incorporating PD-L1 TPS and the miRisk miRNAs. a-c Comparison of OS between low/high groups in the Training Cohort (n=96), Validation Immunotherapy Cohort (n=84), and the Validation Immunochemotherapy Cohort (n=139). As with the miRisk model, significant differences in OS are observed in the Training and Validation Immunotherapy Cohorts but not the Validation Chemoimmunotherapy Cohort. The magnitude of the hazard ratios is less than observed in the miRisk model.

FIG. 9: Shows preferred miRNAs and miRNA combinations. The preferred miRNAs have a nucleotide sequence according to SEQ ID NO: 1, SEQ ID NO: 7, SEQ ID NO: 13, SEQ ID NO: 34, or SEQ ID NO: 39. In all aspects described herein, the combination of the miRNAs having a nucleotide sequence according to SEQ ID NO: 1, SEQ ID NO: 7, SEQ ID NO: 13, SEQ ID NO: 34, and SEQ ID NO: 39 is most preferred.

FIGS. 10A-10D: Overall survival of NSCLC patients stratified by miRisk. a-b Comparison of OS between low/high miRisk score groups in independent PD-1/PD-L1 inhibitor monotherapy validation (n=56), and the chemoimmunotherapy control cohorts (n=31). Significant differences in OS are observed in the independent validation cohort but not the control cohort. c-d Comparison of OS between immunomonotherapy (10) and immunochemotherapy (ICT) in miRisk stratified cohorts. Hazard ratios (HR) and 95% confidence intervals were calculated using a univariable Cox regression analysis; P-values were calculated using the log-rank test. All statistical analyses were two-sided.

### EXAMPLES

The examples given below are for illustrative purposes only and do not limit the invention described above in any way.

### 1. Example 1

97 PAX-gene samples from treatment naïve stage IV lung cancer patients were collected before they have been commenced on immunotherapy (with Pembrolizumab or Nivolumab), as well as corresponding clinical data including response and survival information. Small RNAs were extracted and sequenced in order to measure the miRNA expression profile and subsequently use it to develop predictive signatures.

Initial exploratory analysis was performed on the miRNA expression data in order to identify any miRNAs that may be predictive of survival. Univariate Cox proportional Hazards models were fit to each feature in turn to generate a short-list of miRNAs that were significantly associated with survival time (pval 0.05). This analysis resulted in 44 miRNAs that were predictive of survival and predictors of response to immunotherapy (Table 1).

TABLE 1
All features to which a significant (p value < 0.05)
univariate Cox proportional hazards model can be fit.

| SEQ ID NO: | miRNA | pval | direction | Hazard ratio |
|---|---|---|---|---|
| 1 | hsa-miR-2115-3p | 0.000002 | Down | 6.286393 |
| 2 | hsa-miR-25-3p | 0.003749 | Down | 4.839328 |
| 3 | hsa-miR-338-3p | 0.022522 | Down | 1.795678 |
| 4 | hsa-miR-199b-5p | 0.001832 | Down | 1.858293 |
| 5 | hsa-miR-643 | 0.006033 | Down | 2.721444 |
| 6 | hsa-miR-191-3p | 0.024509 | Down | 3.317036 |
| 7 | hsa-miR-4676-3p | 0.005820 | Down | 3.44821 |
| 8 | hsa-miR-145-3p | 0.005476 | Down | 2.154442 |
| 9 | hsa-miR-9-3p | 0.022052 | Down | 1.951624 |
| 10 | hsa-miR-23a-3p | 0.023158 | Down | 2.808446 |
| 11 | hsa-miR-194-5p | 0.048985 | Down | 2.489514 |
| 12 | hsa-miR-582-3p | 0.004656 | Down | 2.034688 |
| 13 | hsa-miR-224-5p | 0.049680 | Down | 1.437725 |
| 14 | hsa-miR-23b-3p | 0.032732 | Down | 2.167218 |
| 15 | hsa-miR-628-5p | 0.028995 | Down | 2.185384 |
| 16 | hsa-miR-223-3p | 0.020920 | Down | 3.058213 |
| 17 | hsa-miR-4454 | 0.039089 | Down | 1.665509 |
| 18 | hsa-miR-3688-3p | 0.035477 | Down | 2.402228 |
| 19 | hsa-miR-10399-5p | 0.011425 | Down | 2.427667 |
| 20 | hsa-miR-128-3p | 0.040368 | Down | 2.492601 |
| 21 | hsa-miR-27a-3p | 0.038876 | Down | 1.758165 |
| 22 | hsa-miR-27b-3p | 0.039738 | Down | 1.886114 |
| 23 | hsa-miR-145-5p | 0.009604 | Down | 2.194916 |
| 24 | hsa-miR-150-5p | 0.037651 | Up | 0.637021 |
| 25 | hsa-miR-11400 | 0.027225 | Down | 1.844059 |
| 26 | hsa-miR-330-5p | 0.019006 | Down | 3.058271 |
| 27 | hsa-miR-6764-3p | 0.039503 | Up | 0.429466 |
| 28 | hsa-miR-4772-3p | 0.018089 | Down | 1.701595 |
| 29 | hsa-miR-9-5p | 0.025060 | Down | 1.70617 |
| 30 | hsa-miR-769-5p | 0.011920 | Down | 2.242065 |
| 31 | hsa-miR-2115-5p | 0.006594 | Down | 2.718221 |
| 32 | hsa-miR-30e-3p | 0.006608 | Down | 3.200051 |
| 33 | hsa-miR-3688-5p | 0.033523 | Down | 2.226333 |
| 34 | hsa-miR-6503-5p | 0.033815 | Down | 1.576189 |
| 35 | hsa-miR-4662a-5p | 0.028520 | Down | 1.812149 |
| 36 | hsa-miR-3127-5p | 0.028417 | Up | 0.38666 |
| 37 | hsa-miR-6505-5p | 0.016464 | Down | 3.770965 |
| 38 | hsa-miR-150-3p | 0.042787 | Up | 0.639251 |
| 39 | hsa-miR-218-5p | 0.012072 | Down | 1.330489 |
| 40 | hsa-miR-1271-5p | 0.030997 | Down | 2.128774 |
| 41 | hsa-miR-450a-5p | 0.026432 | Down | 1.804774 |
| 42 | hsa-miR-551a | 0.021056 | Down | 1.898281 |
| 43 | hsa-miR-6826-3p | 0.020253 | Up | 0.63542 |
| 44 | hsa-miR-4690-3p | 0.032662 | Down | 2.496545 |

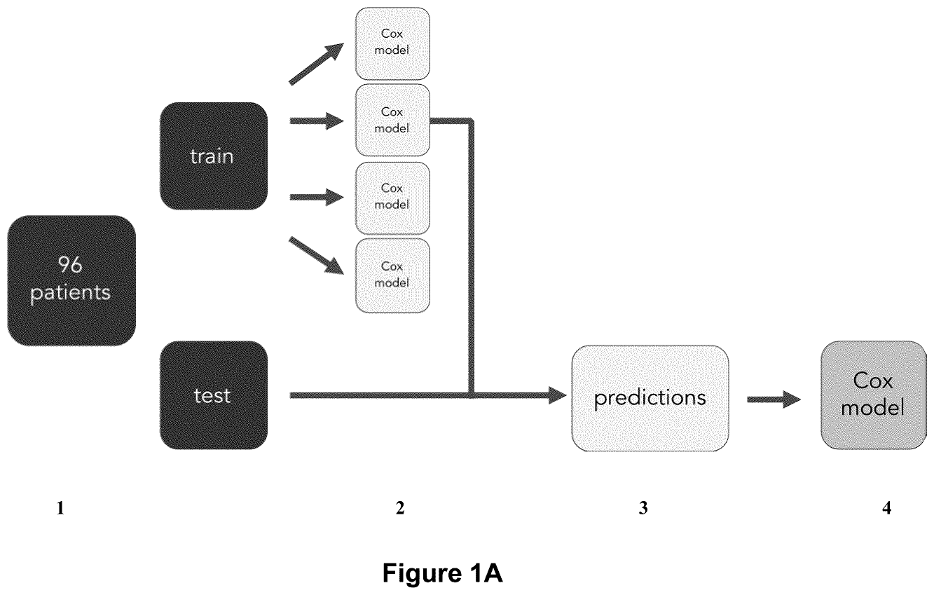

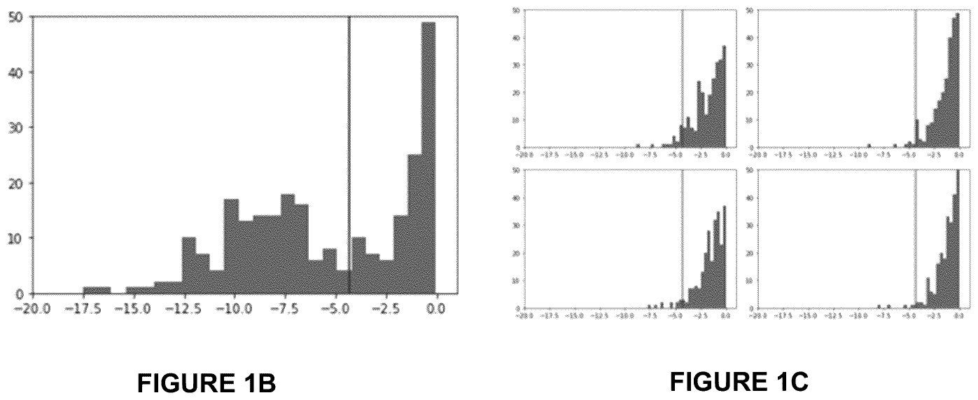

Next, an unbiased pipeline was built (FIG. 1A). This first splits the data into 50:50 train and test datasets. A univariate survival model (Cox proportional hazards model) is fit to each miRNA in turn within the training set and the best performing model is selected to make predictions on the unseen train dataset. The predictions on the train dataset are used as input to fit a further survival model. The predictive performance is Judged on the significance with which the predictions can be fit to the test dataset. This process is repeated iteratively 250 times in order to measure the distribution of model performance over a range of random test/train splits (FIG. 1B). A random permutation control was performed in which the pipeline was applied to miRNA expression data and randomized survival data (FIG. 1C). In 5400 of iterations on the true dataset, the pipeline selects miR-2115S-3p (SEQ ID NO: 1) as the most predictive feature (the most frequently selected feature in the randomized dataset is selected in only 8% of iterations). Together, this is evidence that miR-2115S-3p is associated with survival following immunotherapy treatment and that the strength of association is in excess of that observed with models fit to noise in the randomized datasets. The other 43 miRNAs that were predictive of survival and predictors of response to immunotherapy are listed in Table 1.

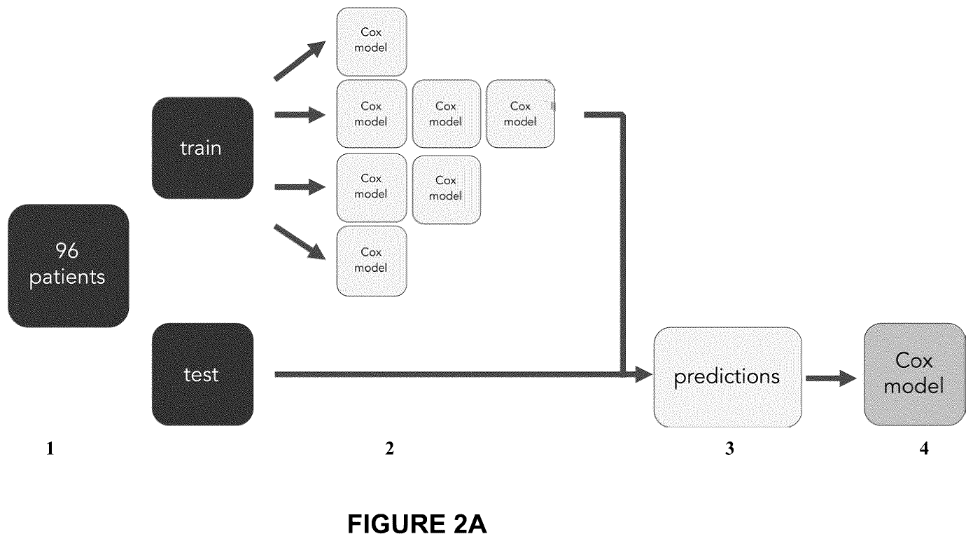

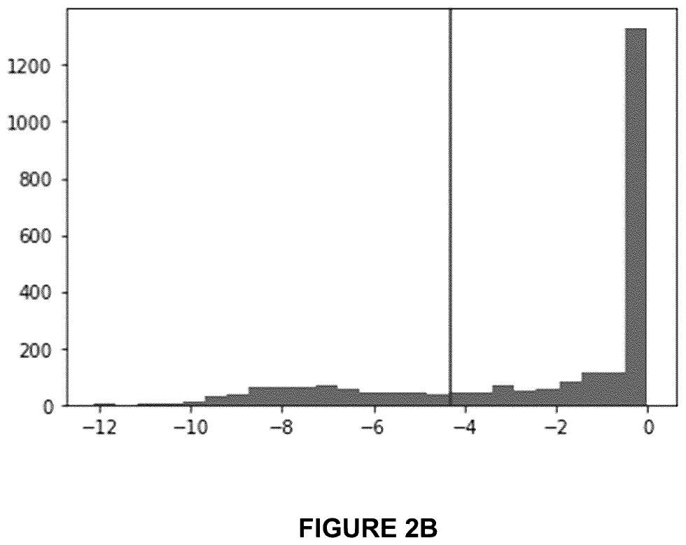

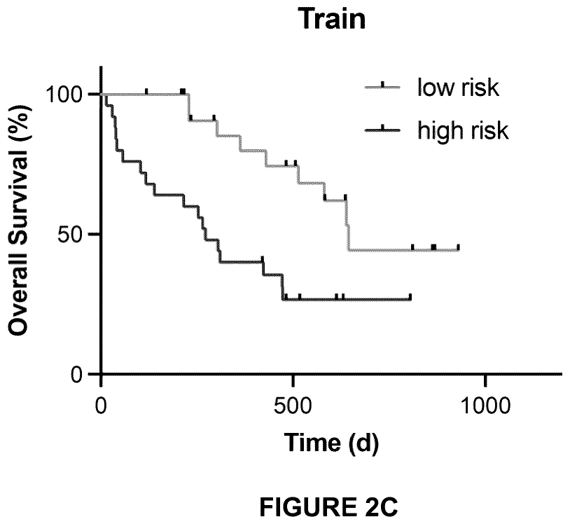

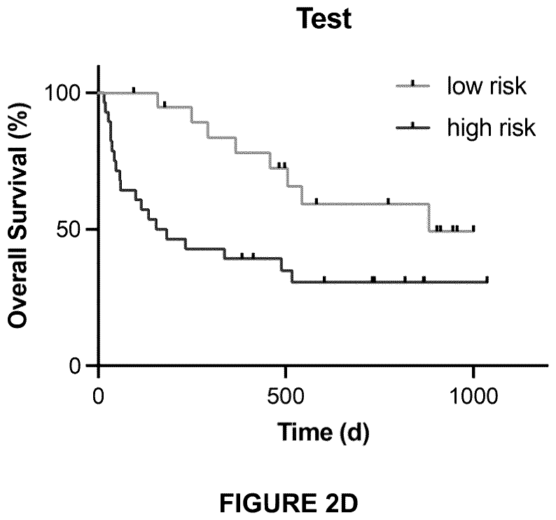

Finally, a second pipeline (FIG. 2A) was constructed. Data was split 50:50 into train and test datasets. Feature selection was performed with the test dataset by choosing all features to which a significant univariate Cox model could be fit (p value < threshold). The selected features were then used to fit a multivariate Cox model to the training data and make predictions on the test data. The predictions were fit to a univariate Cox model whose significance was a measure of predictive performance. This process was run 3500 times in order to identify optimal feature(s) and evaluate the model (FIG. 2B and Table 2). Preferred miRNAs and miRNA combinations (sets/signatures) are listed in Table 2.

TABLE 2
The feature or feature combinations selected and the significance
to which they make predictions on the test dataset from 3500
iterations of the multivariate pipeline (FIG. 2a).

| mIR | median_pval |
|---|---|
| [‘hsa-miR-25-3p’, ‘hsa-miR-2115-3p’] | 0.002911355 |
| [‘hsa-miR-2115-3p’] | 0.004042011 |
| [‘hsa-miR-2115-3p’, ‘hsa-miR-145-3p’] | 0.025733041 |
| [‘hsa-miR-191-3p’, ‘hsa-miR-2115-3p’] | 0.027674616 |
| [‘hsa-miR-199b-5p’, ‘hsa-miR-2115-3p’, | 0.028748348 |
| ‘hsa-miR-145-3p’] | |
| [‘hsa-miR-199b-5p’, ‘hsa-miR-2115-3p’] | 0.033263039 |
| [‘hsa-miR-643’, ‘hsa-miR-2115-3p’] | 0.038320555 |
| [‘hsa-miR-338-3p’, ‘hsa-miR-2115-3p’] | 0.041565352 |
| [‘hsa-miR-4676-3p’, ‘hsa-miR-2115-3p’] | 0.046961095 |
| [‘hsa-miR-582-3p’, ‘hsa-miR-2115-3p’] | 0.050758831 |
| [‘hsa-miR-338-3p’, ‘hsa-miR-199b-5p’, | 0.056894736 |
| ‘hsa-miR-2115-3p’] | |
| [‘hsa-miR-145-5p’, ‘hsa-miR-2115-3p’] | 0.065439475 |
| [‘hsa-miR-4772-3p’, ‘hsa-miR-2115-3p’] | 0.067901752 |
| [‘hsa-miR-2115-5p’, ‘hsa-miR-2115-3p’] | 0.072238243 |
| [‘hsa-miR-2115-3p’, ‘hsa-miR-6505-5p’] | 0.089982453 |
| [‘hsa-miR-2115-3p’, ‘hsa-miR-4690-3p’] | 0.092230639 |
| [‘hsa-miR-9-3p’, ‘hsa-miR-2115-3p’] | 0.106559183 |
| [‘hsa-miR-628-5p’, ‘hsa-miR-2115-3p’] | 0.107851422 |
| [‘hsa-miR-769-5p’, ‘hsa-miR-2115-3p’] | 0.109672403 |
| [‘hsa-miR-23a-3p’, ‘hsa-miR-2115-3p’] | 0.114178415 |
| [‘hsa-miR-10399-5p’, ‘hsa-miR-2115-3p’] | 0.119172603 |
| [‘hsa-miR-142-5p’, ‘hsa-miR-2115-3p’] | 0.131781996 |
| [‘hsa-miR-769-5p’, ‘hsa-miR-2115-5p’, | 0.137546967 |
| ‘hsa-miR-2115-3p’] | |
| [‘hsa-miR-145-5p’, ‘hsa-miR-2115-5p’, | 0.15545503 |
| ‘hsa-miR-2115-3p’] | |
| [‘hsa-miR-2115-3p’, ‘hsa-miR-6826-3p’] | 0.164364996 |

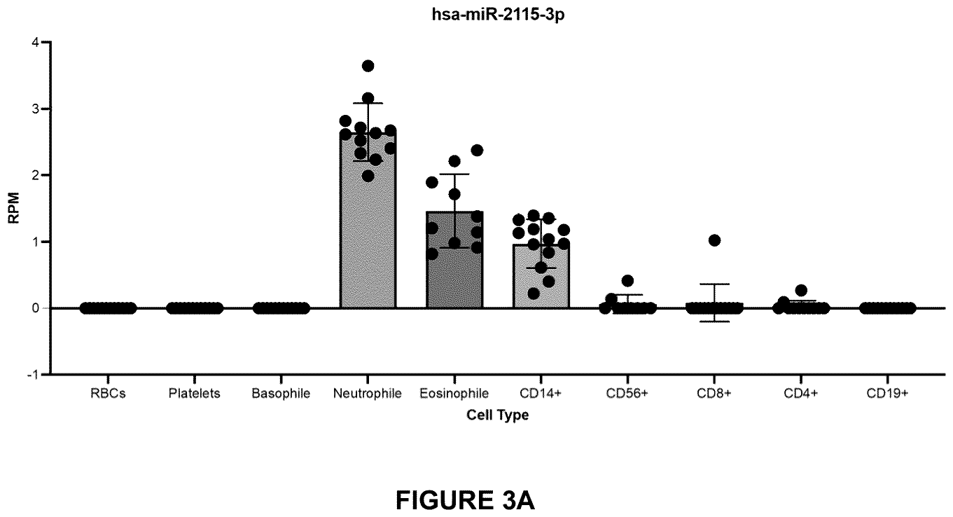

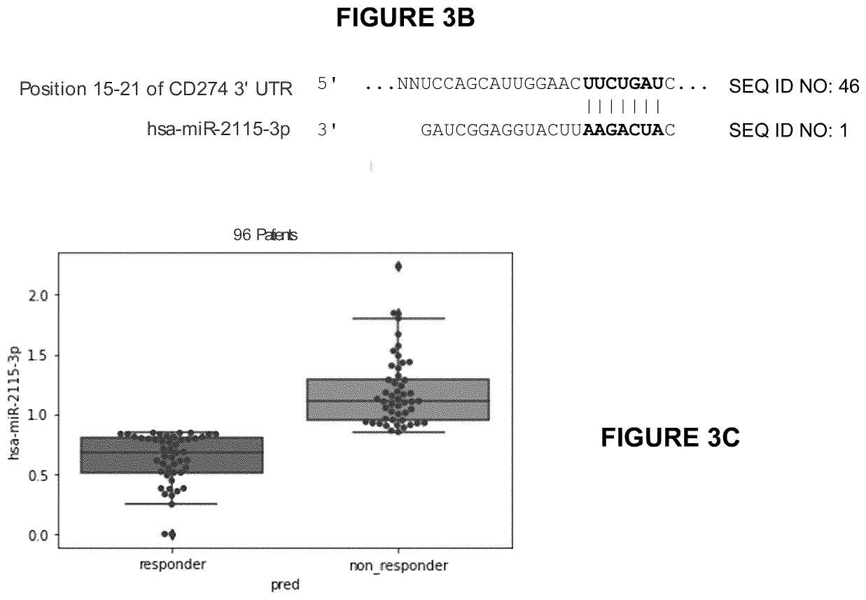

This optimized model (consisting of miR-2115-3p and miR-25-3p) was used to predict responder/non-responder status of patients within the training and test data sets (based on a threshold risk score defined as the median risk within the training data set). In both training and test datasets (single representative 50:50 split), it was possible to predict a cohort of responder patients with significantly increased survival (FIG. 2D). It was identified that miR-2115-3p is downregulated in responders compared to non-responders (FIG. 3C).

The miR-2115-3p stood out as the most predictive feature and was found to have specific expression in relevant cell populations within the blood (Monocytes and Neutrophils, FIG. 3A). Furthermore, a miR-2115-3p target site exists within the 3′ UTR of PD-L1 (FIG. 3B).

In summary: New miRNAs that are associated with response to immune checkpoint inhibitors of the PD-1/PD-L1 inhibitor class in treatment naïve stage IV NSCLC patients were identified. In addition, miRNA-based signatures that predict response to PD-1/PD-L1 inhibitors in stage IV NSCLC were revealed. Said miRNAs/miRNA-based signatures also allow survival prediction.

In Table 3, the identified miRNAs, with SEQ ID NOs and nucleotide sequences are listed:

TABLE 3
Identified miRNAs, SEQ ID NOs and corresponding
nucleotide sequences

| SEQ | | |
|---|---|---|
| ID | | |
| NO: | miRBase ID | Mature Sequence |
| 1 | hsa-miR-2115-3p | CAUCAGAAUUCAUGGAGGCUAG |
| 2 | hsa-miR-25-3p | CAUUGCACUUGUCUCGGUCUGA |
| 3 | hsa-miR-338-3p | UCCAGCAUCAGUGAUUUUGUUG |
| 4 | hsa-miR-199b-5p | CCCAGUGUUUAGACUAUCUGUUC |
| 5 | hsa-miR-643 | ACUUGUAUGCUAGCUCAGGUAG |
| 6 | hsa-miR-191-3p | GCUGCGCUUGGAUUUCGUCCCC |
| 7 | hsa-miR-4676-3p | CACUGUUUCACCACUGGCUCUU |
| 8 | hsa-miR-145-3p | GGAUUCCUGGAAAUACUGUUCU |
| 9 | hsa-miR-9-3p | AUAAAGCUAGAUAACCGAAAGU |
| 10 | hsa-miR-23a-3p | AUCACAUUGCCAGGGAUUUCC |
| 11 | hsa-miR-194-5p | UGUAACAGCAACUCCAUGUGGA |
| 12 | hsa-miR-582-3p | UAACUGGUUGAACAACUGAACC |
| 13 | hsa-miR-224-5p | UCAAGUCACUAGUGGUUCCGUUUAG |
| 14 | hsa-miR-23b-3p | AUCACAUUGCCAGGGAUUACCAC |
| 15 | hsa-miR-628-5p | AUGCUGACAUAUUUACUAGAGG |
| 16 | hsa-miR-223-3p | UGUCAGUUUGUCAAAUACCCCA |
| 17 | hsa-miR-4454 | GGAUCCGAGUCACGGCACCA |
| 18 | hsa-miR-3688-3p | UAUGGAAAGACUUUGCCACUCU |
| 19 | hsa-miR-10399-5p | AAUUACAGAUUGUCUCAGAGA |
| 20 | hsa-miR-128-3p | UCACAGUGAACCGGUCUCUUU |
| 21 | hsa-miR-27a-3p | UUCACAGUGGCUAAGUUCCGC |
| 22 | hsa-miR-27b-3p | UUCACAGUGGCUAAGUUCUGC |
| 23 | hsa-miR-145-5p | GUCCAGUUUUCCCAGGAAUCCCU |
| 24 | hsa-miR-150-5p | UCUCCCAACCCUUGUACCAGUG |
| 25 | hsa-miR-11400 | UCGGCUGUGUAUCUCUGUGUC |
| 26 | hsa-miR-330-5p | UCUCUGGGCCUGUGUCUUAGGC |
| 27 | hsa-miR-6764-3p | UCUCUGGUCUUUCCUUGACAG |
| 28 | hsa-miR-4772-3p | CCUGCAACUUUGCCUGAUCAGA |
| 29 | hsa-miR-9-5p | UCUUUGGUUAUCUAGCUGUAUGA |
| 30 | hsa-miR-769-5p | UGAGACCUCUGGGUUCUGAGCU |
| 31 | hsa-miR-2115-5p | AGCUUCCAUGACUCCUGAUGGA |
| 32 | hsa-miR-30e-3p | CUUUCAGUCGGAUGUUUACAGC |
| 33 | hsa-miR-3688-5p | AGUGGCAAAGUCUUUCCAUAU |
| 34 | hsa-miR-6503-5p | AGGUCUGCAUUCAAAUCCCCAGA |
| 35 | hsa-miR-4662a-5p | UUAGCCAAUUGUCCAUCUUUAG |
| 36 | hsa-miR-3127-5p | AUCAGGGCUUGUGGAAUGGGAAG |
| 37 | hsa-miR-6505-5p | UUGGAAUAGGGGAUAUCUCAGC |
| 38 | hsa-miR-150-3p | CUGGUACAGGCCUGGGGGACAG |
| 39 | hsa-miR-218-5p | UUGUGCUUGAUCUAACCAUGU |
| 40 | hsa-miR-1271-5p | CUUGGCACCUAGCAAGCACUCA |
| 41 | hsa-miR-450a-5p | UUUUGCGAUGUGUUCCUAAUAU |
| 42 | hsa-miR-551a | GCGACCCACUCUUGGUUUCCA |
| 43 | hsa-miR-6826-3p | CUCCCCUCUCUUUCCUGUUCAG |
| 44 | hsa-miR-4690-3p | GCAGCCCAGCUGAGGCCUCUG |

### 2. Example 2

### 2.1 Methods

### Patient Enrolment

This study was approved by the Heidelberg University (5-296/2016, 5-089/2019) and Grosshansdorf hospital ethics committee (AZ 12-238) and involved all patients with advanced NSCLC treated with immunotherapy alone or immunotherapy in combination with chemotherapy in the Thoraxklinik Heidelberg, for which blood samples were available. These were collected prospectively as published (Wessels et al., 2020) and provided by the Lungenbiobank Heidelberg for the present analysis according to the pertinent regulations. All eligible patients receiving immunotherapy alone were recruited to the Training Cohort. Eligible patients were included in the Immunotherapy or Immunochemotherapy Validation Cohorts depending on their respective treatments. Diagnosis of NSCLC was performed in the Institute of Pathology Heidelberg using tissue specimens according to the criteria of the current WHO classification (2015) for lung cancer. Clinical data and laboratory results were collected by a systematic review of patient records. The following clinical data were extracted: demographic, baseline clinical and tumor characteristics, including ECOG performance status (PS), smoking status, PD-L1 tumor proportion score (TPS), laboratory results, systemic and local anticancer treatments, date of progression, date of the last follow-up, and date of death. The neutrophil-to-lymphocyte ratio (NLR) was dichotomized at the bibliographical cut-off of 5, which corresponds to the median value for untreated patients (Suh, K. J. et al. (2018) Post-treatment neutrophil-to-lymphocyte ratio at week 6 is prognostic in patients with advanced non-small cell lung cancers treated with anti-PD-1 antibody. Cancer Immunol Immunother 67, 459-470; Valero, C. et al. (2021) Pretreatment neutrophil-to-lymphocyte ratio and mutational burden as biomarkers of tumor response to immune checkpoint inhibitors. Nat Commun 12, 729). PD-L1 TPS was assessed using the clone SP263 (Ventana/Roche, Mannheim, Germany) and trichotomized for analysis as <1, 1-49, and ≥50%, reflecting the relevant clinical thresholds in current treatment guidelines (Hanna, N. H. et al. (2020) Therapy for Stage IV Non-Small-Cell Lung Cancer Without Driver Alterations: ASCO and OH (CCO) Joint Guideline Update. J Clin Oncol 38, 1608-1632). In addition, a smaller cohort of patients with newly-diagnosed metastatic NSCLC opting for best supportive care was recruited from the Lungenclinic Grosshansdorf after approval of the local ethic committee (AZ 12-238) as a non-immunotherapy control.

### PAXgene Collection and Processing

PAXgene (PreAnalytiX, Hombrechtikon, Switzerland) blood samples were acquired as per manufacturer instructions, inverted immediately 10× and frozen at −20° C. within 2 hours. For long term storage the tubes were transferred to −80° C. Training Cohort. The PAXgene tubes were thawed overnight and extracted using the PAXgene Blood miRNA Kit (Qiagen, Venlo, Netherlands) as per manufacturer instructions. Eluates of 80 μl in Rnase-free water were stored in −80° C. Validation Cohorts. The PAXgene tubes were thawed overnight, and RNA was extracted using PAXgene Blood RNA Kit (Qiagen, Venlo, Netherlands). The flow through of the first column (RNA shredder column) was saved and stored in −20° C. and used for further extraction of RNAs<200 nts of length. For this purpose, the alcohol content of the sample was brought to 50% (w/v) with isopropanol and the samples were processed further using PAXgene Blood miRNA Kit, starting from the RNA column step. The RNA was eluted in 40 μl Rnase-free water and stored at −80° C.

### Small RNA-seq

100 ng of total RNA, or 5 μl of total RNA (for samples with concentration lower than 20 ng/μl) were used for QIAseq® miRNA Library Kit (Qiagen, Venlo, Netherlands), with blocking of previously reported highly abundant miRNAs (miR-16-5p, miR-486-5p, miR-451a (Roberts B S, Hardigan A A, Kirby M K, et al. Blocking of targeted microRNAs from next-generation sequencing libraries. Nucleic Acids Res. 2015; 43(21):e145. doi:10.1093/nar/gkv724, Juzenas S, Lindqvist C M, Ito G, et al. Depletion of erythropoietic miR-486-5p and miR-451a improves detectability of rare microRNAs in peripheral blood-derived small RNA sequencing libraries. NAR Genom Bioinform. 2020; 2 (1):lqaa008. Published 2020 Feb. 12. doi:10.1093/nargab/lqaa008)). cDNA was amplified using primers (data not shown) allowing unique dual indexing of the libraries and PCR products were cleaned-up using Mag-Bind TotalPure NGS beads (Omega Bio-Tek, Norcross, USA). PCR products following 18 cycles of amplification were assessed for size and uniformity using the Quantitative DNA kit on Fragment Analyzer (Agilent, Santa Clara, USA). The concentration of the PCR products was determined by Quant-iT dsDNA Assay-Kit (ThermoFisher Scientific, Waltham, USA), and equimolar library pools with up to 96 samples were prepared and sequenced on NextSeq 500 (Illumina, San Diego, California) with a 2.7 pM final pooled library concentration. A custom index 2 sequencing primer with the sequence 5′-GATCGTCGGACTGTAGAACTCTGAACGTGT-3′ (SEQ ID NO: 45) was used.

### Statistical Analysis

Non-penalized, univariable and multiviable Cox models for survival regression were fit in Python using the packages scikit-survival (version 0.15.1, (Pölsterl, S. (2020) scikit-survival: A Library for Time-to-Event Analysis Built on Top of scikit-learn. Journal of Machine Learning Research 21, 1-6) and Lifelines (version 0.26.0, (Davidson-Pilon, C. et al. (2021) CamDavidsonPilon/lifelines: 0.26.0) using the CoxPHSurvivalAnalysis and CoxPHFitter classes respectively with default parameter settings. In order to overcome the p>>n problem while modelling survival in high dimensional data, Witten and Tibshirani have proposed a number of methods including discrete feature selection using univariate and stepwise selection (Witten, D. M., and Tibshirani, R. (2010) Survival analysis with high-dimensional covariates. Stat Methods Med Res 19, 29-51). These have been implemented with the package scikit-learn (version 0.24.2, (Pedregosa, F., et al. (2012). Scikit-learn: Machine Learning in Python. Arxiv)) and the SelectFpr (selecting miRNA features to which a Cox proportional hazards model could be fit with p<0.05) and SequentialFeatureSelector (sequentially adding single miRNA features to a multivariable Cox proportional hazards model based the best improvement in cross-validated concordance index, up to a specified maximum number of features) classes. Scikit-learn pipelines were used to couple the feature selection and training steps to create survival models and perform inference on new data. Random Survival Forest models from the scikit-survival package were used to train models using the parameter settings: n_estimators=1000, min_samples_split=10, min_samples_leaf=15, max_features=“sqrt”. Feature importance from Random Forest models was scored using the permutation based method from the package ELI5 (version 0.11.0) by estimating the reduction in model concordance index when removing the association between survival and each feature in turn (through random shuffling). Patients were stratified into high/low risk groups by the median risk score in the training data, based on the cutpoint definition of similar previously described prognostic gene expression signatures (Cho, J. Y. et al. (2011). Gene Expression Signature-Based Prognostic Risk Score in Gastric Cancer. Clin Cancer Res 17, 1850-1857; Hu, Z. et al. (2010). Serum MicroRNA Signatures Identified in a Genome-Wide Serum MicroRNA Expression Profiling Predict Survival of Non-Small-Cell Lung Cancer. J Clin Oncol 28, 1721-1726; Yu, S.-L. et al. (2008) MicroRNA Signature Predicts Survival and Relapse in Lung Cancer. Cancer Cell 13, 48-57). Prognostic performance of the miRisk score was assessed in a multivariable Cox proportional Hazards model with additional clinicopathological covariates (PD-L1, ECOG, Gender, Age, Therapy line, Substance, Histology, Smoking status, ANC, ALC). The survival function was estimated using the Kaplan-Meier method, and groups were compared with the log-rank test.

qRT-PCR

Catalogue and custom miRCury LNA miRNA PCR assays were ordered from Qiagen (Venlo, Netherlands) for biomarker miRNAs and housekeeping miRNAs (Table 4). 500 ng RNA was used for cDNA synthesis, or when the concentration was insufficient, up to 6.5 μl of RNA volume was used. cDNA was then diluted 1:80, 10 μl total volume reaction was set up according to manufacturer's instructions, and cycled according to the following protocol: 2 min at 95° C., 40×10 s at 95° C. 60 s at 56° C. in AB Flex 6 cycler in 384-well format. A melt curve analysis was performed at 60-95° C. The Cₜ values were determined and exported using QuantStudio RealTime PCR Software version 1.3. For the training cohort of 95 patients, the Cₜ values of the five miRisk miRNAs were determined in triplicates. ΔCₜ values were calculated by subtracting the mean of the Cₜ values of “housekeeping” miRNAs that were previously defined by NGS on the basis of low variance. Based on these ΔCₜ values the fold change (2^{ΔΔCt}) and t-test between samples from patients with low or high risk according to the miRisk score was calculated per signature miRNA.

TABLE 4
Housekeeping miRNAs selected for qPCR Normalization

| HK miRNA | mean RPM | variation_coef |
|---|---|---|
| hsa-miR-151a-5p | 1380.594439 | 0.316950798 |
| hsa-miR-181a-2-3p | 354.1860863 | 0.253508183 |
| hsa-miR-378a-3p | 418.6535476 | 0.371392777 |
| hsa-miR-625-3p | 695.3010592 | 0.337727072 |
| hsa-miR-6750-3p | 1.242074051 | 0.570468679 |
| hsa-miR-92a-3p | 108968.9911 | 0.286225534 |
| hsa-miR-941 | 1814.268118 | 0.287466556 |

### 2.2 Results

### Clinical Patient Characteristics

The Training Cohort consisted of 96 stage IV NSCLC patients who received anti-PD-1 immunomonotherapy (Table 5). 77.1% (n=74) received pembrolizumab and 22.9% (n=22) received nivolumab. The cohort comprised 58.3% adenocarcinomas (n=56), 28.1% squamous cell carcinomas (n=27), and 13.5% other NSCLCs (n=13), including NSCLC, NOS and large cell neuroendocrine lung carcinomas. 62.5% of the patients were male, 38.5% female. 91.7% of the patients were former or current smokers. 49% of the patients received first-line and 47.9% second-line therapy. Only 3.1% of the patients received immunotherapy in the third-line. 70.8% had PD-L1 TPS of 50% or higher, 20.8% between 1% and 49% and 8.3%<1%. 36.5% had an ECOG of 0, 58.3% of 1 and 5.2% of 2.

A cohort of 84 additional patients with similar characteristics were used as an independent Validation Cohort (see Table 5 below). Of this cohort, 52.4% of patients received pembrolizumab and 47.6% nivolumab. 25% received first-line therapy, 51.2% second-line, 15.5% third-line and 8.4% received fourth-line or higher. 59.5% were adenocarcinoma, 31.0% were squamous cell carcinoma and 9.5% were other NSCLCs. 48.8% were PD-L1 TPS≥50%, 32.1% were 1%-49% and 19% were ≤1%. 95.3% were current or former smokers. 39.3% were ECOG 0, 54.8% ECOG 1, 4.8% ECOG 2 and 1.2% ECOG 3.

TABLE 5
Cohort Overview

| | Training | Independent | |
|---|---|---|---|
| | Immunotherapy | Immunotherapy | Immunochemotherapy |
| Characteristic | (n = 96) | (n = B4) | (n = 139) |
| Sex. no. (%) | | | |
| Male | [symbol] 0 ( [symbol] %) | 51 ( [symbol] 0.7%) | [symbol] (66 [symbol] %) |
| Female | 36 ( [symbol] %) | 33 (39.3%) | [symbol] (33.1%) |
| Age at enrollment, yr | | | |
| Mean ± SD | [symbol] .4 | [symbol] 5 [symbol] 1 | [symbol] .0 |
| Median (range) | [symbol] ( [symbol] ) | [symbol] ( [symbol] ) | [symbol] (37 [symbol] ) |
| Histological Subtype, no. (%) | | | |
| Adenocarcinoma | 5 [symbol] (5 [symbol] .3%) | [symbol] ( [symbol] .5%) | 10 [symbol] ( [symbol] %) |
| Squamous cell carcinoma | 27 ( [symbol] %) | 26 (31 [symbol] %) | 18 (12.9%) |
| Other | 13 (13.5%) | 8 ( [symbol] 5%) | 15 (10. [symbol] %) |
| ECOG performance status, no, (%) | | | |
| 0 | 35 ( [symbol] %) | 33 ( [symbol] %) | [symbol] (40. [symbol] %) |
| 1 | 5 [symbol] ( [symbol] .3%) | 4 [symbol] ( [symbol] %) | 79 ( [symbol] %) |
| 2 | [symbol] ( [symbol] %) | 4 (4.8%) | 3 (2.2%) |
| 3 | [symbol] | 1 (1.2%) | 1 ( [symbol] 7%) |
| Smoking status, no. (%) | | | |
| Never | 8 (8. [symbol] %) | 4 (4.8%) | 12 ( [symbol] ) |
| Former | [symbol] ( [symbol] .3%) | [symbol] (54.8%) | [symbol] 4 ( [symbol] %) |
| Current | 32 ( [symbol] 2%) | [symbol] 4 (40. [symbol] %) | [symbol] ( [symbol] %) |
| Therapy, no. (%) | | | |
| Nivolumab | [symbol] ( [symbol] %) | 40 ( [symbol] %) | [symbol] |
| Pembrol [symbol] umab | 74 (77 [symbol] %) | 44 ( [symbol] %) | [symbol] |
| Platinum doublet [symbol] Pembrol [symbol] umab | [symbol] | [symbol] | 139 (100%) |
| Therapy Line, no. (%) | | | |
| 1 | 47 ( [symbol] 0%) | 21 ( [symbol] %) | 124 ( [symbol] 2%) |
| 2 | [symbol] (47.9%) | 43 ( [symbol] %) | 15 (1 [symbol] %) |
| 3 | 3 ( [symbol] %) | 13 ( [symbol] %) | [symbol] |
| >3 | [symbol] | 7 ( [symbol] %) | [symbol] |
| PD-L1 TPS, no. % | | | |
| [symbol] | [symbol] (70 [symbol] %) | 41 ( [symbol] %) | [symbol] 1 (22 [symbol] %) |
| 1-49 | 2 [symbol] ( [symbol] %) | 27 (32 [symbol] %) | [symbol] (27 [symbol] %) |
| >1 | [symbol] ( [symbol] 3%) | 1 [symbol] ( [symbol] %) | [symbol] ( [symbol] 4%) |

[symbol]

indicates data missing or illegible when filed

A third cohort of 139 patients receiving non-immunomonotherapy was used as an independent control group (Table 5). These patients received immune-chemo combination therapy of platinum doublet and pembrolizumab. This cohort comprised 76.3% adenocarcinomas, 12.9% squamous cell carcinomas, and 10.8% other NSCLCs. 89.2% were first-line patients, 10.8% second-line. 50.4% had PD-L1 TPS<1%, 27.3% between 1% and 49%, and 22.3%≥50%, 40.3% had an ECOG status of 0, 56.8% of 1, 2.2% of 2 and 0.7% of 3. 91.4% were former or current smokers.

A fourth independent cohort of 19 stage IV NSCLC patients was included and served as additional validation cohort.

Building a miRNA-Based Risk Model for Overall Survival (OS) Under Immunotherapy

miRNA expression from patients in the Training Cohort (96 patients) were used to develop a model to predict OS of stage IV NSCLC patients receiving single agent immunotherapy of either pembrolizumab or nivolumab. A computational pipeline based on that described by Shukla et al., was created that performed initial filtering of features based on Cox univariable association with survival, before a further stepwise multivariable Cox regression was used to create a multiple miRNA signature (Shukla, S. et al. (2016) Development of a RNA-Seq Based Prognostic Signature in Lung Adenocarcinoma. J Natl Cancer I 109, djw200).

More specifically, miRNA features to which a statistically significant (p<0.05) univariable Cox proportional hazards model could be fit were first selected, resulting in the selection of 44 miRNA shortlist (see Table 1 above in Example 1) from 600 total miRNAs. Next, forward sequential feature selection was performed to build multivariable Cox models incorporating increasing numbers of miRNA expression data. To choose a small subset of features that would be amenable to alternative low throughput quantification platforms (e.g. qRT-PCR) and avoid model overfitting, a cross-validated grid search (testing models with 3-10 features) was performed which identified a subset of five features that resulted in optimal model performance. Finally, a Cox proportional hazards model was fit to the entire training dataset using the selected five miRNA features to generate the following risk score: miRisk=(log(miR-2115-3p RPM+1)×1.870)+(log(miR-218-5p RPM+1)×0.907)+(log(miR-224-5p RPM+1)×0.495)+(log(miR-4676-3p RPM+1)×1.309)+(log(miR-6503-5p RPM+1)×1.159).

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

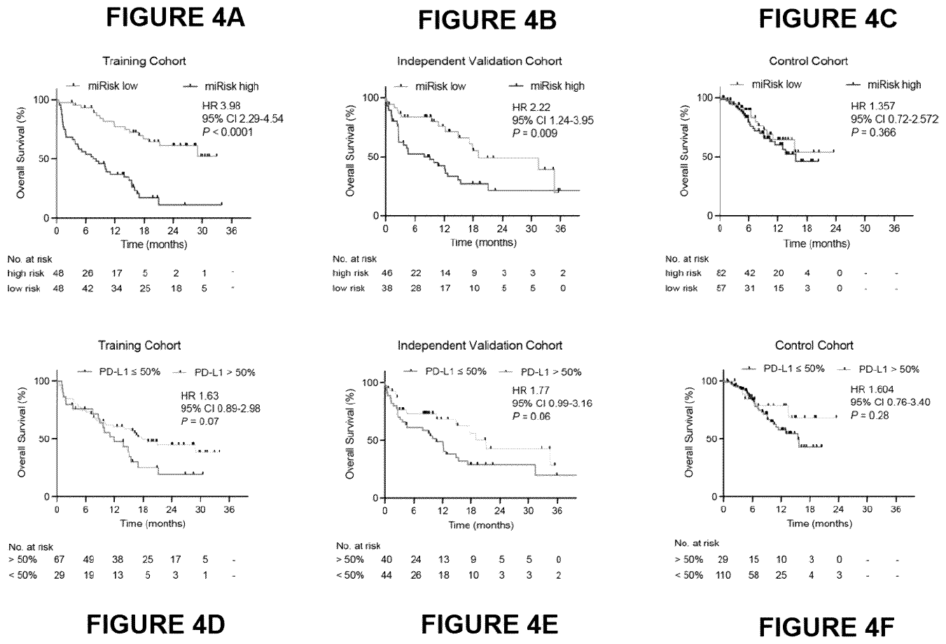

Risk scores were calculated for all 96 patients within the Training Cohort (see Table 5 above) and patients were separated into low/high risk groups based on the median risk score threshold. Specifically, a miRisk score threshold=−0.13906398900641143 was calculated to stratify patients as having a high or low risk (high risk=miRisk >−0.13906398900641143). Patients within the low-risk group survive for significantly longer than those in the high-risk group (HR 3.98, 95% CI 2.29-4.54; P=1.560×10⁻⁷) (FIG. 4a). To test the generalizable performance to new data, a miRisk score analysis of the 84 patients in the Validation Cohort (Table 5) was performed. It was shown that patients in the low-risk group survived for significantly longer than those in the high-risk group (HR 2.22, 95% CI 1.24-3.95; P=0.00902) (FIG. 4b).

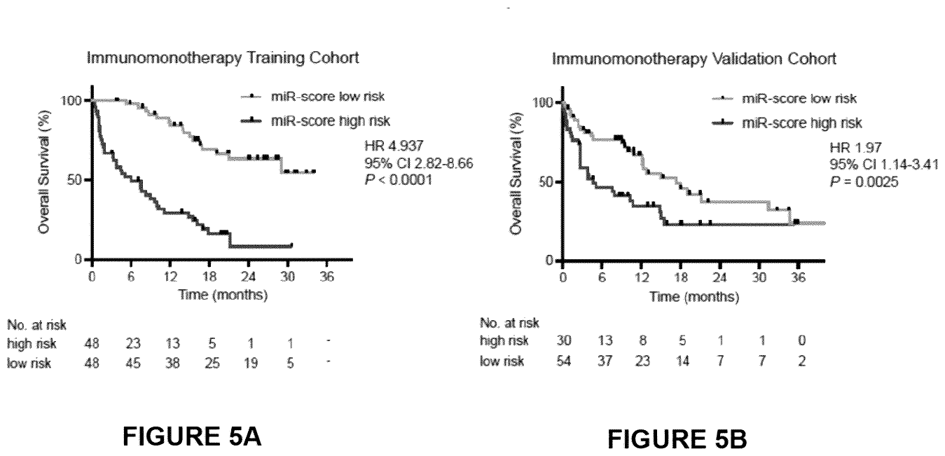

Non-linear models offer the promise to capture more complex relationships between covariates and the outcome of interest; in our case, miRNAs and the response to immunotherapy. To explore whether such models may lead to increased predictive performance, a Random Forest classifier was trained and evaluated on the dataset (Pölsterl, 2020, supra). A Random Forest model was fit to all 600 miRNA expression features within the training cohort, without any prior feature selection. A highly significant difference in the length of overall survival was observed between low/high risk groups within the training cohort (FIG. 5a). The Random Forest model displays generalised performance in the Validation Cohort in which it predicts a low-risk group of patients with significantly increased survival, relative to high-risk patients (HR 1.97, 95% CI 1.14-3.41; P=0.0025) (FIG. 5b). Finally, permutation-based feature importance was used to identify the most informative miRNA features used by the Random Forest classifier. ⅗ of the miRisk miRNAs were observed amongst the most important features used by the Random Forest model including miR-2115-3p, the feature from the miRisk model with the greatest weighting (Table 6).

TABLE 6
Overall survival of low and high-risk patients
stratified by Random Forest model.

| Feature | Weight |
|---|---|
| hsa-miR-2115-3p | 0.0089 ± 0.0145 |
| hsa-miR-150-5p | 0.0049 ± 0.0081 |
| hsa-miR-224-5p | 0.0048 ± 0.0046 |
| hsa-miR-218-5p | 0.0041 ± 0.0031 |
| hsa-miR-155-5p | 0.0041 ± 0.0032 |
| hsa-miR-342-3p | 0.0035 ± 0.0048 |
| hsa-miR-338-3p | 0.0033 ± 0.0025 |
| hsa-miR-23b-3p | 0.0025 ± 0.0047 |
| hsa-miR-1180-3p | 0.0025 ± 0.0022 |
| hsa-miR-143-3p | 0.0024 ± 0.0048 |
| hsa-miR-6505-5p | 0.0024 ± 0.0127 |
| hsa-miR-5001-3p | 0.0023 ± 0.0038 |
| hsa-miR-10399-5p | 0.0022 ± 0.0032 |
| hsa-miR-6885-3p | 0.0022 ± 0.0025 |
| hsa-miR-31-5p | 0.0022 ± 0.0012 |
| hsa-miR-1273h-3p | 0.0022 ± 0.0031 |
| hsa-miR-25-3p | 0.0022 ± 0.0042 |
| hsa-miR-4772-3p | 0.0021 ± 0.0082 |
| hsa-miR-16-1-3p | 0.0021 ± 0.0022 |
| hsa-miR-3667-3p | 0.0020 ± 0.0036 |

Random Forest feature importance as assessed by permutation testing. Weights indicate the decrease in concordance index attributed to each feature. Bold miRNAs are shared with the miRisk signature.

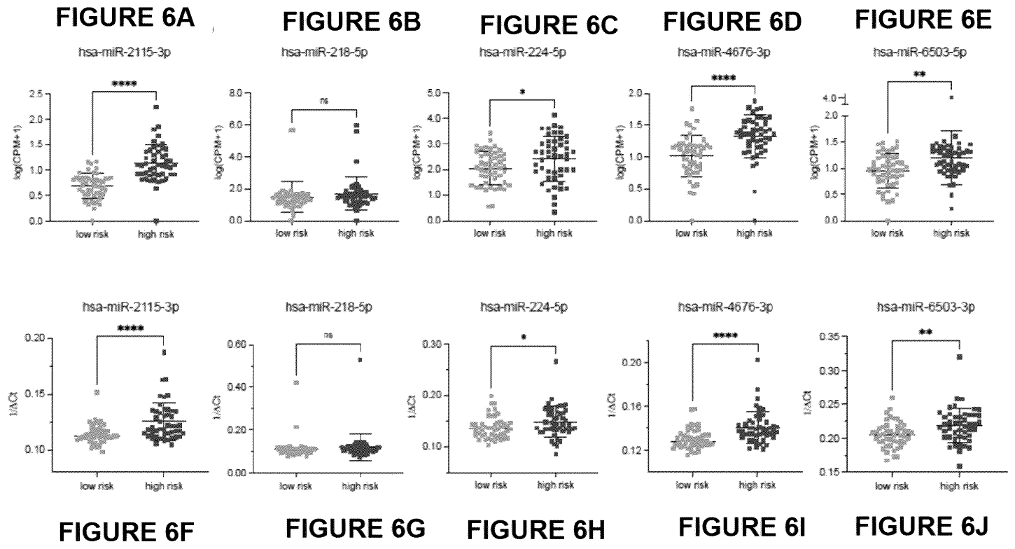

Four of the five miRNAs that contribute to the miRisk score are observed to be significantly upregulated in high-risk patients (FIG. 6a-e). Only miR-218-5p appears to be minimally upregulated in high-risk patients but this difference was not statistically significant (FIG. 6b). To technically validate our findings using an orthogonal method, the miRNA expression profiles of 95 patients from the training cohort were remeasured using qRT-PCR. The qPCR analysis recapitulated the NGS results for all five miRNAs with identical direction of change and similar degrees of significance (FIG. 6f-j).

Together, two entirely different models were trained on our dataset and convergence on a subset of informative miRNA features was observed. The quantification of features were technically validated by showing consistency between measurements from both NGS and qRT-PCR. Given the greater hazard ratio between risk groups predicted by the miRisk Cox based model and its ease of interpretability it was chosen for further investigation.

miRisk Score is Predictive not Prognostic

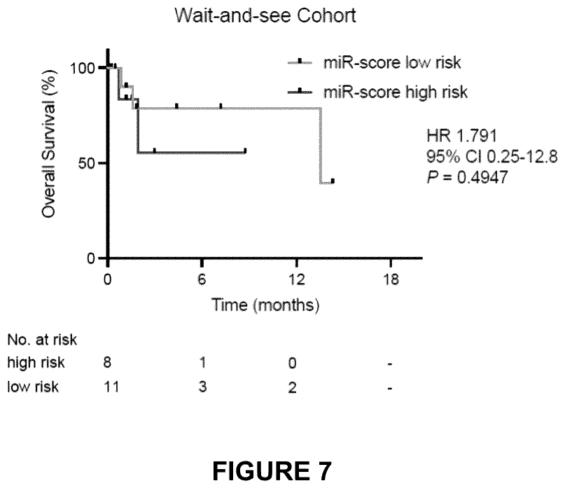

In order to discern a potential predictive vs. prognostic importance of the miRisk score, the model performance in two further distinct control patient cohorts consisting of patients receiving non-ICI monotherapy was explored. 139 patients from the Immunochemotherapy Validation Cohort (Table 5) were stratified and no significant difference in survival between those in the low risk versus high-risk groups was observed (HR 1.357, 95% CI 0.72-2.572; P=0.366) (FIG. 4c). This is consistent with previous findings that immunotherapy biomarkers (tumor PD-L1) do not predict the survival advantages from adding immunotherapy to chemotherapy alone (Gandhi, L. et al. (2018). Pembrolizumab plus Chemotherapy in Metastatic Non-Small-Cell Lung Cancer. New Engl J Med 378, 2078-2092). The model was tested in a further independent control cohort consisting of 19 stage IV NSCLC patients recruited in Grosshansdorf, Germany and who opted to undergo watchful waiting without treatment. Following miRisk score calculation, no significant difference in OS was observed between risk groups (FIG. 7). Again, this provides evidence against a prognostic model and in favor of a specific prediction of immunotherapy response.

Evaluating the miRisk Score in a Clinical Context

To explore the association between the miRisk score, and other clinical covariates with respect to overall survival, both univariable and multivariable Cox regression was performed within the immunomonotherapy Validation Cohort (Table 7). In addition to the miRisk score, clinicopathological covariates of relevance to immunotherapy response was included in the Cox models. In univariable analysis, significant associations between only ECOG (HR 2.90, 95% CI 1.47-5.73; P=0.00) and the miRisk score (HR 2.23, 95% CI 1.2-4.15; P=0.01) was observed. A non-significant association between tumour PD-L1<50% and survival in the immunotherapy Validation Cohort was observed, with the expected trend towards increased risk with lower PD-L1 (HR 1.78, 95% CI 0.97-3.28; P=0.06) (Table 7). A non-significant protective effect was observed from both low ANC, and high ALC at univariable analysis (Table 7), which is consistent with a published predictive signature derived from peripheral blood counts (Tanizaki, J. et al. (2018). Peripheral Blood Biomarkers Associated with Clinical Outcome in Non-Small Cell Lung Cancer Patients Treated with Nivolumab. Journal of Thoracic Oncology: Official Publication of the International Association for the Study of Lung Cancer 13, 97-105). This effect is diminished when controlling for other covariates in the multivariable analysis. Both ECOG and the miRisk score remain as the only two significant independent predictors of overall survival in multivariable Cox regression (Table 7).

TABLE 7
Univariable and multivariable Cox regression analysis

| Overall survival | Univariable Analysis | | | Multivariable Analysis | | |
|---|---|---|---|---|---|---|
| Covariate | HR | 95% CI | P-value | HR | 95% CI | P-value |
| PD-L1 (<50%) | 1.76 | 0.97-3.26 | 0.06 | 2.07 | 0.62-6.86 | 0.23 |
| ECOD (>0) | 2.90 | 1.47-5.73 | 0.00 | 5.27 | 2.43-11.41 | 0.00 |
| Gender (male vs female) | 1.04 | 0.57-1.9 | 0.89 | 0.74 | 0.38-1.45 | 0.39 |
| Age (>75 years) | 1.36 | 0.63-2.93 | 0.43 | 1.36 | 0.59-3.23 | 0.45 |
| Therapy line incl. [symbol] V (>2) | 1.35 | 0.72-2.54 | 0.35 | 1.31 | 0.66-2.59 | 0.43 |
| Substance (Pembro vs Nivo) | 0.67 | 0.37-1.22 | 0.19 | 1.31 | 0.4-4.24 | 0.66 |
| Histology (non-adeno vs adeno) | 1.52 | 0.85-2.72 | 0.16 | 1.46 | 0.71-3.11 | 0.3 [symbol] |
| Smoking status (ever smoker vs other) | 1.54 | 0.37-6.39 | 0.55 | 5.14 | 1.03-25.56 | 0.05 |
| ANC (>7. [symbol] ) | 1.67 | 0.82-3.01 | 0.18 | 0.86 | 0.41-1.6 | 0.69 |
| Ly (>1) | 0.55 | 0.31-1 | 0.05 | 0. [symbol] 3 | 0.34-1.17 | 0.14 |
| mIR-score (high vs low) | 2.23 | 1.2-4.15 | 0.01 | 2.46 | 1.06-5.71 | 0.04 |

[symbol]

indicates data missing or illegible when filed

miRisk Score Outperforms PD-L1 Histology as a Predictive Biomarker

Tumor PD-L1 expression is currently the only approved companion diagnostic biomarker for the prediction of immunotherapy response in NSCLC and recognised in the current ASCO and ESMO guidelines when selecting treatment options. Patients within both the training and validation cohorts were stratified based on PD-L1 tumor staining levels (>50%) and their survival was assessed by Kaplan-Meier analysis (FIG. 4d-f). There is a trend towards longer overall survival in the high PD-L1 patients, but this does not reach significance in any cohort as opposed to the miRisk score which was observed to be a significant predictor. This would be consistent with a recent meta-analysis that found PD-L1 to be predictive of immunotherapy response in only approximately 30% of patients across a range of tumour types (Davis, A. A. and Patel, V. G. (2019). The role of PD-L1 expression as a predictive biomarker: an analysis of all US Food and Drug Administration (FDA) approvals of immune checkpoint inhibitors. J Immunother Cancer 7, 278.).

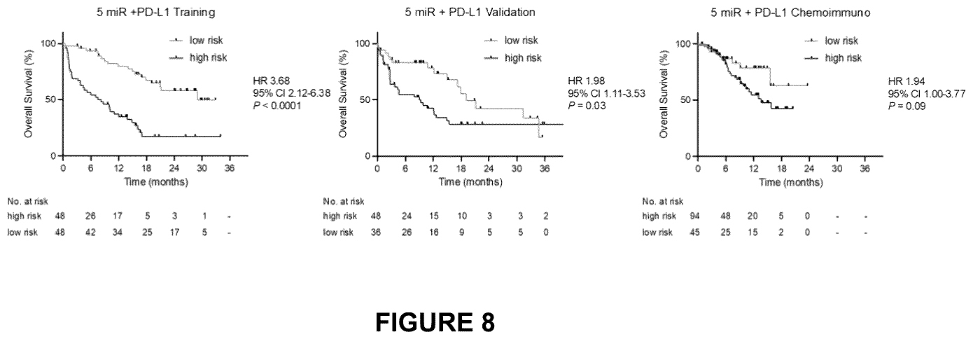

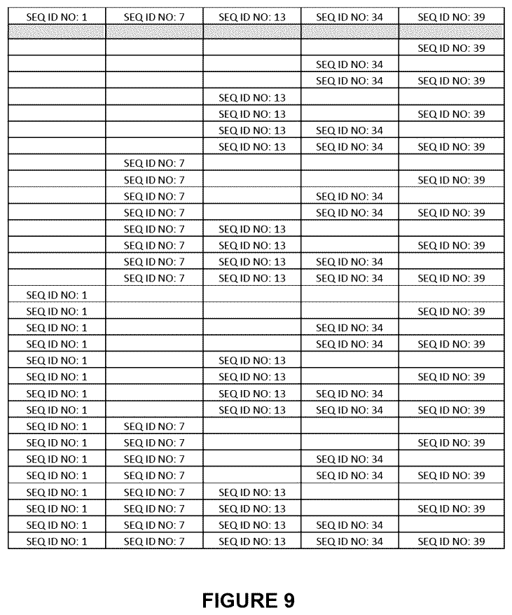

The miR-score model was retrained using tumor PD-L1 in addition to the five previously identified signature miRNAs. No significant improvement in predictive performance from the addition of tumour PD-L1 expression levels was observed (FIG. 8). This clearly shows that the approach presented herein allows a quick and accurate clinical response prediction and survival prognosis in cancer diseases (independent from PD-L1 expression).

### 3. Example 3

### 3.1 Methods

This study included a total of 155 prospectively recruited patients with newly diagnosed stage IV non-small-cell lung carcinoma (NSCLC) and PD-L1 Tumor Proportion Score (TPS)≥50%, whose blood samples were provided by the Lungenbiobank Heidelberg and Biobank Nord according to the pertinent regulations and with the approval of local ethics committees at Heidelberg University (S-296/2016, S-089/2019, S-916/2019) and Grosshansdorf Hospital (AZ 12-238, AZ 19-286). Blood sample processing and generation of small RNA expression profiles via small RNA sequencing are described in detail above. All anonymized small RNA sequencing data has been deposited at the European Nucleotide Archive under accession number PRJEB50502. Survival analyses were performed in Python (3.8.8), using the packages scikit-survival (version 0.15.1) and Lifelines (version 0.26.0). Kaplan-Meier analysis was performed in GraphpadPrism (version 9.3.1).

### 3.2 Results

This study included 124 stage IV NSCLC immunotherapy- (IO-) treated patients, divided into training (n=68) and validation (n=56) cohorts according to the time-point of sample collection, and a control cohort treated with immunochemotherapy (ICT) (n=31) (Table 8). These three cohorts display broadly similar clinicopathological characteristics.

TABLE 8
Cohort overview

| | Training | | Independent Validation | | Control | |
|---|---|---|---|---|---|---|
| Treatment | Immunotherapy | | Immunotherapy | | Immunochemotherapy | |
| Characteristic | (n = 68) | | (n = 56) | | (n = 31) | |
| Site | | | | | | |
| Heidelberg | 68 | | 41 | | 31 | |
| Großhansdorf | — | | 15 | | — | |
| Sex, no. (%) | | | | | | |
| Male | 40 | (58.8%) | 38 | (67.9%) | 20 | (64.5%) |
| Female | 28 | (41.2%) | 18 | (32.1%) | 11 | (35.5%) |
| Age at enrollment, yr | | | | | | |
| Mean ± SD | 68.0 ± 10.0 | | 68.2 ± 8.8 | | 62.5 ± 10.6 | |
| Median (range) | 67.7 | (38.9-86.7) | 69.1 | (51.2-87.0) | 64.7 | (37.6-78.6) |
| Histological Subtype, no. (%) | | | | | | |
| Adenocarcinoma | 43 | (63.2%) | 34 | (60.7%) | 25 | (80.6%) |
| Squamous cell carcinoma | 18 | (26.5%) | 18 | (32.1%) | 3 | (9.7%) |
| Other | 7 | (10.3%) | 4 | (7.1%) | 3 | (9.7%) |
| ECOG performance status, no. (%) | | | | | | |
| 0 | 23 | (33.8%) | 22 | (39.3%) | 10 | (32.3%) |
| 1 | 42 | (61.8%) | 28 | (50.0%) | 20 | (64.5%) |
| 2 | 3 | (4.4%) | 3 | (5.4%) | 1 | (3.2%) |
| 3 | — | | 3 | (5.4%) | 0 | |
| NA | | | 0 | | | |
| Smoking status, no. (%) | | | | | | |
| Never | 6 | (8.8%) | 1 | (1.8%) | 3 | (9.7%) |
| Former | 36 | (52.9%) | 37 | (66.1%) | 14 | (45.2%) |
| Current | 26 | (38.2%) | 18 | (32.1%) | 14 | (45.2%) |
| Therapy, no. (%) | | | | | | |
| Nivolumab | 6 | (8.8%) | 4 | (7.1%) | — | |
| Pembrolizumab | 62 | (91.2%) | 52 | (92.9%) | — | |
| Platinum doublet + Pembrolizumab | — | | — | | 31 | (100%) |
| Therapy Line, no. (%) | | | | | | |
| 1 | 46 | (67.6%) | 35 | (62.5%) | 27 | (87.1%) |
| 2 | 21 | (30.9%) | 15 | (26.8%) | 4 | (12.9%) |
| 3 | 1 | (1.5%) | 3 | (5.4%) | — | |
| >3 | — | | 3 | (5.6%) | — | |
| PD-L1 TPS, no. % | | | | | | |
| >50 | 68 | (100%) | 56 | (100%) | 31 | (100%) |

| | | | | | |
|---|---|---|---|---|---|
| 1-50 | 0 | (0%) | 0 | (0.0%) | 0 |
| <1 | 0 | (0%) | 0 | (0.0%) | 0 |

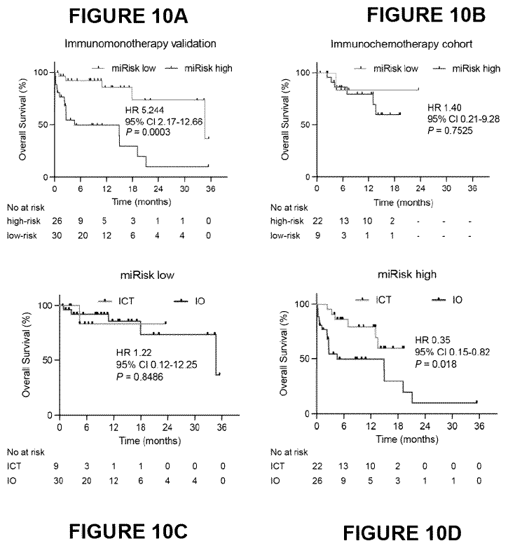

A significantly longer overall survival (OS) in the miRisk low group of patients in the training cohort (HR 3.65, 95% CI 1.84-7.24; P<0.001) was observed (data not shown), which was confirmed in the validation cohort (HR 5.24, 95% CI 2.17-12.66; P<0.001) (FIG. 10a). In contrast, the miRisk score was not associated with overall survival (OS) in patients in the immunochemotherapy (ICT) control cohort (HR 1.40, 95% CI 0.21-9.28; P=0.753) (FIG. 10b), consistent with previous reports that 10-specific biomarkers do not predict response to ICT.

In order to explore the utility of the miRisk score as a complementary diagnostic for immunotherapy (10) versus immunochemotherapy (ICT) treatment decisions, the validation and control cohorts was merged to explore the interaction between overall survival (OS) and treatment.

In the miRisk low patients, there was no OS difference between those treated with IO or ICT (HR 1.22, 95% CI 0.12-12.25; P=0.849) (FIG. 10c). In contrast, in miRisk high patients, a significantly improved OS in patients receiving ICT compared to IO was observed (HR 0.35, 95% CI 0.15-0.82; P=0.018) (FIG. 10d). The interaction between treatment and miRisk score in a multivariable Cox Proportional Hazards model was significant (P=0.036), suggesting the miRisk score is both prognostic and predictive for the efficacy of IO (data not shown).

Finally, multivariable Cox Proportional Hazards models were used to investigate the performance of the miRisk score when controlling for other relevant clinicopathological covariates. This demonstrated that the miRisk score showed a stronger association with OS of IO-treated patients (HR 4.45, 95% CI 1.58-12.49; P=0.004) than PD-L1 TPS, tumour histology and ECOG performance status (Table 9).

TABLE 9
Univariable and multivariable Cox regression analysis of miRisk and clinical covariates

| Overall survival | Univariable Analysis | | | Multivariable Analysis | | |
|---|---|---|---|---|---|---|
| Covariate | HR | 95% CI | p-value | HR | 95% CI | p-value |
| Training Cohort | | | | | | |
| ECOG | 1.39 | 0.76-2.52 | 0.281 | 0.84 | 0.45-1.58 | 0.597 |
| Histology (non-adeno vs adeno) | 1.09 | 0.55-2.17 | 0.806 | 1.39 | 0.69-2.82 | 0.360 |
| PD-L1 TPS | 0.99 | 0.97-1.01 | 0.433 | 0.96 | 0.93-0.99 | 0.004 |
| miRisk (high vs low) | 3.84 | 1.86-7.95 | <0.001 | 7.86 | 3.13-19.73 | <0.001 |
| Independent validation cohort | | | | | | |
| ECOG | 3.53 | 1.55-8.05 | 0.003 | 3.30 | 1.24-8.79 | 0.017 |
| Histology (non-adeno vs adeno) | 1.89 | 0.79-4.5 | 0.151 | 1.76 | 0.68-4.57 | 0.246 |
| PD-L1 TPS | 1.00 | 0.97-1.03 | 0.753 | 1.01 | 0.98-1.04 | 0.650 |
| miRisk (high vs low) | 5.37 | 1.96-14.74 | 0.001 | 4.45 | 1.58-12.49 | 0.005 |
| Control Cohort | | | | | | |
| ECOG | 3.70 | 0.85-16.06 | 0.081 | 3.94 | 0.9-17.19 | 0.068 |
| Histology (non-adeno vs adeno) | 1.10 | 0.12-9.88 | 0.935 | 1.69 | 0.17-17.05 | 0.658 |
| PD-L1 TPS | 1.00 | 0.94-1.05 | 0.906 | 1.01 | 0.95-1.07 | 0.765 |
| miRisk (high vs low) | 1.41 | 0.17-11.91 | 0.754 | 1.41 | 0.16-12.39 | 0.755 |

In particular, fitting the multivariable Cox proportional hazards model to the training cohort using the 5 previously identified miRNAs as input features led to a revised risk score with adjusted weights; miRisk=(ln(miR-2115-3p RPM+1)×2.190467)+(ln (miR-218-5p RPM+1)×0.303095)+(ln (miR-224-5p RPM+1)×0.598415)+(ln (miR-4676-3p RPM+1)×1.101122)+(ln (miR-6503-5p RPM+1)×0.958823).

The nucleotide sequences of miR-2115-3p, miR-218-5p, miR-224-5p, miR-4676-3p, and miR-6503-5p are shown in the appended sequence listing as SEQ ID NOs: 1, 39, 13, 7, and 34, respectively.

Specifically, a miRisk cut-off score/threshold of −0.07250442496171916 was calculated which allows to stratify patients as patients who will (likely) benefit from immunochemotherapy (miRisk score >−0.07250442496171916, rounded off −0.073) or to stratify patients as patients who will (likely) benefit from immunochemotherapy or immunotherapy (miRisk score ≤−0.07250442496171916, rounded off −0.073).

In summary, the above results show that the miRisk score cut-off/threshold allows to distinguish a group of PD-L1 high, stage IV NSCLC patients to benefit from adding chemotherapy to immunotherapy. In other words, the miRisk cut-off score/threshold allows to identify a group of high-risk patients who will (likely) benefit from treatment with ICT as opposed to IO. Thus, the miRisk cut-off score/threshold supports treatment decisions as a complementary diagnostic.

1. A method of predicting a response to lung cancer immunotherapy of a patient suffering from lung cancer, comprising the step of:
determining the level of at least one miRNA in a blood sample of a patient suffering from lung cancer, wherein the at least one miRNA has a nucleotide sequence selected from the group consisting of SEQ ID NO: 1 to SEQ ID NO: 44, a fragment thereof, and a sequence having at least 80% sequence identity thereto.

2. The method of claim 1, wherein the at least one miRNA has a nucleotide sequence selected from
(i) the group consisting of SEQ ID NO: 1 to SEQ ID NO: 8, a fragment thereof, and a sequence having at least 80% sequence identity thereto, and/or
(ii) the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto.

3. (canceled)

4. The method of claim 1, wherein the level of the at least one miRNA is compared to a reference level of said at least one miRNA, wherein the reference level is the level of the at least one miRNA determined by measuring a number of reference blood samples from subjects suffering from lung cancer who are non-responders to said lung cancer immunotherapy, and wherein
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80% sequence identity thereto below the reference level indicates that the patient will respond to said lung cancer immunotherapy, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80% sequence identity thereto above the reference level indicates that the patient will respond to said lung cancer immunotherapy.

5-8. (canceled)

9. The method of claim 1, wherein the level of the at least one miRNA is compared to an empirically determined cut-off score, which allows to classify the patient as patient who will respond to lung cancer immunotherapy or as a patient who will not respond to lung cancer immunotherapy, and wherein the cut-off score is determined by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto.

10-13. (canceled)

14. The method of claim 1, wherein the lung cancer immunotherapy is accompanied with the administration of at least one immune checkpoint inhibitor.

15. The method of claim 14, wherein the at least one immune checkpoint inhibitor is selected from the group consisting of an inhibitor targeting PD-1, PD-L1, PD-L2, CTLA-4, LAG3, TIGIT, CD73 and intrinsic checkpoint blockades.

16-21. (canceled)

22. The method of claim 1, wherein the lung cancer is non-small-cell lung carcinoma (NSCLC).

23. A method of providing a survival prognosis to a patient suffering from lung cancer who is eligible to immunotherapy comprising the step of:
determining the level of at least one miRNA in a blood sample of a patient suffering from lung cancer, wherein the at least one miRNA has a nucleotide sequence selected from the group consisting of SEQ ID NO: 1 to SEQ ID NO: 44, a fragment thereof, and a sequence having at least 80% sequence identity thereto.

24. The method of claim 23, wherein the at least one miRNA has a nucleotide sequence selected from
(i) the group consisting of SEQ ID NO: 1 to SEQ ID NO: 8, a fragment thereof, and a sequence having at least 80% sequence identity thereto, and/or
(ii) the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto.

25. (canceled)

26. The method of claim 23, wherein the level of the at least one miRNA is compared to a reference level of said at least one miRNA, wherein the reference level is the level of the at least one miRNA determined by measuring a number of reference blood samples from subjects suffering from lung cancer having a known poor survival prognosis, and wherein
the level of the at least one miRNA having a nucleotide sequence selected from the group consisting of SEQ ID NO: 1 to SEQ ID NO: 23, SEQ ID NO: 25, SEQ ID NO: 26, SEQ ID NO: 28 to SEQ ID NO: 35, SEQ ID NO: 37, SEQ ID NO: 39 to SEQ ID NO: 42, SEQ ID NO: 44, a fragment thereof, and a sequence having at least 80% sequence identity thereto below the reference level indicates that the patient has a good survival prognosis, or
the level of the at least one miRNA having a nucleotide sequence selected from the group consisting of SEQ ID NO: 24, SEQ ID NO: 27, SEQ ID NO: 36, SEQ ID NO 38, SEQ ID NO: 43, a fragment thereof, and a sequence having at least 80% sequence identity thereto above the reference level indicates that the patient has a good survival prognosis.

27-30. (canceled)

31. The method of claim 23, wherein the level of the at least one miRNA is compared to an empirically determined cut-off score, which allows to classify the patient as having a good or poor survival prognosis, and wherein the cut-off score is determined by the weighted sum of one or more miRNAs selected from the group consisting of SEQ ID NO: 1, SEQ ID NO: 7, SEQ ID NO: 13, SEQ ID NO: 34, SEQ ID NO: 39, a fragment thereof, and a sequence having at least 80% sequence identity thereto.

32-33. (canceled)

34. The method of claim 31, wherein
the good survival prognosis is associated with a high chance/probability of surviving a certain time period, or
the poor survival prognosis is associated with a low chance/probability of surviving a certain time period.

35-37. (canceled)

38. The method of claim 23, wherein the patient is a patient to whom a lung cancer immunotherapy will be administered.

39-40. (canceled)

41. The method of claim 38, wherein the immunotherapy is accompanied with the administration of at least one immune checkpoint inhibitor.

42. The method of claim 41, wherein the at least one immune checkpoint inhibitor is selected from the group consisting of an inhibitor targeting PD-1, PD-L1, PD-L2, CTLA-4, LAG3, TIGIT, CD73 and intrinsic checkpoint blockades.

43-48. (canceled)

49. The method of claim 23, wherein the lung cancer is non-small-cell lung carcinoma (NSCLC).

50-81. (canceled)

82. A method of determining whether to treat a patient suffering from lung cancer with immunotherapy comprising the steps of:
(i) carrying out the method of claim 1, thereby identifying the patient as patient who will respond to lung cancer immunotherapy, and
(ii) assigning the patient to the lung cancer immunotherapy.

83. The method of claim 82, wherein the lung cancer is non-small-cell lung carcinoma (NSCLC).

84-102. (canceled)

103. A method of selecting a patient suffering from lung cancer to benefit from immunochemotherapy or immunotherapy comprising the step of:
determining the level of at least one miRNA in a blood sample of a patient suffering from lung cancer,
wherein the at least one miRNA has a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto, and
wherein the patient has optionally a PD-L1 Tumor Proportion Score (TPS) of ≥50%.

104-106. (canceled)

107. The method of claim 103, wherein the lung cancer is non-small-cell lung carcinoma (NSCLC).

108-109. (canceled)

110. The method of claim 103, wherein the level of the at least one miRNA is compared to a (miRisk) cut-off score, wherein
the level of the at least one miRNA above the (miRisk) cut-off score indicates that the patient will benefit from immunochemotherapy, or
the level of the at least one miRNA below or equal to the (miRisk) cut-off score indicates that the patient will benefit from immunochemotherapy or immunotherapy.

111. (canceled)

112. The method of claim 103, wherein the method further comprises the step of determining a (miRisk) score by summarizing the weighted levels of at least two, at least three, at least four, or five miRNAs having a nucleotide sequence selected from the group consisting of SEQ ID NO: 1 (miR-2115-3p), SEQ ID NO: 39 (miR-218-5p), SEQ ID NO: 13 (miR-224-5p), SEQ ID NO: 7 (miR-4676-3p), SEQ ID NO: 34 (miR-6503-5p), a fragment thereof, and a sequence having at least 80% sequence identity thereto.

113-118. (canceled)

119. A method of treating a patient suffering from lung cancer with immunochemotherapy or immunotherapy comprising the steps of:
(i) carrying out the method of claim 103, thereby identifying the patient as patient who will benefit from immunochemotherapy or immunotherapy, and
(ii) treating the patient with said therapy.

120-156. (canceled)


SEQUENCE LISTING (44 sequences)

SEQ ID NO: 1 - RNA, 22 nt, Homo sapiens
caucagaauucauggaggcuag

SEQ ID NO: 2 - RNA, 22 nt, Homo sapiens
cauugcacuugucucggucuga

SEQ ID NO: 3 - RNA, 22 nt, Homo sapiens
uccagcaucagugauuuuguug

SEQ ID NO: 4 - RNA, 23 nt, Homo sapiens
cccaguguuuagacuaucuguuc

SEQ ID NO: 5 - RNA, 22 nt, Homo sapiens
acuuguaugcuagcucagguag

SEQ ID NO: 6 - RNA, 22 nt, Homo sapiens
gcugcgcuuggauuucgucccc

SEQ ID NO: 7 - RNA, 22 nt, Homo sapiens
cacuguuucaccacuggcucuu

SEQ ID NO: 8 - RNA, 22 nt, Homo sapiens
ggauuccuggaaauacuguucu

SEQ ID NO: 9 - RNA, 22 nt, Homo sapiens
auaaagcuagauaaccgaaagu

SEQ ID NO: 10 - RNA, 21 nt, Homo sapiens
aucacauugccagggauuucc

SEQ ID NO: 11 - RNA, 22 nt, Homo sapiens
uguaacagcaacuccaugugga

SEQ ID NO: 12 - RNA, 22 nt, Homo sapiens
uaacugguugaacaacugaacc

SEQ ID NO: 13 - RNA, 25 nt, Homo sapiens
ucaagucacuagugguuccguuuag

SEQ ID NO: 14 - RNA, 23 nt, Homo sapiens
aucacauugccagggauuaccac

SEQ ID NO: 15 - RNA, 22 nt, Homo sapiens
augcugacauauuuacuagagg

SEQ ID NO: 16 - RNA, 22 nt, Homo sapiens
ugucaguuugucaaauacccca

SEQ ID NO: 17 - RNA, 20 nt, Homo sapiens
ggauccgagucacggcacca

SEQ ID NO: 18 - RNA, 22 nt, Homo sapiens
uauggaaagacuuugccacucu

SEQ ID NO: 19 - RNA, 21 nt, Homo sapiens
aauuacagauugucucagaga

SEQ ID NO: 20 - RNA, 21 nt, Homo sapiens
ucacagugaaccggucucuuu

SEQ ID NO: 21 - RNA, 21 nt, Homo sapiens
uucacaguggcuaaguuccgc

SEQ ID NO: 22 - RNA, 21 nt, Homo sapiens
uucacaguggcuaaguucugc

SEQ ID NO: 23 - RNA, 23 nt, Homo sapiens
guccaguuuucccaggaaucccu

SEQ ID NO: 24 - RNA, 22 nt, Homo sapiens
ucucccaacccuuguaccagug

SEQ ID NO: 25 - RNA, 21 nt, Homo sapiens
ucggcuguguaucucuguguc

SEQ ID NO: 26 - RNA, 22 nt, Homo sapiens
ucucugggccugugucuuaggc

SEQ ID NO: 27 - RNA, 21 nt, Homo sapiens
ucucuggucuuuccuugacag

SEQ ID NO: 28 - RNA, 22 nt, Homo sapiens
ccugcaacuuugccugaucaga

SEQ ID NO: 29 - RNA, 23 nt, Homo sapiens
ucuuugguuaucuagcuguauga

SEQ ID NO: 30 - RNA, 22 nt, Homo sapiens
ugagaccucuggguucugagcu

SEQ ID NO: 31 - RNA, 22 nt, Homo sapiens
agcuuccaugacuccugaugga

SEQ ID NO: 32 - RNA, 22 nt, Homo sapiens
cuuucagucggauguuuacagc

SEQ ID NO: 33 - RNA, 21 nt, Homo sapiens
aguggcaaagucuuuccauau

SEQ ID NO: 34 - RNA, 23 nt, Homo sapiens
aggucugcauucaaauccccaga

SEQ ID NO: 35 - RNA, 22 nt, Homo sapiens
uuagccaauuguccaucuuuag

SEQ ID NO: 36 - RNA, 23 nt, Homo sapiens
aucagggcuuguggaaugggaag

SEQ ID NO: 37 - RNA, 22 nt, Homo sapiens
uuggaauaggggauaucucagc

SEQ ID NO: 38 - RNA, 22 nt, Homo sapiens
cugguacaggccugggggacag

SEQ ID NO: 39 - RNA, 21 nt, Homo sapiens
uugugcuugaucuaaccaugu

SEQ ID NO: 40 - RNA, 22 nt, Homo sapiens
cuuggcaccuagcaagcacuca

SEQ ID NO: 41 - RNA, 22 nt, Homo sapiens
uuuugcgauguguuccuaauau

SEQ ID NO: 42 - RNA, 21 nt, Homo sapiens
gcgacccacucuugguuucca

SEQ ID NO: 43 - RNA, 22 nt, Homo sapiens
cuccccucucuuuccuguucag

SEQ ID NO: 44 - RNA, 21 nt, Homo sapiens
gcagcccagcugaggccucug